(19) United States
(12) Patent Application Publication
(10) Pub. No.: US 2026/0265834 A1
(43) Pub. Date: Sep. 10, 2026

(54) METHODS FOR PREDICTING EFFECTS OF DOSE INTENSE CHEMOTHERAPY TREATMENTS

(71) Applicants: BOARD OF REGENTS, THE UNIVERSITY OF TEXAS SYSTEM, Austin, TX (US); CORNELL UNIVERSITY, Ithaca, NY (US); DANA-FARBER CANCER INSTITUTE, INC., Boston, MA (US)

(72) Inventors: William Fraser SYMMANS, Houston, TX (US); Otto METZGER, Boston, MA (US); Karla BALLMAN, St. Paul, MN (US); Lili DU, Houston, TX (US); Chunxiao FU, Pearland, TX (US)

(73) Assignees: BOARD OF REGENTS, THE UNIVERSITY OF TEXAS SYSTEM, Austin, TX (US); CORNELL UNIVERSITY, Ithaca, NY (US); DANA-FARBER CANCER INSTITUTE, INC., Boston, MA (US)

(21) Appl. No.: 19/473,231

(22) PCT Filed: Apr. 29, 2024
(86) PCT No.: PCT/US2024/026859

§ 371 Date: Oct. 7, 2025

Related U.S. Application Data
(60) Provisional application No. 63/499,035, filed on Apr. 28, 2023.

(51) Int. Cl. C12Q 1/6886; A61K 31/337; A61K 31/675; A61K 31/704; A61P 35/00; C07K 16/28

(52) U.S. Cl. CPC C12Q 1/6886; A61K 31/337; A61K 31/675; A61K 31/704; A61P 35/00; C07K 16/2818; C07K 16/2827; C12Q 2600/106; C12Q 2600/112; C12Q 2600/158

(57) ABSTRACT

Provided herein are methods of determining sensitivity to dose intense chemotherapy regimens based upon an index of estrogen receptor (ER)- and progesterone receptor (PR)-related genes, referred to as the sensitivity to endocrine therapy index (SET_{ER/PR} index). Further provided are methods of treating cancer patients determined to be sensitive to dose intense chemotherapy therapy by the SET_{ER/PR} index. Also provided herein are methods for determining prognosis based on the SET_{2,3} index.

This application claims benefit of priority to U.S. Provisional Application Ser. No. 63/499,035 filed Apr. 28, 2023, the entire contents of which are hereby incorporated by reference.

### SEQUENCE LISTING

This application contains a Sequence Listing XML, which has been submitted electronically and is hereby incorporated by reference in its entirety. Said XML Sequence Listing, created on Apr. 29, 2024, is named UTFCP1519WO.xml and is 520,192 bytes in size.

### BACKGROUND

### 1. Field

The present invention relates generally to the fields of molecular biology and medicine. More particularly, it concerns methods, biomarkers, kits, and the like used in determining the effects of dose intense chemotherapy treatments.

### 2. Description of Related Art

In the following discussion certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

Contemporary adjuvant chemotherapy regimens for breast cancer combine treatments that are believed to add to survival outcomes. In the 1980s, trials indicated that concurrent addition of an anthracycline, either doxorubicin (adriamycin) or epirubicin, to cyclophosphamide+/−fluorouracil improved survival outcomes (e.g. AC, FAC, EC, FEC). Then, in the 1990s, trials suggested that sequential or concurrent addition of a taxane, either paclitaxel or docetaxel, improved survival from 3-weekly anthracycline-based regimens (e.g. AC/T or T/AC, or TAC). Thereafter, in the 2000's, trials suggested that further improvement was achieved by intensifying the dosing schedule for paclitaxel to a “dose-intense” higher dose, 2-weekly regimen requiring hematological colony stimulating factor to support the bone marrow (e.g. ddAC/T) or a lower-dose weekly regimen (e.g. wT/FAC, AC/wT). Little, however, is understood about defined populations that experience a benefit, or that do not experience a benefit from such dose intense chemotherapy regimens. The present disclosure provides methods for identifying patients that benefit from such treatment schedules.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The CALGB 9741 trial led by the National Cancer Institute Cancer and Leukemia Group B demonstrated improved disease-free survival (DFS) and overall survival (OS) from dose-intense chemotherapy administered every 2 weeks (ddAC/T) compared to conventional chemotherapy dosing every 3 weeks (AC/T) for patients with node-positive breast cancer. However, survival was not different for patients with hormone receptor positive (HR+) cancer.

Applicants investigated the performance of the Set2,3 test (Endocrine Activity Index (EAI)) in the HR+ cancers from CALGB 9741. Applicants tests and its components predicted survival benefit from dose-intense chemotherapy and is the first example of a biomarker successfully predicting benefit from one chemotherapy regimen over another for patients with HR+ breast cancer. Applicants findings represents a novel paradigm.

In a first embodiment, there is provided method for treating cancer in a subject comprising:
(a) obtaining a biological sample from the subject;
(b) extracting a plurality of molecules from the biological sample;
(c) quantitating the expression levels of at least one hormone receptor gene(s) selected from the group consisting of ABAT, ADCY1, AZGP1, CA12, CD2, CD3D, DNAJC12, ESR1, KCNE4, MAPT, MRPS30, NAT1, NPY1R, PDZK1, QDPR, SCUBE2, SLC39A6, STC2 in the plurality of nucleic acids by detecting and quantitating one or more RNA transcripts associated with the at least one hormone receptor gene;
(d) quantitating the expression levels of at least one reference gene selected from the group consisting of AK2, APPBP2, ATP5J2, DARS, LDHA, TRIM2, UBE2Z, UGP2, VDAC2, WIPF2 in the plurality of nucleic acids by detecting and quantitating one or more RNA transcripts associated with the at least one reference gene;
(e) calculating a cut-off level by the formula

$$Cutoff = \frac{{\sum }_{i = 2}^{18} {T}_{i}}{i} - \frac{{\sum }_{j = 1}^{10} {R}_{j}}{j} + 2$$

where Tᵢ is the expression of the ith of the set of genes and Rⱼ the expression of the jth of the set of reference genes; and
(f) administering a dose-intense chemotherapy regimen to a subject with a cut-off less than 0.75, or administering a conventional chemotherapy regimen or other anti-cancer therapy to a subject with a cut-off level equal or greater than 0.75.

In some aspects, the method further comprises determining a molecular subtype of the biological sample to obtain an RNA4 risk score. In some aspects, the determining the molecular subtype of the biological sample comprises quantitating the expression levels of one or more of ESR1, PGR, ERBB2, and AURKA in the plurality of nucleic acids by detecting and quantitating one or more RNA transcripts associated with the molecular subtype. In specific aspects, the expression levels of the one or more of the ESR1, PGR, ERBB2, and AURKA genes are normalized by subtracting the mean of the 10 reference genes and adding the constant of 10. In some aspects, the RNA4 risk score is calculated as the sum of risk scores for each of the expression levels of ESR1, PGR, ERBB2, and AURKA.

In certain aspects, the cancer is breast cancer, such as metastatic breast cancer.

In additional aspects, the method further comprises determining whether the subject is positive for estrogen receptors (ER+), progesterone receptors (PR+) or both (ER+/PR+). In some aspects, the method further comprises determining a pT risk score. For example, the pT risk score is calculated from largest pathological tumor dimension and assigned score of zero if ≤10 mm, linearly scaled to increase 0.1 units per 1 mm increase in tumor dimension in the range 0-3.0 if measuring 11-39 mm, assigned risk score 3.0 if ≥40 mm, and assigned risk score 3.0 if the tumor otherwise has stage category pT4.

In some aspects, the method further comprises determining a pN risk score. In some aspects, the pN risk score is calculated from the number of involved lymph nodes by the formula 0.5 units per node. In certain aspects, the pN risk score is 5.0 if 10 or more nodes are involved or stage category pN3. In some aspects, a lymph node reported as isolated tumor cells (pNOi+) is considered to be negative for the pN risk score.

In certain aspects, the method further comprises determining a second cut-off level by the formula:

$$EAI Index Cutoff = 0.75 \times ( \frac{{\sum }_{i = 1}^{18} Ti}{i} - \frac{{\sum }_{j = 1}^{10} Rj}{j} + 2 ) + 0.51 \times ( 11 - ( pT + pN + RNA 4 ) ] \times 4 / 11 ) .$$

In certain aspects, the method further comprises administering a dose-intense chemotherapy regimen to a subject determined to have an EAI Index cutoff less than 1.0 (e.g., less than 0.95, 0.9, 0.85, 0.8, or 0.75), or administering a conventional chemotherapy regimen or other anti-cancer therapy to a subject determined to have an EAI Index cutoff greater than or equal to 1.0 (e.g., greater than 1.1, 1.2, 1.3, 1.4, 1.5, 1.6, 1.7, 1.8, 1.9, 2.0, 2.1, 2.2, 2.3, 2.4, or 2.5). In some aspects, the method further comprises administering a dose-intense chemotherapy regimen to a subject determined to have an EAI Index cutoff less than 2.1 (e.g., less than 2.0, 1.9, 1.8, 1.7, 1.6, 1.5, 1.4, 1.3, 1.2, or 1.1), or administering a conventional chemotherapy regimen to a subject determined to have an EAT Index cutoff greater than or equal to 2.1 (e.g., greater than 2.2, 2.3, 2.4, or 2.5).

In certain aspects, the dose intense chemotherapy regimen comprises administering 20 mg/m² to 250 mg/m² (e.g., 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 mg/m²) of an anthracycline and/or 30 mg/m² to 300 mg/m² (e.g., 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 mg/m²) of a taxane every one week, two weeks, or three weeks (e.g., two weeks). In certain aspects, the dose intense chemotherapy regimen comprises administering 20 mg/m² to 100 mg/m² of an anthracycline and/or 150 mg/m² to 200 mg/m² of a taxane every two weeks. In particular aspects, the dose intense chemotherapy regimen comprises administering 60 mg/m² of an anthracycline in combination with 175 mg/m² of a taxane every two weeks. In some aspects, at least 3 cycles of the dose intense chemotherapy regimen are administered. In certain aspects, 3, 4, 5, 6, 7, or 8 cycles of dose intense chemotherapy are administered, such as 4 or 6 cycles. In some aspects, there is a resting phase between cycles. In some aspects, the anthracycline is doxorubicin or epirubicin.

In specific aspects, the taxane is paclitaxel, docetaxel, cabazitaxel, or abraxane. In particular aspects, the taxane is paclitaxel. In some aspects, the dose intense chemotherapy further comprises administering cyclophosphamide. In some aspects, the anthracycline, cyclophosphamide, and/or taxane are administered sequentially. In certain aspects, the anthracycline, cyclophosphamide, and/or taxane are administered concurrently. In some aspects, the cyclophosphamide is administered at a dose of 250 mg/m²to 1000 mg/m² (e.g., 300, 325, 350, 375, 400, 425, 450, 475, 500, 525, 550, 575, 600, 625, 650, 675, 700, 725, 750, 775, 800, 825, 850, 875, 900, 925, 950, 975, or 1000 mg/m²). For example, the cyclophosphamide is administered at a dose of 600 mg/m².

In particular aspects, the anthracycline, taxane, and cyclophosphamide are administered every two weeks for four or six cycles. For example, the doxorubicin is administered at 60 mg/mg² intravenously, cyclophosphamide at 600 mg/mg² intravenously, and paclitaxel at 175 mg/m² intravenously every 2 weeks. In other aspects, paclitaxel is administered at 80 mg/m² intravenously weekly. In some aspects, docetaxel is administered at 35 mg/m² intravenously weekly. In other aspects, docetaxel is not administered in a dose intense regimen. In some aspects, cabazitaxel is administered at 20-25 mg/m² intravenously every 3 weeks. In certain aspects, abrazaxane is administered at 80-300 mg/m² intravenously every 3 weeks.

In some aspects, for a conventional or standard chemotherapy regimen, the anthracycline, cyclophosphamide and/or taxane is administered at a longer interval as compared to the dose intense regimen, such as every 3 weeks vs every 2 weeks. In some aspects, is the cutoff level is greater than 0.75 (e.g., greater than 0.8, 0.85, 0.9, 0.95, 1, 1.1, 1.2, 1.3, 1.4, 1.5, 1.6, 1.7, 1.8, 1.9, or 2.0), for a conventional chemotherapy regimen For example, docetaxel may be administered every 3 weeks at 20-200 mg/m², such as about 100 mg/m².

In some aspects, the dose intense chemotherapy regimen comprises administering to the subject an amount of 30 mg/m² to 250 mg/m² of a taxane and/or 25 mg/m² to 200 mg/m² of an anthracycline at weekly intervals for at least 6 treatments. In certain aspects, the dose intense chemotherapy regimen comprises administering to the subject an amount of 30 mg/m² to 250 mg/m² of a taxane and/or 25 mg/m² to 200 mg/m² of an anthracycline every two weeks for at least 3 cycles. In some aspects, the taxane is selected from the group consisting of paclitaxel, docetaxel, cabazitaxel, or abraxane.

In certain aspects, if the cut-off level is less than 2.1 the subject is administered a dose-intense paclitaxel chemotherapy regimen. In some aspects, if the cut-off level is equal then or greater than 2.1, then the subject is not administered. In certain aspects, if the cut-off level is equal than or greater than 2.1, then either an anthracycline chemotherapy or a taxane chemotherapy is administered on a conventional schedule. In some aspects, if the cut-off level is equal to or greater than 2.1, then a chemotherapy selected from the group consisting of paclitaxel, docetaxel, cabazitaxel, abraxane, doxorubicin, cyclophosphamide, or anthracycline is administered on a conventional schedule. In certain aspects, the conventional schedule comprises administering to the subject an amount of 30 mg/m² to 250 mg/m² of a taxane and/or 25 mg/m² to 200 mg/m² of an anthracycline every three weeks for at least 3 cycles.

In some aspects, the biological sample is a tumor biopsy. In certain aspects, the biological sample is formalin-fixed or a parafilm-embedded tissue biopsy.

In certain aspects, quantitating comprises performing reverse transcription-quantitative real-time PCR (RT-qPCR), microarray analysis, amplification-free nucleic acid analysis, picodroplet targeting and reverse transcription, or RNA sequencing. In some aspects, quantifying comprises performing RT-qPCR. In certain aspects, quantifying comprises performing nanostring nCounter. In some aspects, quantifying comprising performing targeted RNA sequencing. In some aspects, the microarray probe sequence comprise one or more of the probes of SEQ ID Nos:1-11 (SLC39A6), SEQ ID Nos:12-22 (STC2), SEQ ID Nos:13-33 (CA12), SEQ ID Nos:34-44 (PDZK1), SEQ ID Nos:45-55 (NPY1R), SEQ ID Nos:56-66 (CD2), SEQ ID Nos:67-77 (QDPR), SEQ ID Nos:78-88 (AZGP1), SEQ ID Nos:89-99 (ADCY1), SEQ ID Nos:100-110 (CD3D), SEQ ID Nos:111-121 (NAT1), SEQ ID Nos:122-132 (MRPS30), SEQ ID Nos:133-143 (DNAJC12), SEQ ID Nos:144-154 (SCUBE2), SEQ ID Nos:155-165 (MAPT), SEQ ID Nos:166-176 (ABAT), SEQ ID Nos:177-187 (KCNE2), SEQ ID Nos:188-198 (ESR1), SEQ ID Nos:199-209 (LDHA), SEQ ID Nos: 210-220 (ATP5J2), SEQ ID Nos:221-231 (VDAC2), SEQ ID Nos:232-242 (DARS), SEQ JD Nos:243-253 (UGP2), SEQ ID Nos:254-264 (UBE2Z), SEQ ID Nos:265-275 (AK2), SEQ ID Nos:276-286 (WIPF2), SEQ ID Nos:287-297 (APPBP2), SEQ ID Nos:298-308 (TRIM2), SEQ ID Nos:309-319 (ERBB2), SEQ ID Nos:(320-330), and SEQ ID Nos:331-341 (PGR) or one or more probes with at least 90%, 95%, 96%, 97%, 98%, or 99% sequence similarity to probes of SEQ ID Nos:1-341, or one or more probes of locations disclosed in Table 12. In certain aspects, the QGP probe sequence may comprise one or more probes of SEQ ID NO:342 (WIPF2), SEQ ID NO:343 (CA2), SEQ ID NO:344 (SCUBE2), SEQ ID NO:345 (ERBB2), SEQ ID NO:346 (TRIM2), SEQ ID NO:347 (ESR1), SEQ ID NO:348 (SLC39A6), SEQ ID NO:349 (MRPS30), SEQ ID NO:350 (STC2), SEQ ID NO:351 (PDZK1), SEQ ID NO:352 (NAT1), SEQ ID NO:353 (ADCY1), SEQ ID NO:354 (MAPT), SEQ ID NO:355 (LDHA), SEQ ID NO:356 (CD2), SEQ ID NO:357 (UBE2Z), SEQ ID NO:358 (AK2), SEQ ID NO:359 (AURKA), SEQ ID NO:360 (AZGP1), SEQ ID NO:361 (PGR), SEQ ID NO:362 (KCNE2), SEQ ID NO:363 (ABPPBP2), SEQ ID NO:364 (CD3D), SEQ ID NO:365 (DNAJC12), SEQ ID NO:366 (NPY1R), SEQ ID NO:367 (ABAT), SEQ ID NO:368 (UGP2), SEQ ID NO:369 (ATP5J2), SEQ ID NO:370 (QDPR), SEQ ID NO:371 (VDAC2), and SEQ ID NO:372 (DARS) or one or more probes with at least 90%, 95%, 96%, 97%, 98%, or 99% sequence similarity to probes of SEQ ID Nos:342-372, or one or more probes of locations disclosed in Table 12.

In certain aspects, targeted RNA sequencing may comprise the use of one or amplicon sequences of SEQ ID NO:373 (SLC39A6), SEQ ID NO:374 (STC2), SEQ ID NO:375 (CA12), SEQ ID NO:376 (PDZK1), SEQ ID NO:377 (NPY1R), SEQ ID NO:378 (CD2), SEQ ID NO:379 (QDPR), SEQ ID NO:380 (AZGP1), SEQ ID NO:381 (ADCY1), SEQ ID NO:382 (CD3D), SEQ ID NO:383 (NAT1), SEQ ID NO:384 (MRPS30), SEQ ID NO:385 (DNAJC12), SEQ ID NO:386 (SCUBE2), SEQ ID NO:387 (PGR), SEQ ID NO:388 (MAPT), SEQ ID NO:389 (ABAT), SEQ ID NO:390 (KCNE4), SEQ ID NO:391 (ERBB2), SEQ ID NO:392 (AURKA), SEQ ID NO:393 (LDHA), SEQ ID NO:394 (ATP5J2), SEQ ID NO:395 (VDAC2), SEQ ID NO:396 (DARS), SEQ ID NO:397 (UGP2), SEQ ID NO:398 (AK2), SEQ ID NO:399 (UBE2Z), SEQ ID NO:400 (WIPF2), SEQ ID NO:401 (APPBP2), SEQ ID NO:402 (TRIM2), and SEQ ID NO:403 (ESR1) or one or more amplicons with at least 90%, 95%, 96%, 97%, 98%, or 99% sequence similarity to probes of SEQ ID Nos:373-403, or one or more amplicons of locations disclosed in Table 12

In certain aspects, the method further comprises administering an additional anti-cancer therapy. In some aspects, the anti-cancer therapy increases the effectiveness of the dose-intense chemotherapy. In certain aspects, the additional anti-cancer therapy is a radiation therapy, hormone therapy, immunotherapy or cytokine therapy. In some aspects, the additional anti-cancer therapy is an immunotherapy, such as an immune checkpoint inhibitor. For example, the immune checkpoint inhibitor is an inhibitor of CTLA-4, PD-1, PD-L1, PD-L2, LAG-3, BTLA, B7H3, B7H4, TIM3, KIR, or A2aR. In particular aspects, the immune checkpoint inhibitor is an anti-CTLA-4, anti-PD-1, or anti-PD-L1 antibody. In some aspects, the anti-PDL1 antibody is atezolizumab, durvalumab, and avelumab. In certain aspects, the anti-PD1 antibody is nivolumab, pembrolizumab, pidilizumab, KEYTRUDA®, AMP-514, REGN2810, CT-01i, BMS 936559, MPDL3280A or AMP-224. In some aspects, the anti-CTLA-4 antibody is tremelimumab, YERVOY®, or ipilimumab.

In one embodiment, the present disclosure provides methods for predicting effectiveness of dose-intense chemotherapy regimens on a subject, the method comprising the steps of:
determining whether the subject is positive for estrogen receptors (ER+), progesterone receptors (PR+) or both (ER+/PR+) by:
obtaining a biological sample from the subject;
extracting a plurality of molecules from the biological sample;
performing an assay that:
quantitates the expression levels of at least one hormone receptor gene(s) selected from the group consisting of ABAT, ADCY1, AZGP1, CA12, CD2, CD3D, DNAJC12, ESR1, KCNE4, MAPT, MRPS30, NAT1, NPY1R, PDZK1, QDPR, SCUBE2, SLC39A6, STC2 in the plurality of nucleic acids by detecting and quantitating one or more RNA transcripts associated with the at least one hormone receptor gene;
quantitates the expression levels of at least one reference gene selected from the group consisting of AK2, APPBP2, ATP5J2, DARS, LDHA, TRIM2, UBE2Z, UGP2, VDAC2, WIPF2 in the plurality of nucleic acids by detecting and quantitating one or more RNA transcripts associated with the at least one reference gene;
provides the expression levels of the detected genes with the formula expression levels of

$$ER + , PR + , or both transcripts = \frac{{\sum }_{i = 1}^{18} Ti}{i} - \frac{{\sum }_{j = 1}^{10} Rj}{j} + 2$$

where Tᵢ is the expression of the ith of the set of genes and Rⱼ the expression of the jth of the set of reference genes;
if the subject has a cut-off level that is lesser than 1.0 then internally administering a dose-intense chemotherapy regimen to the patient;
if the subject has a cut-off level that is equal then or greater than 1.0, then administering a conventional chemotherapy regiment to the patient or not administering any chemotherapy to the patient.

In some aspects, the performing of the assay further comprises determining a molecular subtype of the biological sample. In certain aspects, the determining the molecular subtype of the biological sample comprises quantitating the expression levels of one or more of ESR1, PGR, ERBB2, and AURKA in the plurality of nucleic acids by detecting and quantitating one or more RNA transcripts associated with the molecular subtype. In some aspects, the expression levels of the one or more of the ESR1, PGR, ERBB2, and AURKA genes are normalized by subtracting the mean of the 10 reference genes and adding the constant of 10.

In certain aspects, the cancer is breast cancer, such as metastatic breast cancer.

In particular aspects, the performing of the assay further comprises determining a pT risk score. In some aspects, the pT risk score is calculated from largest pathological tumor dimension and assigned score of zero if ≤10 mm, linearly scaled to the range 0-3.0 if measuring 11-39 mm, assigned risk score 3.0 if ≥40 mm, and assigned risk score 3.0 if the tumor otherwise has stage category pT4. In certain aspects, the performing of the assay further comprises determining a pN risk score. In some aspects, the pN risk score is calculated from the number of involved lymph nodes (0.5 units per node) and assigned score of 5.0 if 10 or more nodes are involved or otherwise stage category pN3. Any lymph node reported as isolated tumor cells (pN0i+) is considered to be negative for this score.

In additional aspects, the method further comprises determining a second cut-off level by the formula:

$$EAI Index Cutoff = 0.75 \times ( \frac{{\sum }_{i = 1}^{18} Ti}{i} - \frac{{\sum }_{j = 1}^{10} Rj}{j} + 2 ) + 0.51 \times ( 11 - ( pT + pN + RNA 4 ) ] \times 4 / 11 ) .$$

In some aspects, the dose intense chemotherapy regimen comprises administering to the subject an amount of 25 mg/m² b.i.d to 350 mg/m², 50 mg/m² b.i.d to 300 mg/m² b.i.d, 75 mg/m² b.i.d to 250 mg/m² b.i.d, or 160 mg/m² b.i.d to 250 mg/m² b.i.d (e.g., about 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, or 350 mg/m²) b.i.d of a taxane every week for a period of at least 6 weeks. In certain aspects, the dose intense chemotherapy regimen comprises administering to the subject an amount of 25 mg/m² b.i.d to 350 mg/m², 50 mg/m² b.i.d to 300 mg/m² b.i.d, 75 mg/m² b.i.d to 250 mg/m² b.i.d, or 160 mg/m² b.i.d to 250 mg/m² b.i.d (e.g., about 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, or 350 mg/m²) of a taxane every two weeks for a period of at least 6 weeks. In some aspects, the dose intense chemotherapy regimen comprises administering to the subject an amount of 25 mg/m² b.i.d to 350 mg/m², 50 mg/m² b.i.d to 300 mg/m² b.i.d, 75 mg/m² b.i.d to 250 mg/m2 b.i.d, or 160 mg/m² b.i.d to 250 mg/m² b.i.d (e.g., about 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, or 350 mg/m²) of a taxane every three weeks for a period of at least 6 weeks. In particular aspects, the taxane is selected from the group consisting of paclitaxel, docetaxel, cabazitaxel, or abraxane.

In some aspects, the dose intense chemotherapy regimen comprises administering to the subject an amount of 25 mg/m² to 350 mg/m², 50 mg/m² to 300 mg/m², 75 mg/m² to 250 mg/m², or 160 mg/m² to 250 mg/m² (e.g., about 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, or 350 mg/m²) of a taxane every week for a period of at least 6 weeks. In certain aspects, the dose intense chemotherapy regimen comprises administering to the subject an amount of 25 mg/m² to 350 mg/m², 50 mg/m² to 300 mg/m², 75 mg/m² to 250 mg/m², or 160 mg/m² to 250 mg/m² (e.g., about 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, or 350 mg/m²) of a taxane every two weeks for a period of at least 6 weeks. In some aspects, the dose intense chemotherapy regimen comprises administering to the subject an amount of 25 mg/m² to 350 mg/m², 50 mg/m² to 300 mg/m², 75 mg/m² to 250 mg/m², or 160 mg/m² to 250 mg/m² (e.g., about 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, or 350 mg/m²) of a taxane every three weeks for a period of at least 6 weeks. In particular aspects, the taxane is selected from the group consisting of paclitaxel, docetaxel, cabazitaxel, or abraxane.

For example, a paclitaxel weekly dose may comprise 60-120 mg/m² (e.g., 80 mg/m²). In some aspects, a paclitaxel dose-dense 2-weekly dose may be administered at 160-250 mg/m² (e.g., 175 mg/m²). In certain aspects, a paclitaxel 3-weekly dose may be administered at 160-250 mg/m² (e.g., 175 mg/m²). In some aspects, a docetaxel weekly dose may comprise 35 mg/m². In some aspects, a docetaxel 3-weekly may be administered at 80-120 mg/m² (e.g., 100 mg/m²). In certain aspects, a doxorubicin dose-dense 2-weekly dose may be administered at 40-80 mg/m² (e.g., 60 mg/m²). In certain aspects, a doxorubicin 3-weekly dose may be administered at 40-80 mg/m² (e.g., 60 mg/m²). In some aspects, an epirubicin dose-dense 2-weekly dose may be administered at 30-120 mg/m² (e.g., 60-100 mg/m²). In certain aspects, an epirubicin 3-weekly dose may be administered at 60-120 mg/m² (e.g., 75-100 mg/m²). In some aspects, a cyclophosphamide dose-dense 2-weekly dose may be administered at 400-750 mg/m² (e.g., 500 or 600 mg/m²). In some aspects, a cyclophosphamide 3-weekly dose may be administered at 400-750 mg/m² (usual is 500 or 600 mg/m²). In certain aspects, a fluorouracil 3-weekly dose may be administered at 400-750 mg/m² (e.g., 500-600 mg/m2).

In certain aspects, the dose intense chemotherapy regimen comprises administering 20 mg/m² to 250 mg/m² (e.g., 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 mg/m²) of an anthracycline and/or 30 mg/m2 to 300 mg/m² (e.g., 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 mg/m²) of a taxane every one week, two weeks, or three weeks (e.g., two weeks). In certain aspects, the dose intense chemotherapy regimen comprises administering 20 mg/m² to 100 mg/m² of an anthracycline and/or 150 mg/m² to 200 mg/m² of a taxane every two weeks. In particular aspects, the dose intense chemotherapy regimen comprises administering 60 mg/m² of an anthracycline in combination with 175 mg/m² of a taxane every two weeks. In some aspects, at least 3 cycles of the dose intense chemotherapy regimen are administered. In certain aspects, 3, 4, 5, 6, 7, or 8 cycles of dose intense chemotherapy are administered, such as 4 or 6 cycles. In some aspects, there is a resting phase between cycles. In some aspects, the anthracycline is doxorubicin or epirubicin.

In specific aspects, the taxane is paclitaxel, docetaxel, cabazitaxel, or abraxane. In particular aspects, the taxane is paclitaxel. In some aspects, the dose intense chemotherapy further comprises administering cyclophosphamide. In some aspects, the anthracycline, cyclophosphamide, and/or taxane are administered sequentially. In certain aspects, the anthracycline, cyclophosphamide, and/or taxane are administered concurrently. In some aspects, the cyclophosphamide is administered at a dose of 250 mg/m²to 1000 mg/m² (e.g., 300, 325, 350, 375, 400, 425, 450, 475, 500, 525, 550, 575, 600, 625, 650, 675, 700, 725, 750, 775, 800, 825, 850, 875, 900, 925, 950, 975, or 1000 mg/m²). For example, the cyclophosphamide is administered at a dose of 600 mg/m².

In particular aspects, the anthracycline, taxane, and cyclophosphamide are administered every two weeks for four or six cycles. For example, the doxorubicin is administered at 60 mg/mg² intravenously, cyclophosphamide at 600 mg/mg² intravenously, and paclitaxel at 175 mg/m² intravenously every 2 weeks. In other aspects, paclitaxel is administered at 80 mg/m2 intravenously weekly. In some aspects, docetaxel is administered at 35 mg/m² intravenously weekly. In some aspects, cabazitaxel is administered at 20-25 mg/m² intravenously every 3 weeks. In certain aspects, abrazaxane is administered at 80-300 mg/m² intravenously every 3 weeks.

In some aspects, for a conventional or standard chemotherapy dose, the taxane is administered every 3 weeks. For example, docetaxel every 3 weeks may be administered at 20-200 mg/m², such as about 100 mg/m².

In some aspects, if the cut-off level is lesser than 2.1, then internally administering a dose-intense paclitaxel chemotherapy regimen to the patient.

In certain aspects, the dose intense chemotherapy regimen comprises administering to the subject an amount of 25 mg/m² b.i.d to 200 mg/m² (e.g., 25, 30, 35, 40, 45, 50, 55, 60, 65, 70, 75, 80, 85, 90, 100, 110, 120, 130, 140, 150, 160, 170, 180, 190, or 200 mg/m²) b.i.d of an anthracycline every week for a period of at least 6 weeks. In some aspects, the dose intense chemotherapy regimen comprises administering to the subject an amount of 50 mg/m² b.i.d to 150 mg/m² b.i.d of an anthracycline every two weeks for a period of at least 6 weeks. In some aspects, the dose intense chemotherapy regimen comprises administering to the subject an amount of 75 mg/m² b.i.d to 100 mg/m² b.i.d of an anthracycline every three weeks for a period of at least 6 weeks.

In some aspects, if the cut-off level is equal then or greater than 2.1, then a dose-intense chemotherapy regimen is not administered to the subject. In certain aspects, if the cut-off level is equal then or greater than 2.1, then either an anthracycline chemotherapy or a taxane chemotherapy is administered on a conventional schedule. In some aspects, if the cut-off level is equal then or greater than 2.1, then a chemotherapy selected from the group consisting of paclitaxel, docetaxel, cabazitaxel, abraxane, doxorubicin, cyclophosphamide, or anthracycline is administered on a conventional schedule.

In certain aspects, the biological sample is a tumor biopsy. In some aspects, the biological sample is formalin-fixed or a paraffin-embedded tissue biopsy. In specific aspects, the assay that quantitates the expression levels of the plurality of nucleic acids comprises performing reverse transcription-quantitative real-time PCR (RT-qPCR), microarray analysis, amplification-free nucleic acid analysis, picodroplet targeting and reverse transcription, or RNA sequencing.

In some aspects, the microarray probe sequence comprise one or more of the probes of SEQ ID Nos:1-11 (SLC39A6), SEQ ID Nos:12-22 (STC2), SEQ ID Nos:13-33 (CA12), SEQ ID Nos:34-44 (PDZK1), SEQ JD Nos:45-55 (NPY1R), SEQ JD Nos:56-66 (CD2), SEQ ID Nos:67-77 (QDPR), SEQ ID Nos:78-88 (AZGP1), SEQ ID Nos:89-99 (ADCY1), SEQ ID Nos:100-110 (CD3D), SEQ ID Nos:111-121 (NAT1), SEQ ID Nos:122-132 (MRPS30), SEQ ID Nos:133-143 (DNAJC12), SEQ ID Nos:144-154 (SCUBE2), SEQ ID Nos: 155-165 (MAPT), SEQ ID Nos:166-176 (ABAT), SEQ ID Nos:177-187 (KCNE2), SEQ ID Nos:188-198 (ESR1), SEQ ID Nos:199-209 (LDHA), SEQ ID Nos: 210-220 (ATP5J2), SEQ ID Nos:221-231 (VDAC2), SEQ ID Nos:232-242 (DARS), SEQ ID Nos:243-253 (UGP2), SEQ ID Nos:254-264 (UBE2Z), SEQ ID Nos:265-275 (AK2), SEQ ID Nos:276-286 (WIPF2), SEQ ID Nos:287-297 (APPBP2), SEQ ID Nos:298-308 (TRIM2), SEQ ID Nos:309-319 (ERBB2), SEQ ID Nos:(320-330), and SEQ ID Nos:331-341 (PGR) or one or more probes with at least 90%, 95%, 96%, 97%, 98%, or 99% sequence similarity to probes of SEQ ID Nos:1-341, or one or more probes of locations disclosed in Table 12. In certain aspects, the QGP probe sequence may comprise one or more probes of SEQ ID NO:342 (WIPF2), SEQ ID NO:343 (CA2), SEQ ID NO:344 (SCUBE2), SEQ ID NO:345 (ERBB2), SEQ ID NO:346 (TRIM2), SEQ ID NO:347 (ESR1), SEQ ID NO:348 (SLC39A6), SEQ ID NO:349 (MRPS30), SEQ ID NO:350 (STC2), SEQ ID NO:351 (PDZK1), SEQ ID NO:352 (NAT1), SEQ ID NO:353 (ADCY1), SEQ ID NO:354 (MAPT), SEQ ID NO:355 (LDHA), SEQ ID NO:356 (CD2), SEQ ID NO:357 (UBE2Z), SEQ ID NO:358 (AK2), SEQ ID NO:359 (AURKA), SEQ ID NO:360 (AZGP1), SEQ ID NO:361 (PGR), SEQ ID NO:362 (KCNE2), SEQ ID NO:363 (ABPPBP2), SEQ ID NO:364 (CD3D), SEQ ID NO:365 (DNAJC12), SEQ ID NO:366 (NPY1R), SEQ ID NO:367 (ABAT), SEQ ID NO:368 (UGP2), SEQ ID NO:369 (ATP5J2), SEQ ID NO:370 (QDPR), SEQ ID NO:371 (VDAC2), and SEQ ID NO:372 (DARS) or one or more probes with at least 90%, 95%, 96%, 97%, 98%, or 99% sequence similarity to probes of SEQ ID Nos:342-372, or one or more probes of locations disclosed in Table 12.

In certain aspects, targeted RNA sequencing may comprise the use of one or amplicon sequences of SEQ ID NO:373 (SLC39A6), SEQ ID NO:374 (STC2), SEQ ID NO:375 (CA12), SEQ ID NO:376 (PDZK1), SEQ ID NO:377 (NPY1R), SEQ ID NO:378 (CD2), SEQ ID NO:379 (QDPR), SEQ ID NO:380 (AZGP1), SEQ ID NO:381 (ADCY1), SEQ ID NO:382 (CD3D), SEQ ID NO:383 (NAT1), SEQ ID NO:384 (MRPS30), SEQ ID NO:385 (DNAJC12), SEQ ID NO:386 (SCUBE2), SEQ ID NO:387 (PGR), SEQ ID NO:388 (MAPT), SEQ ID NO:389 (ABAT), SEQ ID NO:390 (KCNE4), SEQ ID NO:391 (ERBB2), SEQ ID NO:392 (AURKA), SEQ ID NO:393 (LDHA), SEQ ID NO:394 (ATP5J2), SEQ ID NO:395 (VDAC2), SEQ ID NO:396 (DARS), SEQ ID NO:397 (UGP2), SEQ ID NO:398 (AK2), SEQ ID NO:399 (UBE2Z), SEQ ID NO:400 (WIPF2), SEQ ID NO:401 (APPBP2), SEQ ID NO:402 (TRIM2), and SEQ ID NO:403 (ESR1) or one or more amplicons with at least 90%, 95%, 96%, 97%, 98%, or 99% sequence similarity to probes of SEQ ID Nos:373-403, or one or more amplicons of locations disclosed in Table 12

In certain aspects, the method further comprises administering an additional anti-cancer therapy. In some aspects, the anti-cancer therapy increases the effectiveness of the dose-intense chemotherapy. In certain aspects, the additional anti-cancer therapy is a radiation therapy, hormone therapy, immunotherapy or cytokine therapy. In some aspects, the additional anti-cancer therapy is an immunotherapy, such as an immune checkpoint inhibitor. For example, the immune checkpoint inhibitor is an inhibitor of CTLA-4, PD-1, PD-L1, PD-L2, LAG-3, BTLA, B7H3, B7H4, TIM3, KIR, or A2aR. In particular aspects, the immune checkpoint inhibitor is an anti-CTLA-4, anti-PD-1, or anti-PD-L1 antibody. In some aspects, the anti-PDL1 antibody is atezolizumab, durvalumab, and avelumab. In certain aspects, the anti-PD1 antibody is nivolumab, pembrolizumab, pidilizumab, KEYTRUDA®, AMP-514, REGN2810, CT-011, BMS 936559, MPDL3280A or AMP-224. In some aspects, the anti-CTLA-4 antibody is tremelimumab, YERVOY®, or ipilimumab.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Schematic of the study population from the study of Set2,3 index and Set_{ER/PR} in the CALGB 9741 study.

FIGS. 2A-2D: depict a long-term survival analysis of treatments in all patients treated in the CALGB 9741 (C9741) trial according to ER status. Survival analysis of treatments in C9741 according to ER status. (FIG. 2A) Kaplan-Meier plot of Disease-Free Survival in ER-positive population from C9741 (overall study population) showing survival curves for the dose-intense (dose-dense) versus the conventionally dosed (Q3-weekly) treatment arms. There was a significant benefit in disease-free survival (DFS) after dose-dense chemotherapy in the patients with ER-positive cancer, with hazard ratio 0.80 (95% confidence interval 0.65 to 0.98) and p value of 0.029. (FIG. 2B) Kaplan-Meier plot of Overall Survival in ER-positive population from C9741 (overall study population) showing survival curves for the dose-intense (dose-dense) versus the conventionally dosed (Q3-weekly) treatment arms. There was a near-significant trend favoring longer overall survival (OS) after dose-dense chemotherapy in the patients with ER-positive cancer, with hazard ratio 0.85 (95% confidence interval 0.68 to 1.06) and p value of 0.14. (FIG. 2C) Kaplan-Meier plot of Disease-Free Survival in ER-negative population from C9741 (overall study population) showing survival curves for the dose-intense (dose-dense) versus the conventionally dosed (Q3-weekly) treatment arms. There was a significant benefit in disease-free survival (DFS) after dose-dense chemotherapy in the patients with ER-negative cancer, with hazard ratio 0.70 (95% confidence interval 0.55 to 0.90) and p value of 0.005. (FIG. 2D) Kaplan-Meier plot of Overall Survival in ER-negative population from C9741 (overall study population) showing survival curves for the dose-intense (dose-dense) versus the conventionally dosed (Q3-weekly) treatment arms. There was a significant benefit in overall survival (OS) after dose-dense chemotherapy in the patients with ER-negative cancer, with hazard ratio 0.72 (95% confidence interval 0.55 to 0.93) and p value of 0.013.

FIG. 3A-3B: Prognosis related to the combined results of Set2,3 (also known as Endocrine Activity Index (EAI)) and ROR-PT score, and the association of Set2,3 and ROR-PT score with intrinsic subtype in the ER-positive population from the CALGB 9741 (C9741) trial. (FIG. 3A) Kaplan-Meier plot of Overall Survival in ER-positive population from C9741 according to the combined results of Set2,3 (also known as Endocrine Activity Index (EAI)) and ROR-PT score. There was significant prognostic information (log rank p value<0.001) with most of this information from the different survival outcomes according to whether the ER-positive breast cancer had high Set2,3 or low Set2,3 status. (FIG. 3B) Frequency bar charts of the percent of each intrinsic subtype within subsets defined by SET2,3 and ROR-PT categories. Most luminal A cancers (blue) had low ROR-PT score, but they were similarly distributed between high SET2,3 and low SET2,3 categories. Most luminal B cancers (orange) had high ROR-PT score, but they were similarly distributed between high SET2,3 and low SET2,3 categories. HER2-enriched (gray) and basal-like (yellow) cancers were mostly high ROR-PT and almost all had low SET2,3.

FIG. 4: Kaplan-Meier plot of Overall Survival of subjects in the CALGB 9741 trial with ER-positive breast cancer according to whether the tumor had low Set2,3 (also known as Endocrine Activity Index (EAI)) or high Set2,3 (low and high defined by the cut point of 2.10). There was a significant benefit in overall survival (OS) for the patients with high Set2,3, with hazard ratio 0.38 (95% confidence interval 0.27 to 0.54) and p value of <0.001. The figure also shows the estimated 5-year and 10-year overall survival (OS) for the patients with high Set2,3 versus low Set2,3 status.

FIGS. 5A-5B: Kaplan Meier plots of disease-free survival (DFS) of patients with ER-positive breast cancer in the CALGB 9741 trial demonstrate the predictive interaction between dose-intense chemotherapy and SET2,3 index using pre-defined cut point of 2.10. (FIGS. 5A-B) Kaplan-Meier plots of disease-free survival (DFS) by treatment arm (dose-dense Q2-weekly versus conventional Q3-weekly, and concurrent AC versus sequential A→C treatments) in patients with (FIG. 5A) Low SET2,3 cancer (≤2.10), or (FIG. 5B) High SET2,3 cancer (>2.10). Patients with breast cancer that had low SET2,3 index had better DFS outcomes from the most intensive concurrent dose-dense treatments (ddAC/T), as shown in FIG. 5A.

FIG. 6: Hazard function plot to demonstrate the relative benefit from dose-intense (Q2-weekly) versus conventional dosing (Q3-weekly) of chemotherapy on Overall Survival according to the value of the SET_{ER,PR} index measured from ER-positive breast cancers in the C9741 trial. There was relative benefit from the dose-intense chemotherapy treatment in patients whose cancer had lower SET_{ER,PR} index. SET_{ER,PR} index is a component of SET2,3 index and was noted to carry all of the predictive information that was observed with SET2,3 index, whereas BPI, the other component, did not predict survival benefit from dose-intense chemotherapy treatments.

FIGS. 7A-7B: Kaplan Meier plots of Overall Survival (OS) of patients with ER-positive breast cancer in the CALGB 9741 trial demonstrate the predictive interaction between dose-intense chemotherapy and SET_{ER,PR} index using the cut point of 0.75. (FIG. 7A) Overall survival when SET_{ER.PR} index in the breast cancer was less than 0.75. There was a significant benefit in overall survival (OS) after dose-intense chemotherapy when SET_{ER,PR} index in the breast cancer was less than 0.75, with hazard ratio 0.64 (95% confidence interval 0.42 to 0.99) and p value of 0.042. (FIG. 7B) Overall survival when SET_{ER,PR} index in the breast cancer was 0.75 or greater (CALGB 9741 trial). Patients did not benefit from dose-intense chemotherapy if the SET_{ER,PR} index in their breast cancer was 0.75 or greater: hazard ratio 0.95 (95% confidence interval 0.62 to 1.47) and p value of 0.825.

FIG. 8: Study design to evaluate the analytical validity of measurements of SET_{ER,PR} index from breast cancer samples using real-time quantitative polymerase chain reaction (RT qPCR), with biological replicates starting from the same aliquot of total RNA and technical replicates starting with unstained tumor tissue sections from the same tumor block. Second, to compare measurements using the QuantiGene Plex hybridization method (QGP) with measurements using real-time quantitative polymerase chain reaction (RT qPCR).

FIGS. 9A-9B: Analytical validity of measuring of SET_{ER,PR} index from breast cancer samples using real-time quantitative polymerase chain reaction (RT qPCR), and compared to the QuantiGene Plex hybridization method (QGP). (FIG. 9A): Left—Linear regression of RT qPCR measurements of SET_{ER/PR} index from 8 biological replicates from purified total RNA. Right—Extended Bland-Altman plot of standard deviation versus mean of the RT qPCR measurements of SET_{ER/PR} index from tissue sections of 41 FFPE samples tested in triplicate (technical replicates). (FIG. 9B): Comparison of RT qPCR measurements of SET_{ER,PR} index, versus (QGP) QuantiGene Plex hybridization method, from the 41 breast cancer samples (technical replicates). Left—Linear regression of SET_{ER/PR} index of the 41 samples showing the mean of three RT qPCR measurements versus a single referent QGP measurement. Right—Extended Bland-Altman plot of SET_{ER/PR} index showing the difference in measurements (mean of RT qPCR minus QGP) and the mean of RT qPCR measurements for 41 samples (technical replicates).

### DESCRIPTION OF ILLUSTRATIVE EMBODIMENTS

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The CALBG (Alliance) 9471 is a seminal phase III study evaluating how the schedule of adjuvant chemotherapy administration may influence outcomes for patients diagnosed with node-positive breast cancer. Using a 2×2 factorial design, C9741 randomized treatment with doxorubicin (A), cyclophosphamide (C) and paclitaxel (T). The two factors were (i) 2-weekly (dose intense; DD) versus 3-weekly administration and (ii) sequential (A→T→C) versus concurrent (AC→T) chemotherapy.¹ After a median follow-up of 3 years, dose-intense adjuvant chemotherapy, versus every-three-week schedules was associated with a 26% and 31% relative risk reduction for disease-free survival and overall survival, respectively.¹ No survival differences were observed between concurrent and sequential administration, and no interaction between density and sequence was identified.¹ A subsequent analysis at 6 years pointed to a significant interaction between ER status and dose-intense chemotherapy, with the benefits of the dose intense regimen vs. conventional regimens seen mostly among patients with ER-negative breast cancer.² However, subsequent meta-analyses have differed as to whether dose-intense versus standard dosing was associated with reductions in disease recurrence for both hormone receptor positive (ER-positive) and hormone receptor negative (ER-negative) breast cancer.^{3,4}

Dose intensity in chemotherapy may be calculated as the total dose received by a given patient per unit of time. Specifically, dose intensity can be a measure of chemotherapy delivery that looks at the amount of drug delivered per unit time (measured as mg/m2/wk). The relative dose intensity of a single-drug regimen can be expressed as the ratio of its dose intensities in test and standard regimens. Average relative dose intensity is calculated by averaging the relative dose intensity of each drug in a test regimen. A further refinement looks at the dose responses of individual drugs, calculates their unit dose intensities, and then combines them so that ‘summation dose intensity’ can be obtained for any drug regimen. It is possible to augment intensity by escalating the dose per cycle or by decreasing the interval between cycles, known as dose density. Dose-dense chemotherapy increases the dose intensity of the regimen by delivering standard-dose chemotherapy with shorter intervals between the treatment cycles. This approach can be used to compare and refine chemotherapy regimens in breast cancer. Higher dose intensity can be delivered by escalating the dose per-cycle or by reducing the intervals between cycles, known as dose density. The terms “dose intense” and “dose dense” are used interchangeably herein.

In general, chemotherapy is believed to be effective in proliferating cells and its consequence is cell death. The rationale for evaluating dose-density in C9741 was based on mathematical evidence that cytotoxic agents kill a fixed fraction of cancer cells per dose and recovery of the cancer population between doses is rapid due to low-volume Gompertzian growth kinetics.⁵ So, theoretically, high proliferation (associated with aggressive molecular subtype) would be associated with high kill rate and rapid regrowth. However, in a retrospective analysis of C9741, the Prosigna® test was performed using a research platform and neither the risk of recurrence score enhanced with measurements of proliferation and tumor size (ROR-PT score), nor the PAM50 intrinsic subtype predicted benefit from dose intense chemotherapy.⁶ Intrinsic subtype, defined from immunohistochemical stains, also did not predict benefit from dose-intense chemotherapy in another trial (GIM2).⁷ Actually, none of the contemporary genomic tests in clinical predicted survival benefit from a taxane-containing chemotherapy regimen in node-positive ER-positive breast cancer.^{6,8-13}

Applicants hypothesized and methodically validated an association between the level of endocrine-related activity in the cancer and ability of cells to survive and recover from various chemotherapy regimens. In particular, Applicants validated a specific and tailored methodology for predicting instances when each chemotherapy dose, then dose-intense chemotherapy would be effective on a patient when there is a low level of endocrine-related activity.

SET2,3 index (commercially developed as Endocrine Activity Index (EAI)) is a 31-gene expression assay that offers prognostic information for patients receiving endocrine therapy.¹⁴⁻¹⁶ It measures the 28-gene SET_{ER/PR} index of transcription related to estrogen and progesterone receptors, but excluding proliferation-related genes, as well as a baseline prognostic index (BPI) derived from pathologic tumor size, number of involved lymph nodes and molecular subtype by RNA4 (ESR1, PGR, ERBB2, and AURKA) wherein higher BPI represents less aggressive disease.^{14,17} High Endocrine Activity Index (EAI) scores (≥2.10) are associated with endocrine sensitivity and more favorable prognosis, and low Endocrine Activity Index (EAI) scores (<2.10) with endocrine therapy resistance and less favorable outcomes.¹⁴⁻¹⁶ The assay uses a customized multiplex RNA hybridization assay designed for formalin fixed paraffin embedded (FFPE) tumor tissues and is highly reproducible results within and between different laboratories.^{18,19} In prior studies, the SET_{ER/PR} index and BPI each added independent prognostic information to contemporary prognostic signatures, including the 70-gene MammaPrint (MP) and RS tests.^{15,18}

Here, Applicants describe the analyses of the study defined endpoints of disease-free survival (DFS) and overall survival (OS) after 12.3 years of median follow-up of C9741. Focusing on ER-positive breast cancer, Applicants then report the predictive and prognostic associations of the Endocrine Activity Index (EAI) index, comparing this with the available results from the proliferation-driven ROR-PT score and intrinsic subtypes.

### I. Definitions

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a hormonal therapy.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Prognosis” refers to a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression-free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis and/or cancer progression in a patient susceptible to or diagnosed with a cancer. Prognosis also includes prediction of favorable survival following cancer treatments, such as a conventional cancer therapy.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The terms “hormonal” and “endocrine” therapy or treatment are used interchangeably herein to refer to an agent which blocks the body's ability to produce a specific hormone (e.g., estrogen) or interferes with hormone action.

The term “determining an expression level” as used herein means the application of a gene specific reagent such as a probe, primer or antibody and/or a method to a sample, for example a sample of the subject and/or a control sample, for ascertaining or measuring quantitatively, semi-quantitatively or qualitatively the amount of a gene or genes, for example the amount of mRNA. For example, a level of a gene can be determined by a number of methods including for example immunoassays including for example immunohistochemistry, ELISA, Western blot, immunoprecipitation and the like, where a biomarker detection agent such as an antibody for example, a labeled antibody, specifically binds the biomarker and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR, serial analysis of gene expression (SAGE), Northern Blot, digital molecular barcoding technology, for example Nanostring:nCounter™ Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene®ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and for example for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

The term “sample” as used herein includes any biological specimen obtained from a patient. Samples include, without limitation, whole blood, plasma, serum, red blood cells, white blood cells (e.g., peripheral blood mononuclear cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by fine needle aspiration that is directed to a target, such as a tumor, or is random sampling of normal cells, such as periareolar), any other bodily fluid, a tissue sample (e.g., tumor tissue) such as a biopsy of a tumor (e.g., needle biopsy) or a lymph node (e.g., sentinel lymph node biopsy), and cellular extracts thereof. In some embodiments, the sample is whole blood or a fractional component thereof such as plasma, serum, or a cell pellet. In some embodiments, the sample is a formalin fixed paraffin embedded (FFPE) tumor tissue sample, e.g., from a solid tumor of the breast.

A “biopsy” refers to the process of removing a tissue sample for diagnostic or prognostic evaluation, and to the tissue specimen itself. Any biopsy technique known in the art can be applied to the methods and compositions of the present invention. The biopsy technique applied will generally depend on the tissue type to be evaluated and the size and type of the tumor (i.e., solid or suspended (i.e., blood or ascites)), among other factors. Representative biopsy techniques include excisional biopsy, incisional biopsy, needle biopsy (e.g., core needle biopsy, fine-needle aspiration biopsy, etc.), surgical biopsy, and bone marrow biopsy. Biopsy techniques are discussed, for example, in Harrison's Principles of Internal Medicine, Kasper, et al., eds., 16th ed., 2005, Chapter 70, and throughout Part V. One skilled in the art will appreciate that biopsy techniques can be performed to identify cancerous and/or precancerous cells in a given tissue sample.

### I. SET_{ER/PR} Index

Embodiments of the present disclosure provide an index of tumoral sensitivity to dose intense chemotherapy, referred to herein as the SET2,3 index. The SET2,3 Index is a measure of the level of transcriptional activity of genes that are related to receptors for the hormones estrogen and progesterone. It combines the SET index of transcription related to estrogen and progesterone receptors (SET_{ER}/P_{R}) with a baseline prognostic index (BPI) derived from pathologic tumor size, nodal involvement, and molecular subtype by RNA4. A “high” SET2,3index was shown to be associated with a good prognosis on endocrine therapies.

The SET_{ER}/P_{R} index is calculated using the expression level of a combination of genes related to both estrogen receptor (ER) and progesterone receptor (PR), such as disclosed in Table 1 including SLC39A6, STC2, CA12, ESR1, PDZK1, NPY1R, CD2, MAPT, QDPR, AZGP1, ABAT, ADCY1, CD3D, NAT1, MRPS30, DNAJC12, SCUBE2, and KCNE4. In some aspects, 5, 6, 7, 8, 9, 10, 11, 12, 13, 14, 15, 16, 17, or 18 of the genes in Table 1 are used to determine the SET_{ER/PR} index. The ER- and PR-related genes can be normalized to reference genes, such as disclosed in Table 1 including LDHA, ATP5J2, VDAC2, DARS, UCP2, UBE2Z, AK2, WIPF2, APPBP2, and TRIM2. In some aspects, 2, 3, 4, 5, 6, 7, 8, 9 or 10 of the reference genes disclosed in Table 1 are used to normalize the expression of the ER- and PR-related genes.

TABLE 1
ESR1- and PGR-associated genes and reference genes.

| Symbol | Name | Entrez ID | Band |
|---|---|---|---|
| SLC39A6 | Solute carrier family 39 (zinc transporter), member 6 | 25800 | 18q12.2 |
| STC2 | Stanniocalcin 2 | 8614 | 5q35.1 |
| CA12 | Carbonic anhydrase XII | 771 | 15q22 |
| ESR1 | Estrogen receptor 1 | 2099 | 6925.1 |
| PDZK1 | PDZ domain containing 1 | 5174 | 1q21 |
| NPY1R | Neuropeptide Y receptor Y1 | 4886 | 4q32.2 |
| CD2 | CD2 molecule | 914 | 1p13.1 |
| MAPT | Microtubule-associated protein tau | 4137 | 17q21.1 |
| QDPR | Quinoid dihydropteridine reductase | 5860 | 4p15.31 |
| AZGP1 | Alpha-2-glycoprotein-1, zinc-binding | 563 | 7q22.1 |
| ABAT | 4-aminobutyrate aminotransferase | 18 | 16p13.2 |
| ADCY1 | Adenylate cyclase 1 | 107 | 7p12.3 |
| CD3D | CD3D molecule, delta (CD3-TCR complex) | 915 | 11q23 |
| NAT1 | N-acetyltransferase 1 (arylamine N-aminotransferase) | 9 | 8p22 |
| MRPS30 | Mitochondrial ribosomal protein S30 | 10884 | 5q11 |
| DNAJC12 | DNAJ (Hsp40) homolog, subfamily C, member 12 | 56521 | 10q22.1 |
| SCUBE2 | Signal peptide, CUB domain, EGF-like 2 | 57758 | 11p15.3 |
| KCNE4 | Potassium channel, voltage-gated subfamily E regulatory subunit 4 | 23704 | 2q36.1 |
| LDHA | Lactate dehydrogenase A | 3939 | 11p15.4 |
| ATP5J2 | ATP synthase, mitochondrial Fo complex, subunit F2 | 9551 | 7q22.1 |
| VDAC2 | Voltage dependent anion channel 2 | 7417 | 10q22 |
| DARS | Aspartylt tRNA synthetase | 1615 | 2q21.3 |
| UGP2 | UDP-glycose phosphorylase 2 | 7360 | 2p14-p13 |
| UBE2Z | Ubiquitin-conjugating enzyme E2Z | 65264 | 17q21.32 |
| AK2 | Adenylate kinase 2 | 204 | 1p34 |
| WIPF2 | WAS/WASL interacting protein family, member 2 | 147179 | 17q21.2 |
| APPBP2 | Amyloid beta precursor protein (cytoplasmic tail) binding protein 2 | 10513 | 17q23.2 |
| TRIM2 | Tripartite motif containing 2 | 23321 | 4q31.3 |

In some aspects, the SET_{ER/PR} index is calculated as:

$${SET}_{ER / PR} = \frac{{\sum }_{i = 1}^{18} {T}_{i}}{i} - \frac{{\sum }_{j = 1}^{10} {R}_{j}}{j} + 2$$

where T, is the expression of the ith of the 18 target genes and R, the expression of the jth of the 10 reference genes. A constant is added to optimize the separation into hormone receptor-positive and negative cases by immunohistochemistry at a score value of 0.

Endocrine Activity Index (EAI) index: Calculation of SET2,3 index (SET_{ER/PR} index adjusted for baseline prognostic index). SET2,3 may be calculated as SET_{ER/PR} index adjusted for a baseline prognostic index (BPI) that includes tumor size, nodal involvement, and RNA4. The pT risk score is calculated from largest pathological tumor dimension and assigned score of zero if ≤10 mm, linearly scaled to the range 0-3.0 if measuring 11-39 mm, assigned risk score 3.0 if ≥40 mm, and assigned risk score 3.0 if the tumor otherwise has stage category pT4.

In particular aspects, the size≤10 mm has score 0, the size≥40 mm has score 3.0 mm. There is a range of 30 mm between those two boundaries (40-10=30), so the score increases by 0.1 unit for every mm it is higher than 10. So, for size between 10 and 40 mm, that score is, size (mm) minus 10, times 0.1. For example, if size is 20 mm, then the score is (20-10)×0.1. The score=1.

The pN risk score may be calculated from the number of involved lymph nodes (0.5 units per node) and assigned score of 5.0 if 10 or more nodes are involved or otherwise stage category pN3. Any lymph node reported as isolated tumor cells (pNOi+) may be considered to be negative for this score.

The RNA4 risk score may be calculated from the expression of ESR1, ERBB2, PGR, and AURKA. Each transcript is normalized by subtracting the mean of the 10 reference genes and adding the constant of 10. Since ESR1, PGR and ERBB2 have bimodal distributions of gene expression in breast cancers, high ESR1 and PGR expression status may be defined as expression exceeding a cut-point two standard deviations (26) below the mean value in the higher expression peak: ESR1=8.93, PGR=5.10. Similarly, the cut-point for ERBB2 gene expression status (11.97) may be defined as 2σ above the mean value of gene expression in the lower expression peak. RNA4 risk scores are calculated as the sum of the risk scores from each gene expression measurement. The RNA4 risk score (range 0-3) depends on the value of AURKA expression in the context of PGR status (Bossuyt et al., Clinical Chemistry, 67:9; 1240-1248, 2021, incorporated by reference herein in its entirety). In some aspects, ff PGR high (>5.1), then AURKA expression levels of 7.0-10.0 were linearly scaled to the range 0-3.0, with AURKA values below 7.0 assigned 0 and values above 10.0 assigned 3.0. If PGR low (≤5.1), then AURKA expression levels of 7.0-8.5 were linearly scaled to the range 0-3.0, with AURKA values below 7.0 assigned 0 and values above 8.5 assigned 3.0. BPI is the sum of scores subtracted from their maximum (so a high score indicates more indolent prognosis) and scaled to the range 0-4.

Specifically, if PGR-high (PGR>5.10), then the AURKA risk score may be calculated as AURKA expression value minus 7.0, but assign 0 if AURKA<7.0, and assign 2.0 if AURKA>9.0 (range of RNA4 risk score 0-2.0).

If PGR-borderline (4.50<PGR≤5.10), add 1.0 to AURKA risk score (range of RNA4 risk score 1.0-3.0).

If PGR-low (PGR≤4.50), then add 1.0 to AURKA risk score, but AURKA risk score is calculated as AURKA expression value minus 7.5, and assigned 0 if AURKA<7.5, and assigned 1.0 if AURKA>8.5 (range of RNA4 risk score 2.0-3.0).

If ESR1-low (ESR1≤8.93), then add 0.5 to the RNA4 risk score.

If ERBB2-high (ESR1≥11.97), then add 0.5 to the RNA4 risk score.

The baseline prognostic index (BPI) may be calculated the sum of risk scores subtracted from 11.0, so: BPI═[11−(pT+pN+RNA4)]×4/11. BPI may be zero-truncated (negative values become zero), so BPI has range from 0 to 4.0, and a higher BPI value represents more indolent baseline prognosis.

SET2,3 index may be calculated as the weighted sum of SET_{ER}/P_{R} index of hormone receptors-related transcriptional activity and the baseline prognostic index (BPI), as follows:

$$SET 2 , 3 = 0.75 * {SET}_{ER / PR} + 0.51 * BPI .$$

### A. Isolation of RNA

Aspects of the present disclosure concern the isolation of RNA from a patient sample for use in determining the SET_{ER}/P_{R} index. The patient sample may blood, saliva, urine, or a tissue biopsy. The tissue biopsy may be a tumor biopsy that has been flash-frozen (e.g. in liquid nitrogen), formalin-fixed and paraffin-embedded (FFPE), and/or preserved by a RNA stabilization agent (e.g., RNAlater). In some aspects, isolation is not necessary, and the assay directly utilizes RNA from within a homogenate of the tissue sample. In certain aspects the homogenate of FFPE tumor sample is enzymatically digested.

RNA may be isolated using techniques well known to those of skill in the art. Methods generally involve lysing the cells with a chaotropic (e.g., guanidinium isothiocyanate) and/or detergent (e.g., N-lauroyl sarcosine) prior to implementing processes for isolating particular populations of RNA. Chromatography is a process often used to separate or isolate nucleic acids from protein or from other nucleic acids. Such methods can involve electrophoresis with a gel matrix, filter columns, coated magnetic beads, alcohol precipitation, and/or other chromatography.

### B. Expression Assessment

In certain aspects, methods of the present disclosure concern measuring expression of ER- and PR-related genes as well as one or more reference genes in a sample from a subject with breast cancer. The expression information may be obtained by testing cancer samples by a lab, a technician, a device, or a clinician. In a certain embodiment, the differential expression of one or more genes including those of Table 5 may be measured.

Expression levels of the genes can be detected using any suitable means known in the art. For example, detection of gene expression can be accomplished by detecting nucleic acid molecules (such as RNA) using nucleic acid amplification methods (such as RT-PCR, droplet-based RT amplification, exon capture of RNA sequence library, next generation RNA sequencing), array analysis (such as microarray analysis), or hybridization methods (such as ribonuclease protection assay, bead-based assays, or Nanostring®. Detection of gene expression can also be accomplished using assays that detect the proteins encoded by the genes, including immunoassays (such as ELISA, Western blot, RIA assay, or protein arrays).

The pattern or signature of expression in each cancer sample may then be used to generate a cancer prognosis or classification, such as predicting cancer survival or recurrence, using the SET_{ER/PR} index. The expression of one or more of ER- and PR-related genes could be assessed to predict or report prognosis or prescribe treatment options for cancer patients, especially breast cancer patients.

The expression of one or more ER- and PR-related genes may be measured by a variety of techniques that are well known in the art. Quantifying the levels of the messenger RNA (mRNA) of a gene may be used to measure the expression of the gene. Alternatively, quantifying the levels of the protein product of ER- and PR-related genes may be to measure the expression of the genes. Additional information regarding the methods discussed below may be found in Ausubel et al., (2003) Current Protocols in Molecular Biology, John Wiley &amp; Sons, New York, NY, or Sambrook et al. (1989) Molecular Cloning: A Laboratory Manual, Cold Spring Harbor Press, Cold Spring Harbor, NY. One skilled in the art will know which parameters may be manipulated to optimize detection of the mRNA or protein of interest.

A nucleic acid microarray may be used to quantify the differential expression of a plurality of ER- and PR-related genes. Microarray analysis may be performed using commercially available equipment, following manufacturer's protocols, such as by using the Affymetrix GeneChip® technology (Santa Clara, CA) or the Microarray System from lncyte (Fremont, CA). Typically, single-stranded nucleic acids (e.g., cDNAs or oligonucleotides) are plated, or arrayed, on a microchip substrate. The arrayed sequences are then hybridized with specific nucleic acid probes from the cells of interest. Fluorescently labeled cDNA probes may be generated through incorporation of fluorescently labeled deoxynucleotides by reverse transcription of RNA extracted from the cells of interest. Alternatively, the RNA may be amplified by in vitro transcription and labeled with a marker, such as biotin. The labeled probes are then hybridized to the immobilized nucleic acids on the microchip under highly stringent conditions. After stringent washing to remove the non-specifically bound probes, the chip is scanned by confocal laser microscopy or by another detection method, such as a CCD camera. The raw fluorescence intensity data in the hybridization files are generally preprocessed with a robust statistical normalization algorithm to generate expression values.

Quantitative real-time PCR (qRT-PCR) may also be used to measure the differential expression of a plurality of ER- and PR-related genes. In qRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified.

For example, extracted RNA can be reverse-transcribed using a GeneAmp® RNA PCR kit (Perkin Elmer, Calif., USA), following the manufacturer's instructions. In some embodiments, gene expression levels can be determined using a gene expression analysis technology that measure mRNA in solution. Methods of detecting gene expression are described for example in U.S. Patent Application Nos. US20140357660, and US20130259858; incorporated herein by reference. Examples of such gene expression analysis technologies include, but not limited to RNAscope™, RT-PCR, Nanostring®, QuantiGene®, gNPA®, HTG®, microarray, and sequencing. For example, methods of Nanostring use labeled reporter molecules, referred to as labeled “nanoreporters,” that are capable of binding individual target molecules. Through the nanoreporters' label codes, the binding of the nanoreporters to target molecules results in the identification of the target molecules. Methods of Nanostring are described in U.S. Pat. No. 7,473,767 (see also, Geiss et al., 2008). Methods may include the RainDance droplet amplification method such as described in U.S. Pat. No. 8,535,889, incorporated herein by reference. Sequencing may include exon capture, such as Illumina targeted sequencing after the generation of a tagged library for next generation sequencing (e.g. described in International Patent Application No. WO2013131962, incorporated herein by reference).

A non-limiting example of a fluorescent reporter probe is a TaqMan® probe (Applied Biosystems, Foster City, CA). The fluorescent reporter probe fluoresces when the quencher is removed during the PCR extension cycle. Multiplex qRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, qRT-PCR is typically performed using a reference standard. The ideal reference standard is expressed at a constant level among different tissues, and is unaffected by the experimental treatment. The system can include a thermocycler, laser, charge-coupled device (CCD) camera, and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

To minimize errors and the effect of sample-to-sample variation, RT-PCR can be performed using an internal standard. The ideal internal standard is expressed at a constant level among different tissues, and is unaffected by an experimental treatment. RNAs commonly used to normalize patterns of gene expression are mRNAs for the housekeeping genes GAPDH, β-actin, and 18S ribosomal RNA.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Heid et al., 1996). Quantitative PCR is also described in U.S. Pat. No. 5,538,848. Related probes and quantitative amplification procedures are described in U.S. Pat. Nos. 5,716,784 and 5,723,591. Instruments for carrying out quantitative PCR in microtiter plates are available from PE Applied Biosystems (Foster City, CA).

The steps of a representative protocol for quantitating gene expression level using fixed, paraffin-embedded tissues as the RNA source, including mRNA isolation, purification, primer extension and amplification are given in various published journal articles (see Godfrey et al., 2000; Specht et al., 2001). Briefly, a representative process starts with cutting about 10 μιη thick sections of paraffin-embedded neoplasm tissue samples or adjacent non-cancerous tissue. The RNA is then extracted, and protein and DNA are removed. Alternatively, RNA is isolated directly from a neoplasm sample or other tissue sample. After analysis of the RNA concentration, RNA repair and/or amplification steps can be included, if necessary, and RNA is reverse transcribed using gene specific primers, followed by preparation of a tagged RNA sequencing library, and paired-end sequencing. In another example, the RNA is not reverse transcribed, but is directly hybridized to a specific template and then labeled with oligonucleotides and/or chemical or fluorescent color to be detected and counted by a laser.

Immunohistochemical staining may also be used to measure the differential expression of a plurality of ER- and PR-related genes. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of a plurality of ER- and PR-related genes. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of a plurality of ER- and PR-related genes. For this, a plurality of antibodies is arrayed and covalently attached to the surface of the microarray or biochip. A protein extract containing the biomarker proteins of interest is generally labeled with a fluorescent dye.

The labeled ER- and PR-related genes proteins may be incubated with the antibody microarray. After washes to remove the unbound proteins, the microarray is scanned. The raw fluorescent intensity data may be converted into expression values using means known in the art.

Luminex multiplexing microspheres may also be used to measure the differential expression of a plurality of biomarkers. These microscopic polystyrene beads are internally color-coded with fluorescent dyes, such that each bead has a unique spectral signature (of which there are up to 100). Beads with the same signature are tagged with a specific oligonucleotide or specific antibody that will bind the target of interest (i.e., biomarker mRNA or protein, respectively). The target, in turn, is also tagged with a fluorescent reporter. Hence, there are two sources of color, one from the bead and the other from the reporter molecule on the target. The beads are then incubated with the sample containing the targets, of which up 100 may be detected in one well. The small size/surface area of the beads and the three dimensional exposure of the beads to the targets allows for nearly solution-phase kinetics during the binding reaction. The captured targets are detected by high-tech fluidics based upon flow cytometry in which lasers excite the internal dyes that identify each bead and also any reporter dye captured during the assay. The data from the acquisition files may be converted into expression values using means known in the art.

In situ hybridization may also be used to measure the differential expression of a plurality of biomarkers. This method permits the localization of mRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an mRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple mRNAs may be detected simultaneously, provided each antisense probe has a distinguishable label. The hybridized tissue array is generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for each biomarker.

### C. ESR1 Mutations

Activating mutations in the estrogen receptor gene, ESR1, are a key mechanism in acquired endocrine resistance in breast cancer therapy. Accordingly, some aspects of the present invention further refine the SET_{ER/PR} index by including variables for the expression of mutated ESR1. The presence of transcript expressing a mutated form of ESR1 is detected by specific primers that amplify a specific part of the ligand-binding domain sequence of ESR1 transcript that is known to be a region that is enriched for activating mutations. The proportion of the transcript expressing a mutated form of ESR1 is calculated as the expression of mutated ESR1 over the expression of ESR1 measured using different primers that detect a region of the ESR1 transcript that is reliably expressed in samples and is not prone to mutation. In one example, the mutation status is incorporated logistically with SET index status (yes/no combined with high/low). In another example, the mutation status of the transcript, the proportion of ESR1 transcript that is mutated, and the SET index value are incorporated into a multivariable index score, where the coefficients of the score are based on multivariable Cox regression model of prognosis following endocrine therapy.

Mutations of ESR1 are known in the art. For example, five ESR¹ mutations identified encoding p.Leu536Gln, p.Tyr537Ser, p.Tyr537Cys, p.Tyr537Asn and p.Asp538Gly were shown to result in constitutive activity and continued responsiveness to anti-estrogen therapies in vitro (Robinson et al., 2013). Other ESR1 mutations include S463P, V534E, P535H, L536Q, L536R, Y537C, Y537S, Y537N, and D538G.

### II. Methods of Treatment

Provided herein are methods for treating or delaying progression of breast cancer in an individual determined to be sensitive to dose intense chemotherapy using the SET_{ER}/P_{R} or SET2/3 index comprising administering to the individual an effective amount of a dose intense chemotherapy. The breast cancer may be Stage II, Stage 111, or Stage IV breast cancer and, in particular aspects, the Stage IV breast cancer is metastatic and relapsed after prior treatments. In certain aspects, the breast cancer is hormone receptor-positive (i.e., positive for the receptors for the hormones estrogen (ER-positive cancers) and/or progesterone (PR-positive cancers) and/or HER2-negative.

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocannycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carnustine, chlorozotocin, fotemustine, lomustine, nimustine, and ranimnustine; antibiotics, such as the enediyne antibiotics (e.g., calicheamicin, especially calicheamicin gammalI and calicheamicin omegal1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

In some aspects, dose intense (also referred to herein as dose intense) chemotherapy may comprise a 2-weekly chemotherapy as outlined below. Dose-intense chemotherapy may comprise an anthracycline (e.g., doxorubicin or epirubicin) and/or taxane (e.g., paclitaxel or docetaxel) on a 2/week schedule. For example, dose intense chemotherapy may comprise paclitaxel at a dose-intense 2-weekly regimen with hematological colony stimulating factor to support the bone marrow (e.g. ddAC/T).

TABLE 2
Summary of Contemporary Adjuvant Chemotherapy Regimens for Breast Cancer.

| | Anthracycline | | Anthracycline-Taxane | | Taxane Only | |
|---|---|---|---|---|---|---|
| Dosing Schedule | Doxorubicin | Epirubicin | Paclitaxel | Docetaxel | Paclitaxel | Docetaxel |
| Conventional | AC, FAC, | FEC, EC | T/FAC, | FEC/D, TAC, | — | “TC” |
| q3wk | CAF | | AC/T (“P3”) | AC/D (“D3”) | | |
| Dose-intense | ddAC | ddEC | ddAC/T | ddEC/T | ddT | — |
| q2wk, +GM- | | | | | | |
| CSF | | | | | | |
| Weekly | — | — | FEC/wT, | AC/wD | WT | — |
| q1wk, lower dose | | | wT/FAC, | (“D1”) | | |
| | | | AC/wT (“P1”) | | | |

Abbreviations: A, doxorubicin; E, epirubicin; C, cyclophosphamide; F, fluorouracil; T, paclitaxel; D, docetaxel; q3wk, every 3 weeks; dd, dose-intense q2-weekly with GM-CSF colony stimulating factors for bone marrow support; w, weekly; P3, D3, P1, and D1: 4 treatment arms from ECOG E-1199 trial.

In some aspects, a subject with a low SET_{ER/PR} or SET2,3 may be administered dose-intense chemotherapy, such as paclitaxel, comprising weekly or high-dose 2-weekly schedule. In certain aspects, a subject with a high SET_{ER/PR} or SET2,3 may be administered conventional chemotherapy on a 3-weekly schedule. Exemplary dose-intense chemotherapy may comprise sequential administration of 3 cycles of epirubicin (150 mg/m²) and cyclophosphamide (e.g., 600 mg/m² per treatment for a total 2,400 mg/m²), and paclitaxel (225 mg/m²), at 2-week intervals (q2w) with epoetin alfa and filgrastim support. An exemplary dose-intense chemotherapy may instead administer the paclitaxel at weekly intervals (q1w) at lower dose (80 mg/m²). An exemplary convention chemotherapy may comprise 4 cycles of epirubicin/cyclophosphamide (90/600 mg/m²) followed by 4 cycles of paclitaxel (175 mg/m²) q3w.

In some embodiments, the individual has cancer that is resistant (has been demonstrated to be resistant) to one or more anti-cancer therapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage.

In some aspects, the patient has been previously administered a hormonal therapy and/or additional anti-cancer therapy. For example, the patient may have been administered a hormonal therapy in combination with chemotherapy, such as for five years. In some aspects, the patients has shown previous sensitivity to a hormonal therapy.

In some aspects, the dose intense chemotherapy is administered in combination with at least one additional anti-cancer therapy, such as hormonal therapy or immunotherapy. The chemotherapy may be administered before, during, after, or in various combinations relative to the additional anti-cancer agent. The administrations may be in intervals ranging from concurrently to minutes to days to weeks. In embodiments where the chemotherapy is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the chemotherapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Exemplary hormonal therapies for breast cancer include the SERM, AI, and SERD classes of drugs that inhibit the activity of the estrogen and estrogen-receptor complex, such as tamoxifen, toremifene, and fulvestrant. Other hormonal therapies include treatments to lower estrogen levels including aromatase inhibitors such as letrozole, anastrozole, and exemestane. Permanent ovarian ablation can be done by surgically removing the ovaries. This operation is called an oophorectomy. More often, ovarian ablation is done with drugs called luteinizing hormone-releasing hormone (LHRH) analogs, such as goserelin (Zoladex®) or leuprolide (Lupron®). These drugs stop the signal that the body sends to ovaries to make estrogens. They can be used alone or with other hormone drugs (tamoxifen, aromatase inhibitors, fulvestrant) as hormone therapy in pre-menopausal women. The effectiveness of hormonal therapy may also be enhanced by the addition of an additional therapy to synergistically inhibit a different biological pathway, such as palbociclib (Cdk4/6 inhibitor), everolimus (mTOR/PI3K inhibitor), immune therapy, or other therapies.

The chemotherapy and, optionally the anti-cancer agent, may be administered by the same route of administration or by different routes of administration. In some embodiments, the chemotherapy and/or anti-cancer agent is administered intravenously, intramuscularly, subcutaneously, topically, orally, transdermally, intraperitoneally, intraorbitally, by implantation, by inhalation, intrathecally, intraventricularly, or intranasally. An effective amount of the chemotherapy and/or anti-cancer agent may be administered for prevention or treatment of disease. The appropriate dosage of the chemotherapy and anti-cancer agent be determined based on the type of disease to be treated, severity and course of the disease, the clinical condition of the individual, the individual's clinical history and response to the treatment, and the discretion of the attending physician.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

### A. Pharmaceutical Compositions

Also provided herein are pharmaceutical compositions and formulations comprising the dose intense chemotherapy, optionally an anti-cancer agent and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22nd edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX©, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### B. Anti-Cancer Therapy

In certain embodiments, the compositions and methods of the present embodiments involve dose intense chemotherapy in sequence or combination with at least additional anti-cancer agent. The additional therapy may be radiation therapy, surgery (e.g., lumpectomy and a mastectomy), chemotherapy, targeted molecular inhibitor, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant, neoadjuvant, or palliative therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting receptor or receptor kinase signaling molecules, cyclin-dependent kinases or the cell cycle control, mTOR/PI3K pathway, HSP90 inhibitor, tubulin inhibitor, apoptosis inhibitor, and/or chemopreventative agent. The additional therapy may be one or more of the chemotherapeutic agents known in the art.

Various combinations may be employed. For the example below a dose intense chemotherapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Radiotherapy

Other factors that cause DNA damage and have been used extensively include what are commonly known as 7-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 2. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen (Carter et al., 2008; Teicher 2014; Leal et al., 2014). Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach (Teicher 2009) and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fe region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA*, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; Camacho et al., 2004; Mokyr et al., 1998 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as IOD1, MDX-010, MDX-101, and Yervoy®) or antigen binding fragments and variants thereof (see, e.g., WOO 1/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesions such as described in U.S. Pat. No. 8,329,867, incorporated herein by reference.

### 3. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 4. Other Agents

It is contemplated that other agents may be used in combination with certain aspects of the present embodiments to improve the therapeutic efficacy of treatment. Recently validated and approved clinical examples include the concurrent administration of hormonal therapy with a biotherapy that inhibits the cell cycle (e.g., palbociclib) or the mTOR/PT3K pathway (e.g., everolimus). Further examples can therefore be contemplated. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### III. Articles of Manufacture or Kits

Further embodiments of the invention include kits for the measurement, analysis, and reporting of ER- and PR-related gene expression and transcriptional output. A kit may include, but is not limited to microarray, quantitative RT-PCR, or other genomic platform reagents and materials, as well as hardware and/or software for performing at least a portion of the methods described. For example, custom microarrays or analysis methods for existing microarrays are contemplated. Accordingly, an article of manufacture or a kit is provided comprising a customized assay for determining the SET2,3 index also provided herein. The article of manufacture or kit can further comprise a package insert comprising instructions for using the customized assay to determine the SET2,3 index and to then treat or delay progression of breast cancer in an individual. Probes for any of the ER- and PR-related genes described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. Suitable containers for the one or more agent include, for example, bottles, vials, bags and syringes.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Measurement of Endocrine Activity Index (EAI) Related to Prognosis and Prediction of Benefit from Dose-Intense (DD) Chemotherapy in Estrogen Receptor-Positive (ER+) Cancer

### C9741 Study Population

The study enrolled women diagnosed with primary node-positive adenocarcinoma of the breast (pathologic stage T0 to T3, N1/2, MO) treated with upfront breast surgery.¹ Eligibility criteria were previously reported.¹Estrogen receptor (ER) status was reported form local testing, but HER2 status was not routinely assessed during the conduct of the study. Total RNA was previously extracted from all available primary tumor blocks from C9741 to measure the ROR-PT score and determine intrinsic subtype, as previously described by Liu et al.⁶ Applicants study cohort for evaluation of Endocrine Activity Index (EAI) assay was limited to subjects with ER-positive breast cancer and at least 200 ng of residual RNA remaining.⁶

### RNA Samples and Endocrine Activity Index (EAI) Assay

Total RNA had been purified from the primary tumor blocks from 1311 patients who were treated on C9741 in 2011 for genomic testing with the research version of the Prosigna® hybridization assay (Nanostring Technologies®, Seattle, WA) in the Clinical Pathology Department at Washington University St. Louis and residual RNA had been returned to the biorepository (The Alliance for Clinical Trials) for storage at −80° C.⁶ Applicants obtained permission from the Cancer Correlative Sciences Committee (protocol CCSC-0154) for the National Clinical Trials Network (NCTN) of the National Cancer Institute (NCI) to receive a 300 ng aliquot (or otherwise at least 200 ng) of this stored RNA from each of 682 eligible samples from the 822 patients in this cohort who had estrogen receptor-positive (ER+) breast cancer by local determination. The ROR-PT score, pre-defined cut point (≤50, >50), and intrinsic subtype assignments were used for this study as previously published.⁶

The published 31-gene SET2,3 Index (also referred to as Endocrine Activity Index (EAI), see Table 1) was measured from total RNA using the QuantiGene Plex (QGP) platform (Thermo Fisher Scientific, Waltham, MA) and was performed as per manufacturer's protocol.^{18,20} Briefly, the QGP assay involves hybridization of target RNA to the oligonucleotide probes that coat specific beads, followed by signal amplification with secondary oligonucleotides and labeling with streptavidin phycoerythrin (SAPE).²¹ The Luminex 200 instrument (Luminex, Austin, TX) counts the SAPE signals for each bead, specific to each probe.²¹ Results passed quality control for analysis if the mean of the 10 reference genes was >3.5 (log 2 counts).¹⁸ SET2,3 index and its components, SET_{ER/PR} and BPI, were calculated directly from the log 2 transformed counts from QGP gene measurements, tumor size and number of involved lymph nodes, as previously reported.¹⁵ Results of the Endocrine Activity Index (EAI) assay were provided to the C9741 trial statistician (KB) for independent analysis.

### Study Endpoints

C9741 primary endpoint was defined as disease-free survival (local recurrence, distant relapse, or death without relapse) measured from the time of study entry.¹ Isolated contralateral breast recurrence, and second primary malignancies were classified as adverse events and not failure in DFS. The secondary end point overall survival was measured from study entry until death from any cause.¹ Death as a result of acute myelogenous leukemia (AML)/myelodysplastic syndrome (MDS) was considered treatment related.¹

### Statistical Methods

C9741 evaluated the effects of adjuvant chemotherapy dose density (2 weekly v 3 weekly), and treatment sequence (concurrent v sequential), and their possible interaction. It was designed to detect a 33% difference in hazard for either main effect expecting a total of 515 events (i.e., relapses or death), with 90% power at significant level of 5% with a target enrollment of 1,584 patients over 22 months, and 3-years of follow-up after the last patient accrued. A total of 2005 patients were enrolled between September of 1997 and March of 1999, and sample size was increased to correct for the faster than anticipated accrual rate.¹ The initial analyses were performed with a median follow-up of 36 months, and a total of 315 events (i.e., disease relapse or death). The current analyses describe the final DFS and OS analyses including multiple events with 12.3 years of median follow-up. Survival curves were generated for each group using a Kaplan-Meier estimator and the curves compared with a log rank test (prespecified level of significance was p<0.05).

Endocrine Activity Index (EAI) was evaluated as a continuous index (per unit) and categorically using the predefined cut point (SET2,3≥2.10 is high, SET2,3<2.10 is low) and adjusted for treatment arm.^{15,19} The protocol predefined three objectives. The first was to determine whether high EAI was associated with favorable disease-free survival (DFS), defined as 5-year DFS greater than 78% (i.e., 95% confidence interval entirely above 78%). The second objective was to compare the EAI with the risk of recurrence score that is combined with proliferation index and tumor size (ROP-PT score) and the intrinsic subtype of each sample⁶ Applicants evaluated their correlation as continuous variables (Spearman rank correlation) and agreement by category. Applicants used multivariable Cox regression models, adjusting for treatment arm, to compare their prognostic performance as continuous indices and as categories using the pre-defined cut points. The third objective was to determine whether efficacy of chemotherapy dosing schedules in ER-positive cancer depends on EAI, or secondly whether it depends on ROR-PT score. A Cox model was evaluated that included EAI (continuous variable), chemotherapy intensity (dose-intense versus conventional), and their interaction term with prespecified level of significance for interaction term was p<0.1). If an interaction was identified, then it was explored for an effect from menopausal status (based on the results of the RxPonder trial) or from HER2 status (because HER2 testing was not reported during the C9741 trial). HER2-positive status was inferred from HER2-enriched intrinsic subtype in the Prosigna data, or alternatively, from ERBB2 gene expression level above the pre-defined cut point from the Endocrine Activity Index (EAI) results.

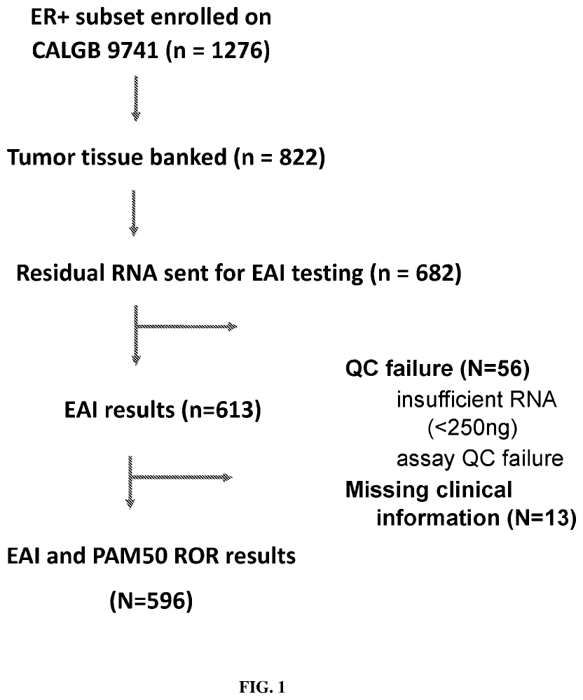

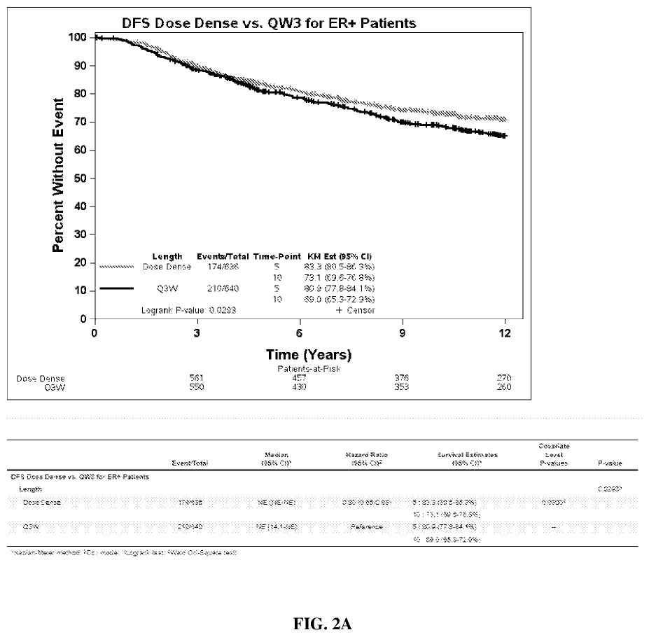

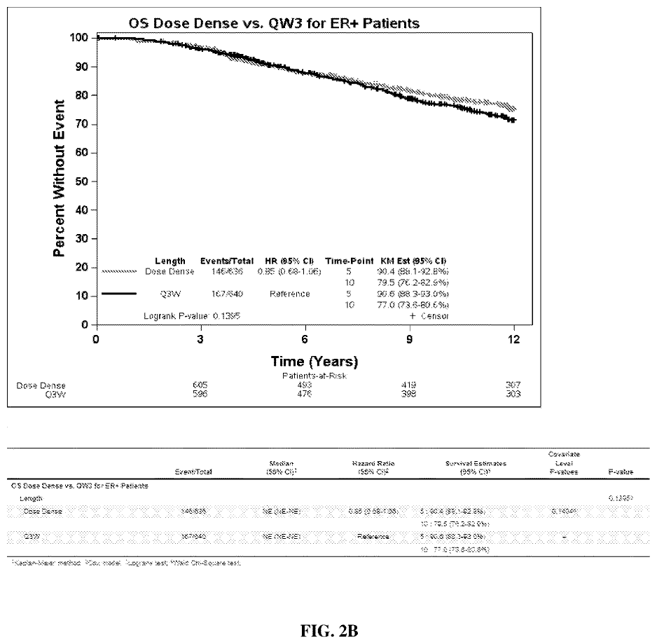

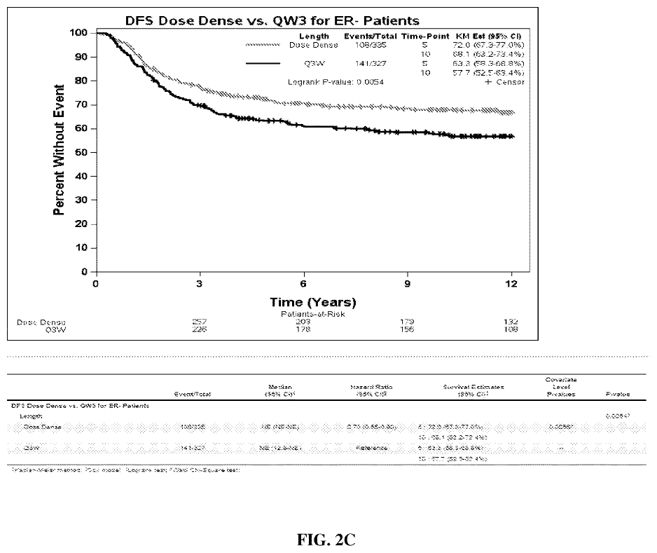

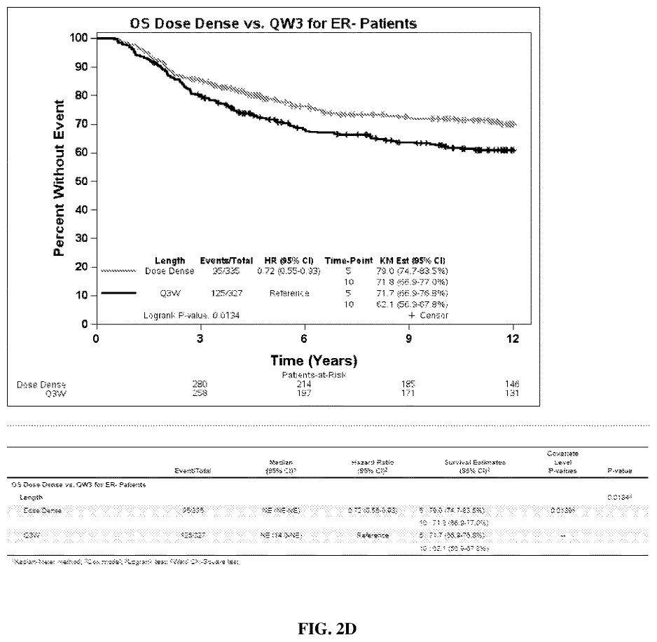

The C9471 efficacy population included a total of 1,973 patients (FIG. 1). As previously reported, 65% of the study population had estrogen receptor positive tumors, and the median number of involved axillary nodes was three. At 12.3 years of median follow-up in patients with ER-positive cancer, DFS was significantly longer for dose-intense v. q3w regimens (HR=0.80, 95% CI 0.65 to 0.98) (FIG. 2A). The estimated DFS rates for the dose intense and conventional q3w schedules were 83.3% (95% CI 80.5 to 86.3) and 80.9% (95% CI 77.8 to 84.1) at year 5. The treatment effect remained statistically significant when adjusting for number of positive nodes, tumor size, and menopausal status. The overall relative reduction in hazard of death for patients with ER-positive cancer is depicted in FIG. 2B. The overall relative reduction in hazard of recurrence for ER-negative tumors is depicted in FIG. 2C. The overall relative reduction in hazard of death for patients with ER-negative cancer is depicted in FIG. 2D. Concurrent vs sequential regimen was not correlated with DFS or OS, nor there was evidence of an interaction between dose density and treatment sequence for DFS and OS.

The acute toxicity data associated with adjuvant chemotherapy along with dose intensity and drug discontinuation have been reported previously.¹At 12 years of median follow-up there were treatment related deaths including of doxorubicin induced cardiomyopathy. Certain cases of MDS and certain cases of AML which were evenly distrusted across study arms. See Table 3 below illustrating Treatment related deaths.

TABLE 3
Treatment related deaths.

| | Survival | |
|---|---|---|
| Regimen | (months) | Cause of Death |
| I | 30 | Heart failure |
| I | 40 | AML |
| I | 41 | AML |
| II | 23 | AML |
| III | 30 | MDS |
| III | 39 | Infection secondary to AML |

NOTE.
Regimen I, sequential doxorubicin → paclitaxel → cyclophosphamide every 3 weeks; regimen II, sequential doxorubicin → paclitaxel → cyclophosphamide every 2 weeks; regimen III, concurrent doxorubicin and cyclophosphamide every 3 weeks followed by paclitaxel every 3 weeks; regimen IV, concurrent doxorubicin and cyclophosphamide every 2 weeks followed by paclitaxel every 2 weeks (see text for details).
Abbreviations:
AML, acute myelogenous leukemia;
MDS, myelodysplastic syndrome.

The incidence of AML or MDS is shown above, and similar to the 0.18% incidence reported at 3-years of median follow-up. See Table 4 below. The incidence of AML/MDS was not different across study arms.

TABLE 4
Incidence of AML or MDS.

| | I | II | III | IV |
|---|---|---|---|---|
| | (no. of | (no. of | (no. of | (no. of |
| | patients) | patients) | patients) | patients) |
| Total treated | 484 (100%) | 493 (100%) | 501 (100%) | 495 (100%) |
| Total with second primary | 16 (3%) | 16 (3%) | 12 (2%) | 14 (3%) |
| Contralateral breast | 9 | 2 | 6 | 1 |
| DCIS | 1 | 1 | 0 | 1 |
| Cervix | 1 | 0 | 0 | 1 |
| Ovary | 0 | 1 | 0 | 0 |
| Endometrium | 0 | 1 | 0 | 1 |
| AML/MDS | 2 | 3 | 4 | 2 |
| Basal/squamous | 0 | 3 | 1 | 2 |
| Melanoma | 1 | 1 | 0 | 1 |
| Lung | 0 | 2 | 1 | 0 |
| Thyroid | 0 | 0 | 0 | 2 |
| Colon | 0 | 0 | 0 | 1 |
| Intestine | 0 | 0 | 0 | 1 |
| Bladder | 0 | 0 | 0 | 1 |
| Renal | 2 | 0 | 0 | 0 |
| Pancreas | 0 | 1 | 0 | 0 |
| Pituitary | 0 | 1 | 0 | 0 |

NOTE.
Regimen I, sequential doxorubicin → paclitaxel → cyclophosphamide every 3 weeks; regimen II, sequential doxorubicin → paclitaxel → cyclophosphamide every 2 weeks; regimen III, concurrent doxorubicin and cyclophosphamide every 3 weeks followed by paclitaxel every 3 weeks; regimen IV, concurrent doxorubicin and cyclophosphamide every 2 weeks followed by pacilitaxel every 2 weeks (see text for details).
Abbreviations:
DCIS, ductal carcinoma-in-situ;
AML, acute myelogenous leukemia;
MDS, myelodysplastic syndrome.

Prognosis According to EAI Index and its Combination with ROR-PT Score

Of the 1276 pts with ER+ breast cancer included in the study, 822 had formalin-fixed paraffin embedded (FFPE) banked, with residual RNA available for 682 participants (FIG. 1). Of these, Endocrine Activity Index (EAI) was successfully obtained for 613 participants (56 RNA quality control failure and 13 patients with missing clinico-pathologic information). When compared to the ER+ population, the subset with EAI results had similar clinico-pathologic characteristics expect for higher tumor size (>2 cm, 61% vs 50%) and higher rates of mastectomy (67% vs. 57%), See Table 4 above.

Endocrine Activity Index (EAI) was prognostic for DFS (HR 0.47, 95% CI 0.35-0.64, p<0.001) and OS (HR 0.38, 95% CI 0.27-0.54, p<0.001). At 5 years, patients with high EAI had DFS of 0.86 (95% CI 0.81-0.90) that was significantly higher than the pre-specified threshold of 0.78, and higher than the observed 5-year DFS of 0.73 for patients with low EAI. At 10 years, the observed DFS was 0.78 and 0.52 for high and low EAI, respectively. Similarly, for overall survival, 5-year and 10-year OS of 0.95 and 0.87 were observed, respectively, in patients with high EAI; and 5-year and 10-year OS of 0.85 and 0.66, respectively, in patients with low EAI. The superior DFS and OS outcomes for high v. low EAI were maintained when evaluating EAI as a continuous variable.

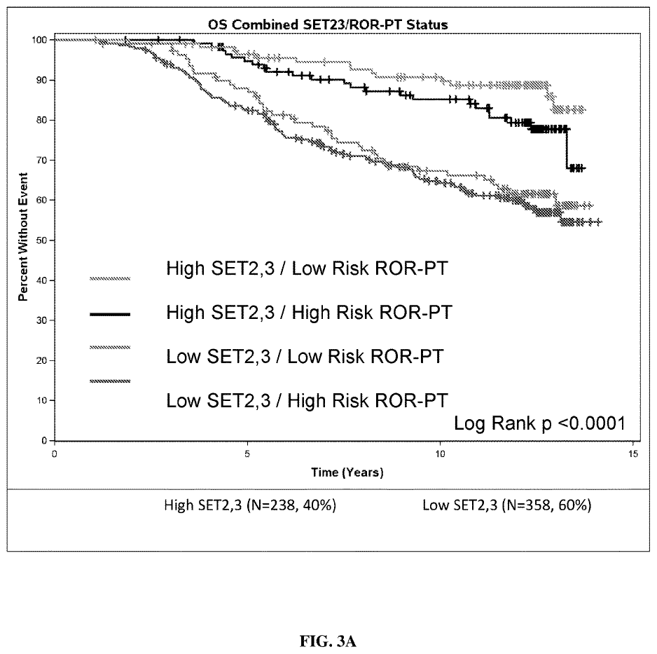

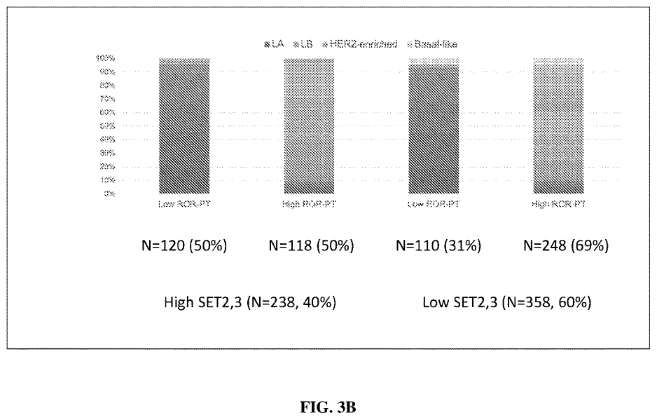

ROR-PT and intrinsic subtype results were available for 596 of the 613 tumors with EAI results. Endocrine Activity Index (EAI) and ROR-PT score had minimal correlation (Spearman correlation coefficient=0.31; 95% CI, −0.38 to −0.23). High EAI index was prognostic (HR 0.46, 95% Cl 0.34-0.63, p<0.001) but high ROR-PT score was not independently prognostic (HR 1.22, 95% CI 0.91-1.64) in a multivariable Cox model for DFS that was adjusted for treatment arm. Kaplan Meier plots demonstrated that ROR-PT status added little to the prognostic assessment by EAT status for DFS and OS (FIG. 3A). The distribution of low- and high-SET2,3 tumors according to PAM50 intrinsic subtypes, ROR-PT risk, and its prognostic associations is summarized in FIG. 3B. High EAI index was observed in 52% (120/230) of cancers with low-risk ROR-PT score and 32% (118/366) of cancers with high-risk ROR-PT score. Using PAM50 intrinsic subtypes, high EAI index was observed in 49% (123/249) of luminal A, 47% (113/240) of luminal B, 2% (2/87) of HER2-enriched, and none of 20 basal-like cancers.

Table 5 below shows a tabulation of results of the prognostic performance of ROR-PT multivariate model for Disease Free Survival DFS (categorical ROR-PT; high ROR-PT>50).

| | Hazard | 95% confidence | p- |
|---|---|---|---|
| Variable | Ratio | interval | value |
| ROR-PT | | | 0.023 |
| low | 1.00 | reference | |
| high | 1.40 | 1.05 to 1.86 | |
| Treatment arm | | | |
| I: A-C-T, every 3 weeks | 1.00 | reference | 0.44 |
| II: A-C-T, every 2 weeks | 0.81 | 0.56 to 1.19 | |
| III: AC-T, every 3 weeks | 0.99 | 0.68 to 1.44 | |
| IV: dose intense AC-T | 0.78 | 0.53 to 1.14 | |

Table 6 below summarizes prognostic performance of Set2,3 and ROR-PT multivariate Cox Model for Disease Free Survival (DFS).

| | Hazard | 95% confidence | |
|---|---|---|---|
| Variable | Ratio | interval | p-value |
| SET2,3 | | | <0.0001 |
| low | 1.00 | reference | |
| high | 0.46 | 0.34 to 0.63 | |
| ROR-PT | | | 0.18 |
| low | 1.00 | reference | |
| high | 1.22 | 0.91 to 1.64 | |
| Treatment arm | | | |
| I: A-C-T, every 3 weeks | 1.00 | reference | 0.28 |
| II: A-C-T, every 2 weeks | 0.87 | 0.59 to 1.27 | |
| III: AC-T, every 3 weeks | 1.01 | 0.69 to 1.45 | |
| IV: dose intense AC-T | 0.71 | 0.49o 1.05 | |

Prediction of Benefit from Dose-Intense Chemotherapy

Evaluating DFS outcomes, EAT (p<0.0001) and dose density (p=0.021) were statistically significant, with significant predictive interaction (P_{interaction}=0.09) at the pre-specified 0.10 level. Similarly, when evaluating OS outcomes, SET2,3 (P<0.0001), dose density (P=0.009) and their interaction term (P_{interaction}=0.027 for interaction) were statistically significant. The predictive association between EAI and dose-intense benefit were apparently more pronounced with lower EAI levels (e.g., 33^{rd} lower percentile) for both DFS and OS as opposed to higher EAI levels (e.g., 33^{rd} upper percentile).

Menopausal status did not predict benefit from dose-intense chemotherapy in terms of DFS (P_{interaction}=0.44), RFS (P_{interaction}=0.37) or OS (P_{interaction}=0.30), although menopausal status was independently prognostic in these Cox models of DFS (P=0.045), RFS (P=0.047) and OS (P=0.016). Subset analyses demonstrated that prognosis for postmenopausal women was significantly different from premenopausal women after conventional chemotherapy in terms of DFS (HR 1.47, 95% CI 1.01-2.14), RFS (HR 1.50, 95% CI 1.10-2.23) and OS (HR 1.70, 95% CI 1.11-2.61); but was not significantly different after dose-intense chemotherapy. Note that low EAI was more common in postmenopausal women than in premenopausal women [211/332 (64%), versus 154/277 (56%), P=0.046].

ROR-PT score did not predict benefit from dose-intense chemotherapy in terms of DFS (P_{interaction}=0.37), RFS (P_{interaction}=0.32) or OS (P_{interaction}=0.37), although ROR-PT score was independently prognostic in these Cox models of DFS (P=0.022), RFS (P=0.001) and OS (P=0.002). Finally, there was no predictive interaction between the sequence of anthracycline and cyclophosphamide treatments (concurrent versus sequential treatment arms) and EAI, menopausal status, or ROR-PT score.

### Example 2: Efficacy of Dose-Intense Chemotherapy by SET2,3 (Endocrine Activity Index (EAI)) Results

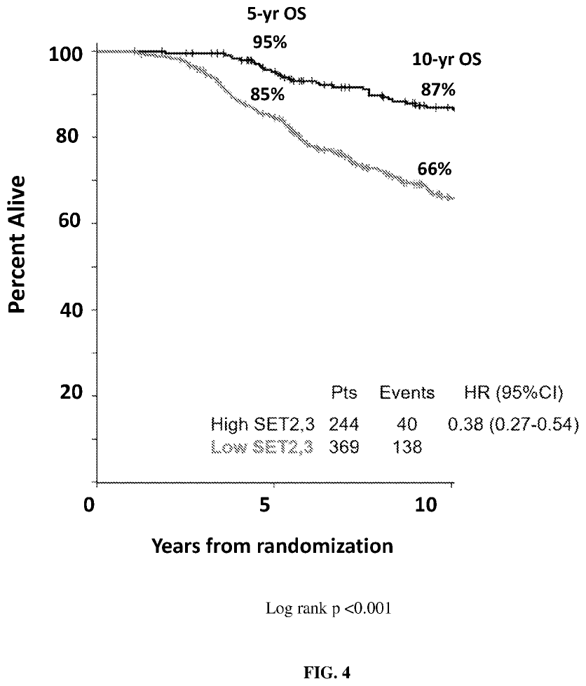

Endocrine Activity Index (EAI) results inform the benefit from dose-intense versus conventional chemotherapy in ER+ breast cancer. FIG. 4 illustrates the overall survival of subjects with low Set2,3 and high Set2,3 and frames a baseline understanding of the prognostic performance of the SET2,3 (Endocrine Activity Index (EAI) test.

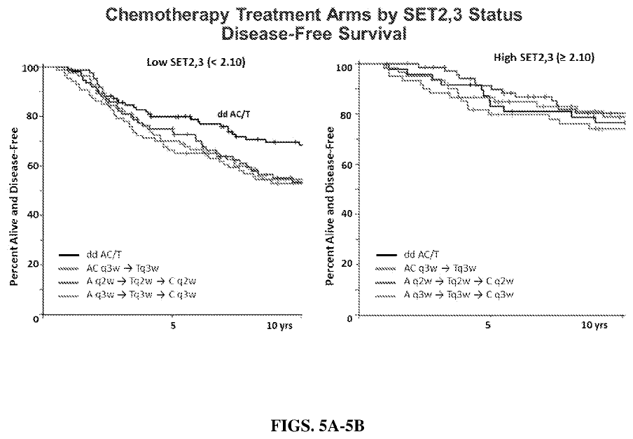

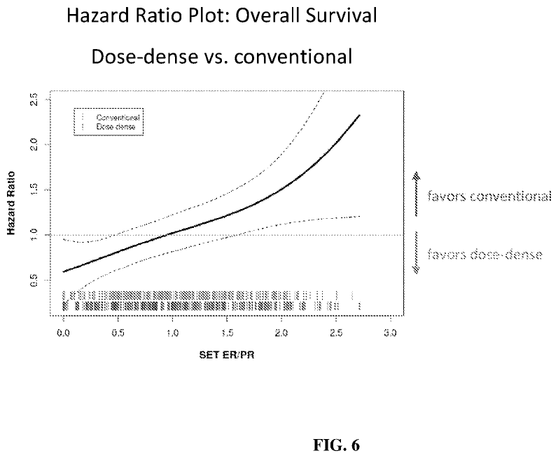

FIG. 5A illustrates that subject with Low Set2,3 (<2.10) have superior DFS outcomes with dose intense chemotherapy. For subjects with high Set2,3 (≥2.10) the outcomes of dose intense chemotherapy and standard chemotherapy regimens tend to be similar (FIG. 5B).

Table 7 below provides a tabulation of the results for the efficacy of dose-intense Chemotherapy by SET2,3 Index Disease-Free Survival.

| | P- | HR (95% CI) for SET2, 3 | | HR (95% CI) for ChemoRx | | |
|---|---|---|---|---|---|---|
| | value | index | | (conventional versus dose-intense) | | |
| | 2- | | Dose- | SET2, 3 = | SET2, 3 = | SET2, 3 = |
| Term | sided | Conventional | intense | 1.0 | 2.0 | 3.0 |
| SET2, 3 | <0.001 | 0.44 (0.33, | 0.61 (0.47, | | | |
| (continuous) | | 0.59) | 0.79) | | | |
| ChemoRx | 0.021 | | | 1.61 (1.12, | 1.17 (0.86, | 0.65 (0.47, |
| | | | | 2.30) | 1.58) | 1.52) |
| Interaction | 0.098* | | | Lower Set2, 3 value favors Dose Intense | | |
| Term ** | | | | | | |

Table 8. Efficacy of Dose-Intense Chemotherapy by SET2,3 Index Overall Survival.

| | P- | HR (95% CI) for SET2, 3 | | HR (95% CI) for ChemoRx | | |
|---|---|---|---|---|---|---|
| | value | index | | (conventional versus dose-intense) | | |
| | 2- | | Dose- | SET2, 3 = | SET2, 3 = | SET2, 3 = |
| Term | sided | Conventional | intense | 1.0 | 2.0 | 3.0 |
| SET2, 3 | <0.001 | 0.38 (0.28, | 0.60 (0.46, | | | |
| (continuous) | | 0.51) | 0.80) | | | |
| ChemoRx | 0.0096 | | | 1.63 (1.11, | 1.01 (0.71, | 0.63 (0.32, |
| | | | | 2.38) | 1.42) | 1.22) |
| Interaction | 0.027 | | | | | |
| Term | | | | | | |

### Example 3: Set_{ER/PR} Index

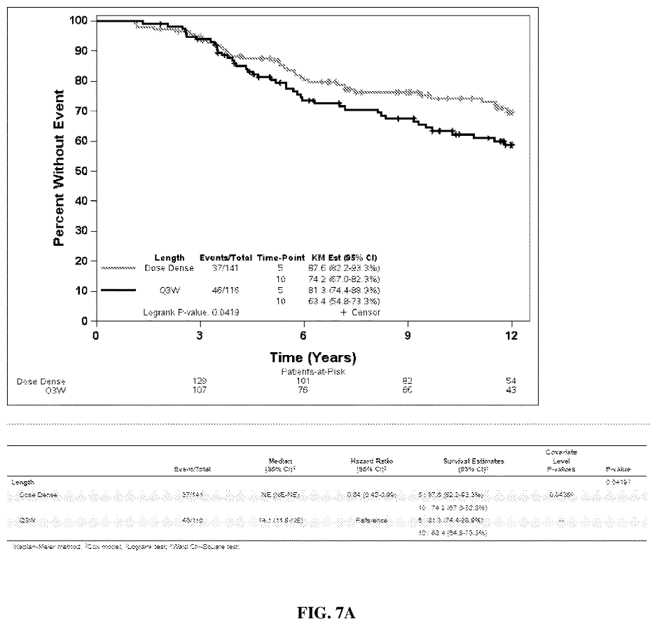

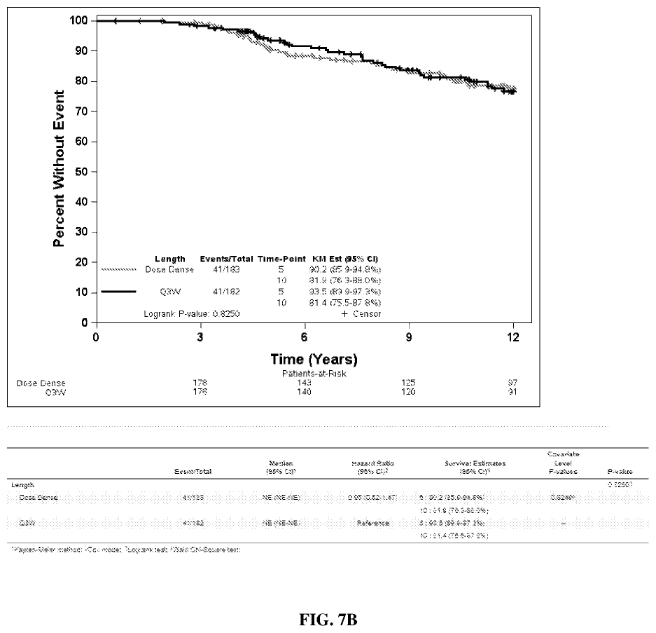

FIG. 6 demonstrates a predictive interaction for overall survival (interaction term p=0.03) favoring dose-intense chemotherapy if lower SET_{ER/PR} index, whereas the baseline prognostic index was not as predictive in the tested case (interaction p=0.45). The predictive interaction (SET_{ER/PR} index by dose-intensity) was independent of patients' menopausal status or HER2 status (whether defined using single gene expression or intrinsic subtype). From this, an exemplary cut point of SET_{ER/PR} index<0.75 can be identified to distinguish patients with cancer that has low SET_{ER/PR} index who may benefit from dose-intense chemotherapy (FIG. 7A) from patients whose cancer has high SET_{ER/PR} index and who may not benefit from dose-intense chemotherapy (FIG. 7B). These Kaplan Meier plots of Overall Survival (OS) of patients with ER-positive breast cancer in the CALGB 9741 trial demonstrate the predictive interaction between dose-intense chemotherapy and SET_{ER,PR} index using the cut point of 0.75. FIG. 7A demonstrates a significant benefit in overall survival (OS) after dose-intense chemotherapy when SET_{ER,PR} index in the breast cancer was less than 0.75, with hazard ratio 0.64 (95% confidence interval 0.42 to 0.99) and p value of 0.042. On the other hand, (FIG. 7B) demonstrates that patients did not benefit from dose-intense chemotherapy if the SET_{ER,PR} index in their breast cancer was 0.75 or greater: hazard ratio 0.95 (95% confidence interval 0.62 to 1.47) and p value of 0.825. This predictive interaction between SET_{ER/PR} index and chemotherapy treatment arm was retained after adjusting for HER2 status, defined either by intrinsic subtype (P_{interaction}=0.099 for DFS) or ERBB2 gene expression level from the SET2,3 assay (P_{interaction}=0.038).

Overall, low SET_{ER/PR} index (a component of the Set2,3 index) also predicted benefit from dose-intense chemotherapy (anthracycline-paclitaxel) in the CALGB 9741 trial, whereas the extent of disease and proliferation did not (BPI, ROR-PT). On the other hand, SET_{ER/PR} index had not predicted benefit from conventional anthracycline chemotherapy in the S8814 trial. This difference might be attributable to dosing schedule, inclusion of paclitaxel, or both, however it does suggest that the predictions have regimen-specificity. The finding that low level of endocrine-related transcriptional activity (that is not proliferation-related) measured by SET_{ER/PR} index significantly predicted survival benefit from dose-intense chemotherapy, versus conventional dosing would still fit with the original scientific premise of dose-intense chemotherapy in C9741 (based on the principles of low-volume Gompertzian growth kinetics after each treatment cycle) if endocrine-related transcription imparts a level of protection that enables cancer cells to recover from each cytotoxic treatment. Although both paclitaxel and anthracycline regimens were dose-intense in C9741, the prediction by SET_{ER/PR} index might be attributable to dose-density of taxane treatments. For example, MAPT, that encodes tau protein, is a gene in the SET_{ER/PR} index. Tau protein inhibits the binding site of paclitaxel within beta tubulin, and tau expression in breast cancer is associated with lower rates of pathologic response to paclitaxel.

### Example 4: Prognosis Associated with SET_{ER/PR} Index and Set2,3 Indexes According to Type of Adjuvant Chemotherapy Regimen

The preliminary data from retrospective cohort studies (shown in Table 9) suggests that the prognostic association of SET_{ER/PR} index in HR+/HER2− cancers might be different for dose-intense paclitaxel (ddT or wT) combined with an anthracycline (e.g., ddAC/T or wT/FAC regimens) than it is for docetaxel combined with an anthracycline (e.g., FEC/D) or conventionally dosed q3 wk paclitaxel combined with an anthracycline (e.g., AC/T). If benefit from dose-intense paclitaxel regimens (wT/FAC, ddAC/T) is concentrated in the low range of SET_{ER/PR} index, it would abrogate the prognostic association with SET_{ER/PR} index. This suggests that patients whose cancer has low SET_{ER/PR} index might also benefit from weekly paclitaxel (at lower dose) as demonstrated for dose-intense paclitaxel (Table 9).

TABLE 9
Prognostic Association of SET_{ER/PR} Index (DFS)
by Chemotherapy Regimen, Then Adjuvant ET.

| Dose-intense Paclitaxel Regimens | | | | | Other Chemotherapy Regimens | | | | |
|---|---|---|---|---|---|---|---|---|---|
| | | | HR | | | | | HR | |
| Cohort | ChemoRx | N= | (95% CI) | P= | Cohort | ChemoRx | N= | (95% CI) | P= |
| C9741 | ddAC/T | 164 | 0.84 | 0.42 | C9741 | AC/T | 148 | 0.47 | <0.01 |
| | | | (0.54- | | | | | (0.31- | |
| | | | 1.29) | | | | | 0.72) | |
| TBCRC- | ddAC/T | 119 | 1.26 | 0.35 | BCRF | FEC/D | 109 | 0.42 | <0.01 |
| 14 | | | (0.77- | | | | | (0.30- | |
| | | | 2.06) | | | | | 0.66) | |
| MDACC | wT/FAC | 241 | 0.72 | 0.20 | | | | | |
| | | | (0.44- | | | | | | |
| | | | 1.19) | | | | | | |

On the other hand, the SET_{ER/PR} index was strongly prognostic when adjuvant chemotherapy was docetaxel or paclitaxel with conventional q3-weekly dosing (AC/T, FEC/D), similar to findings from the 88814 trial. It suggests that benefit from addition ofdocetaxel might be either unrelated to the SET_{ER} index, or it might be associated with a higher range of SET_{ER/PR} index, in which both docetaxel chemotherapy and endocrine therapy could improve prognosis.

TABLE 10
Additional Examples where predictive treatments tests were identified
by one or more tests described herein are tabulated below:

| | | | Benefit in | | | | |
|---|---|---|---|---|---|---|---|
| | | | ER+ in | | | | Interaction: |
| Clinical | Treatment | | overall | | | | Treatment by |
| Trial | Arms | Taxane | trial? | Biomarker | N= | Endpoint | Biomarker |
| ECOG E- | AT vs AC | D | ER+/PR− | RS? | 734 | RFI | NS |
| 2197 | | | | | | | |
| PACS-01 | FEC/T vs | D | No | RS | 530 | DR | NS (p = |
| | FEC | | | | | | 0.79) |
| NSABP | AC/T vs | P | Yes | RS | 1065 | DFS | NS (p = |
| B-28 | AC | | | | | | 0.65) |
| RxPonder | Chemo vs | 96% | Premenopausal | RS | 5018 | IDFS | NS (p = |
| | Not | | | | | | 0.35) |
| GEICAM/ | FEC/T vs | P | No | EP | 536 | DMFS | NS (p = |
| 9906 | FEC | | | | | | 0.70) |
| GEICAM/ | FEC/T vs | P | No | PAM50 * | 536 | DMFS | NS (p = |
| 9906 | FEC | | | | | | 0.45) |
| CALGB | ddAC/T vs | P | No | ROR-PT | 596 | DMFS | NS (p = |
| 9741 | AC/T | | | | | | 0.45) |
| CALGB | ddAC/T vs | P | No | SET2, 3 | 613 | OS | Yes p = |
| 9741 | AC/T | | | | | | 0.03 |
| CALGB | ddAC/T vs | P | No | SET_{ER/PR} | 613 | OS | Yes p = |
| 9741 | AC/T | | | | | | 0.03 |
| CALGB | ddAC/T vs | P | No | BPI (T, N, | 613 | OS | NS (p = |
| 9741 | AC/T | | | RNA4) | | | 0.45) |

This finding, some 25 years after dose-intense anthracycline/paclitaxel chemotherapy became a standard 3^{rd} generation adjuvant regimen for patients with high-risk ER-positive disease, challenges the long-held paradigm that more intense chemotherapy regimens should be selected based solely on stage and high proliferation or genomic risk score.

### Example 5—Gene Expression Measurement Methods

Total RNA was extracted from 41 formalin-fixed paraffin-embedded (FFPE) breast cancer samples using Norgen FFPE RNA purification Kit (Norgen, Thorold, Canada) as described before.¹⁸ DNase I treatment was applied during RNA extraction. RNA concentration was determined with a Nanodrop spectrophotometer (NanoDrop, Wilmington, DE), and its quality was assessed with an Agilent 2100 Bioanalyzer (Agilent Technologies, Santa Clara, CA).

QuantiGene Plex Assay. As reported previously¹⁸, the 31-gene custom QGP assay (Thermo Fisher Scientific, Waltham, MA) was performed following manufacturer's instruction. Briefly, 250 ng of purified RNA was hybridized for 20 hours at 54° C. in a shaking incubator with QGP probes and magnetic beads in a 96-well round bottom plate. After washing and incubation with QGP reagents, the plate was read using a Luminex 200 instrument (Thermo Fisher Scientific).

Real-time RT qPCR. The SET_{ER/PR} panel was customized on real-time qPCR using TaqMan Gene Expression Array Plates (Thermo Fisher Scientific). Following the 32-format for plate layout, 31 pre-designed TaqMan Gene Expression Assays (Table 1) tailored to the 31 SET_{ER/PR} genes were carefully selected and dried down into dedicated reaction wells of a 96-well plate, along with a manufacturing control assay (Human 18S), one assay per well. This systematic configuration ensured that each sample was allocated 32 wells, and each plate accommodated 3 samples/replicates.

300 ng of FFPE RNA was reverse transcribed in a 20 ul reaction using SuperScript IV VILO Master Mix with ezDNase Enzyme kit (Thermo Fisher Scientific) following the manufacturer's recommendations. An adjustment was made to the incubation step at 55° C., extending it to 90 minutes to enhance cDNA yield. Subsequently, the cDNA was combined with TaqMan Fast Advanced Master Mix for qPCR (Thermo Fisher Scientific) to achieve a total master mix volume of 1020 μl. This mix was then loaded into a custom TaqMan Array Plate with 10 μl aliquots per well, corresponding to 3 ng (RNA-equivalent) per gene, and each sample was subjected to 3 technical replicates. The real-time qPCR was performed on a QuantStudio 3 Systems (Thermo Fisher Scientific) following standard cycling conditions over a total of 40 cycles.

### 1. Gene Expression Data Analysis and SET_{ER/PR} Calculation

### QGP Data Analysis

The SET_{ER/PR} index calculation used in the QGP panel, defined as:

$${SET}_{ER / PR} = \frac{{\sum }_{i = 2}^{18} {T}_{i}}{i} - \frac{{\sum }_{j = 1}^{10} {R}_{j}}{j} + 2 \qquad ( 1 )$$

where Tᵢ is log 2 of the ith expression of the 18 informative genes and Rⱼ is the log 2 of the jth expression of the 10 reference genes.³⁰
Real-Time RT qPCR Data Analysis

In order to calculate the index using data reported by RT qPCR, the assumption that RT qPCR operated close to theoretical efficiency was used:

$${[ cDNA ]}_{n} = {[ cDNA ]}_{0} * {2}^{n} \qquad ( 2 )$$

where [cDNA]ₙ is the concentration of a gene with an initial concentration of [cDNA]₀ after n cycles (3, Gevertz paper). Given that CT is the number of cycles it takes for the amplification of a gene, [DNA]ₙ, to reach a fixed threshold, the SET_{ER/PR} calculation becomes:

$${SET}_{ER / PR} = \frac{{\sum }_{j = 1}^{10} {CT}_{Rj}}{10} - \frac{{\sum }_{i = 1}^{18} {CT}_{Ti}}{18} + 2 \qquad ( 3 )$$

where CT_{Rj} and CT_{Ti} are the number of cycles for the 10 reference genes and 18 informative genes, respectively, to reach the threshold.

### Statistical Analysis

Statistical analysis was performed using the programming language R. Agreement between RT qPCR with QGP data as the standard was measured with Lin's concordance correlation coefficient (CCC). The relationship between the two platforms was modeled using linear regression. A standard Bland-Altman plot was used to visualize the comparison of SET_{ER/PR} index between RT qPCR and QGP. An extended Bland-Altman plot was used to visualize the stability of the technical replicates with three data points per sample. The median of the RT qPCR technical replicates of each sample was used when comparing to QGP data.

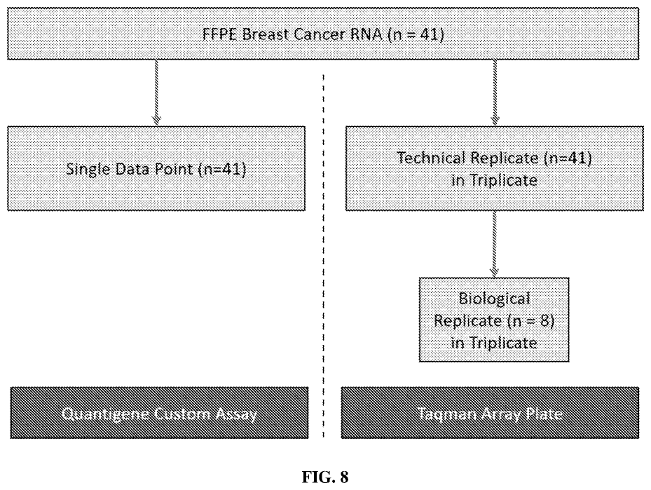

Study Design. Samples were measured in RT qPCR with 31 genes as technical triplicates by repeating the extraction from FFPE tissue sections from breast cancers (n=41). The same samples were measured in QGP 31-Plex as single data points (n=41). A subset of the samples measured as biological triplicates by repeating cDNA synthesis from the same RNA source (n=8) (FIG. 8).

Performance of RT qPCR. The platform's reported Ct of genes ranged from 23.7 to 40 cycles with a mean and median of 30.23 and 30.04 cycles, respectively. Average SET_{ER/PR} index ranged from −2.94 to 3.07.

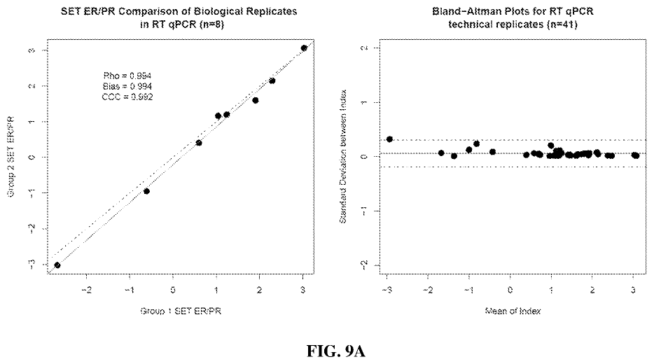

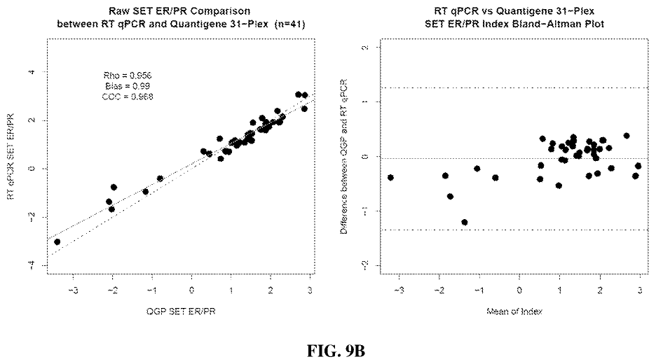

Stability of replicates from RT qPCR. Biological replicates (n=8) have high concordance (CCC 0.992) (FIG. 9A, Left). The standard deviation of SET_{ER/PR} index for technical replicates range from 0.01 to 0.32 with an average standard deviation of 0.06 (FIG. 9A, Right). Standard deviation decreases with higher SET_{ER/PR} values (FIG. 9A, Right).

Accuracy of RT qPCR compared to QGP. SET_{ER/PR} index concordance between the QGP assay and RT qPCR was high (CCC 0.968) with low scatter (Rho 0.956) and low bias (Bias 0.990) (FIG. 9B, Left). Differences between the two platforms decrease with higher mean SET_{ER/PR} values (FIG. 9B, Right).

TABLE 11
Assays selected for the custom Taqman array
plate for SET ERPR index measurement.

| | Applied Biosystems |
|---|---|
| Gene Name | TaqMan Assay ID |
| 18 SET index genes | |
| SLC39A6 | Hs00202392_m1 |
| STC2 | Hs01063215_m1 |
| CA12 | Hs01080909_m1 |
| PDZK1 | Hs00420042_m1 |
| NPY1R | Hs00702150_s1 |
| MAPT | Hs00213491_m1 |
| CD2 | Hs00233515_m1 |
| QDPR | Hs01077088_m1 |
| AZGP1 | Hs00426651_m1 |
| ADCY1 | Hs00299832_m1 |
| CD3D | Hs00174158_m1 |
| NAT1 | Hs00265080_s1 |
| MRPS30 | Hs00169612_m1 |
| DNAJC12 | Hs01111685_m1 |
| SCUBE2 | Hs00221277_m1 |
| MAPT | Hs00902194_m1 |
| ABAT | Hs00252934_s1 |
| KCNE4 | Hs00298953_m1 |
| ESR1 | Hs01046816_m1 |
| 10 reference genes | |
| LDHA | Hs01378790_g1 |
| ATP5J2 | Hs00934710_m1 |
| VDAC2 | Hs00748551_s1 |
| DARS | Hs00154683_m1 |
| UGP2 | Hs00198879_m1 |
| UBE2Z | Hs00225039_m1 |
| AK2 | Hs01123132_g1 |
| WIPF2 | Hs00370193_m1 |
| APPBP2 | Hs00197271_m1 |
| TRIM2 | Hs00991875_m1 |
| 3 breast cancer genes | |
| ERBB2 | Hs01001580_m1 |
| PGR | Hs01556702_m1 |
| AURKA | Hs00269212_m1 |

TABLE 12
Detected locations of SET index targets on different platforms.

| | | Affymetrix | | QuantiGene | Targeted | | |
|---|---|---|---|---|---|---|---|
| | | U133A Microarray | | Plex | RNAseq | | |
| | | | ProbeSet | Probe | Amplicon | | Amplicon |
| | | | location | location | location | Taqman | location |
| | | | on the | on the | on the | Real-time | on the |
| | RefSeq | ProbeSet | Specified | Specified | Specified | qPCR | Specified |
| Gene Symbol | Transcript ID | ID | Transcript | Transcript | Transcript | Assay ID | Transcript |
| SLC39A6 | NM_012319 | 202089_s_at | 2306-2651 | 341-892 | 1640-1815 | Hs00202392_m1 | 1359-1499 |
| STC2 | NM_003714 | 203438_at | 3688-4213 | 4626-5127 | 244-437 | Hs01063215_m1 | 1742-1882 |
| CA12 | NM_001218 | 204508_s_at | 3109-3530 | 1147-1752 | 204-397 | Hs01080909_m1 | 1312-1452 |
| PDZK1 | NM_002614 | 205380_at | 1630-2041 | 1110-1563 | 79-251 | Hs00420042_m1 | 299-439 |
| NPY1R | NM_000909 | 205440_s_at | 2264-2632 | 1763-2416 | 1308-1523 | Hs00702150_s1 | 1506-1646 |
| CD2 | NM_001767 | 205831_at | 1051-1478 | 995-1520 | 731-898 | Hs00233515_m1 | 22-162 |
| QDPR | NM_000320 | 209123_at | 809-1253 | 748-1293 | 593-791 | Hs01077088_m1 | 655-795 |
| AZGP1 | NM_001185 | 209309_at | 580-1121 | 293-805 | 572-774 | Hs00426651_m1 | 354-494 |
| ADCY1 | NM_021116 | 213245_at | 12091-12612 | 1338-1734 | 3110-3306 | Hs00299832_m1 | 971-1111 |
| CD3D | NM_000732 | 213539_at | 242-660 | 3-469 | 471-664 | Hs00174158_m1 | 126-266 |
| NAT1 | NM_000662 | 214440_at | 842-1340 | 644-1313 | 240-437 | Hs00265080_s1 | 865-1005 |
| MRPS30 | NM_016640 | 218398_at | 865-1294 | 832-1236 | 663-859 | Hs00169612_ml | 567-707 |
| DNAJC12 | NM_021800 | 218976_at | 631-1190 | 64-483 | 313-501 | Hs01111685_m1 | 596-736 |
| SCUBE2 | NM_020974 | 219197_s_at | 2565-3073 | 1620-2059 | 2778-2964 | Hs00221277_ml | 519-659 |
| MAPT | NM_005910 | 206401_s_at | 824-1455 | 1181-1481; | 1062-1213 | Hs00902194_m1 | 1367-1507 |
| | | | | 2952-3371; | | | |
| | | | | 4916-5359 | | | |
| ABAT | NM_000663 | 209459_s_at | 4247-4953 | 4497-4902 | 849-1047 | Hs00252934_s1 | 2142-2282 |
| KCNE4 | NM_080671 | 222379_at | 2458-2823 | 1405-1777 | 1315-1477 | Hs00298953_m1 | 278-418 |
| ESR1 | NM_000125 | 205225_at | 5756-6286 | 5538-6160 | 5225-5417 | Hs01046816_m1 | 1399-1539 |
| LDHA | NM_005566 | 200650_s_at | 1109-1533 | 67-610 | 799-989 | Hs01378790_g1 | 329-469 |
| ATP5J2 | NM_004889 | 202961_s_at | 82-400 | 36-420 | 31-230 | Hs00934710_m1 | 312-452 |
| VDAC2 | NM_003375 | 211662_s_at | 1128-1607 | 645-1326 | 1182-1353 | Hs00748551_s1 | 1502-1642 |
| DARS | NM_001349 | 201623_s_at | 1149-1692 | 1177-1649 | 1443-1633 | Hs00154683_m1 | 1559-1699 |
| UGP2 | NM_006759 | 205480_s_at | 1444-1923 | 1099-1613 | 1654-1810 | Hs00198879_m1 | 1653-1793 |
| UBE2Z | NM_023079 | 217750_s_at | 2245-2959 | 2477-2962 | 718-917 | Hs00225039_m1 | 961-1101 |
| AK2 | NM_001625 | 212175_s_at | 3454-3946 | 339-702 | 435-620 | Hs01123132_g1 | 512-652 |
| WIPF2 | NM_133264 | 212050_at | 2659-3117 | 4693-5083 | 1162-1314 | Hs00370193_m1 | 1345-1485 |
| APPBP2 | NM_006380 | 202631_at | 1861-2310 | 1301-1767 | 1724-1888 | Hs00197271_m1 | 1747-1887 |
| TRIM2 | NM_015271 | 202342_s_at | 6210-6508 | 931-1275 | 851-1010 | Hs00991875_ml | 263-403 |
| ERBB2 | NM_004448 | 216836_s_at | 3849-4402 | 1041-1576 | 1011-1164 | Hs01001580_m1 | 1928-2068 |
| AURKA | NM_003600 | 208079_s_at | 1582-2224 | 435-835 | 1004-1194 | Hs01582073_m1 | 1026-1176 |
| PGR | NM_000926 | 208305_at | 3034-3504 | 2609-3194 | 3320-3517 | Hs01556702_m1 | 2463-2603 |

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are snot to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Citron M L, Berry D A, Cirrincione C, et al. Randomized trial of dose-intense versus conventionally scheduled and sequential versus concurrent combination chemotherapy as postoperative adjuvant treatment of node-positive primary breast cancer: first report of Intergroup Trial C9741/Cancer and Leukemia Group B Trial 9741. J Clin Oncol 2003; 21:1431-9.
2. Berry D A, Cirrincione C, Henderson I C, et al. Estrogen-receptor status and outcomes of modern chemotherapy for patients with node-positive breast cancer. JAMA 2006; 295:1658-67.
3. Bonilla L, Ben-Aharon I, Vidal L, Gafter-Gvili A, Leibovici L, Stemmer S M. Dose-intense chemotherapy in nonmetastatic breast cancer: a systematic review and meta-analysis of randomized controlled trials. J Natl Cancer Inst 2010; 102:1845-54.
4. Goldvaser H, Majeed H, Ribnikar D, et al. Influence of control group therapy on the benefit from dose-intense chemotherapy in early breast cancer: a systemic review and meta-analysis. Breast Cancer Res Treat 2018; 169:413-25.
5. Norton L. Theoretical concepts and the emerging role of taxanes in adjuvant therapy. Oncologist 2001; 6 Suppl 3:30-5.
6. Liu M C, Pitcher B N, Mardis E R, et al. PAM50 gene signatures and breast cancer prognosis with adjuvant anthracycline- and taxane-based chemotherapy: correlative analysis of C9741 (Alliance). NPJ Breast Cancer 2016; 2.
7. Conte B, Bruzzone M, Lambertini M, et al. Effect of dose-intense adjuvant chemotherapy in hormone receptor positive/HER2-negative early breast cancer patients according to immunohistochemically defined luminal subtype: an exploratory analysis of the GIM2 trial. Eur J Cancer 2020; 136:43-51.
8. Martin M, Prat A, Rodriguez-Lescure A, et al. PAM50 proliferation score as a predictor of weekly paclitaxel benefit in breast cancer. Breast Cancer Res Treat 2013; 138:457-66.
9. Martin M, Brase J C, Calvo L, et al. Clinical validation of the EndoPredict test in node-positive, chemotherapy-treated ER+/HER2− breast cancer patients: results from the GEICAM 9906 trial. Breast Cancer Res 2014; 16:R38.
10. Martin M, Brase J C, Ruiz A, et al. Prognostic ability of EndoPredict compared to research-based versions of the PAM50 risk of recurrence (ROR) scores in node-positive, estrogen receptor-positive, and HER2-negative breast cancer. A GEICAM/9906 sub-study. Breast Cancer Res Treat 2016; 156:81-9.
11. Penault-Llorca F, Filleron T, Asselain B, et al. The 21-gene Recurrence Score(R) assay predicts distant recurrence in lymph node-positive, hormone receptor-positive, breast cancer patients treated with adjuvant sequential epirubicin- and docetaxel-based or epirubicin-based chemotherapy (PACS-01 trial). BMC Cancer 2018; 18:526.
12. Mamounas E P, Tang G, Paik S, et al. 21-Gene Recurrence Score for prognosis and prediction of taxane benefit after adjuvant chemotherapy plus endocrine therapy: results from NSABP B-28/NRG Oncology. Breast Cancer Res Treat 2018; 168:69-77.
13. Kalinsky K, Barlow W E, Gralow J R, et al. 21-Gene Assay to Inform Chemotherapy Benefit in Node-Positive Breast Cancer. N Engl J Med 2021; 385:2336-47.
14. Du L, Yau C, Brown-Swigart L, et al. Predicted sensitivity to endocrine therapy for stage II-III hormone receptor-positive and HER2-negative (HR+/HER2−) breast cancer before chemo-endocrine therapy. Ann Oncol 2021; 32:642-51.
15. Speers C W, Symmans W F, Barlow W E, et al. Evaluation of the Sensitivity to Endocrine Therapy Index and 21-Gene Breast Recurrence Score in the SWOG S8814 Trial. Journal of Clinical Oncology 2023; 0:JCO.22.01499.
16. Suman V J, Du L, Hoskin T, et al. Evaluation of Sensitivity to Endocrine Therapy Index (SET2,3) for Response to Neoadjuvant Endocrine Therapy and Longer-Term Breast Cancer Patient Outcomes (Alliance Z1031). Clin Cancer Res 2022; 28:3287-95.
17. Sinn B V, Fu C, Lau R, et al. SET-ER/PR—A robust 18-gene Predictor for Sensitivity to Endocrine Therapy for Metastatic Breast Cancer. NPJ Breast Cancer 2019; 5:1-8.
18. Lau R, Du L, Chen E, et al. Technical validity of a customized assay of sensitivity to endocrine therapy using sections from fixed breast cancer tissue. Clin Chem 2020; 66:934-45.
19. Bossuyt V, Lau R, Young B, et al. Intra- and Interlaboratoiy Reproducibility of the Sensitivity to Endocrine Therapy Assay for Stage I I/III Breast Cancer. Clin Chem 2021; 67:1240-8.
20. User Manual: QuantiGene Plex Assay. 2014.
21. QuantiGene Assays For Analysis of Gene Expression or Copy Number Variation: Assays Application Guide. 2017. (Accessed Aug. 21, 2020.
22. Rouzier R, Rajan R, Wagner P, et al. Microtubule-associated protein tau: a marker of paclitaxel sensitivity in breast cancer. Proc Natl Acad Sci USA 2005; 102:8315-20.
23. Pusztai L, Jeong J H, Gong Y, et al. Evaluation of microtubule-associated protein-Tau expression as a prognostic and predictive marker in the NSABP-B 28 randomized clinical trial. J Clin Oncol 2009; 27:4287-92.
24. Sparano J A, Wang M, Martino S, et al. Weekly paclitaxel in the adjuvant treatment of breast cancer. N Engl J Med 2008; 358:1663-71.
25. Sparano J A, Zhao F, Martino S, et al. Long-Term Follow-Up of the E1199 Phase III Trial Evaluating the Role of Taxane and Schedule in Operable Breast Cancer. J Clin Oncol 2015; 33:2353-60.
26. Allison K H, Hammond M E H, Dowsett M, et al. Estrogen and Progesterone Receptor Testing in Breast Cancer: ASCO/CAP Guideline Update. J Clin Oncol 2020; 38:1346-66.
27. Early Breast Cancer Trialists' Collaborative G. Effects of chemotherapy and hormonal therapy for early breast cancer on recurrence and 15-year survival: an overview of the randomised trials. Lancet 2005; 365:1687-717.
28. Gong Y, Yan K, Lin F, et al. Determination of oestrogen-receptor status and ERBB2 status of breast carcinoma: a gene-expression profiling study. Lancet Oncol 2007; 8:203-11.
29. Prat A, Carey L A, Adamo B, et al. Molecular features and survival outcomes of the intrinsic subtypes within HER2-positive breast cancer. J Natl Cancer Inst 2014; 106.
30. Sinn B V, Fu C, Lau R, Litton J, et al. SETER/PR: a robust 18-gene predictor for sensitivity to endocrine therapy for metastatic breast cancer. NPJ Breast Cancer. 2019 May 30; 5:16. PMID: 31231679; PMCID: PMC6542807.
31. Gevertz, J. L., Dunn, S. M. and Roth, C. M. (2005), Mathematical model of real-time PCR kinetics. Biotechnol. Bioeng., 92: 346-355.

What is claimed is:

1. A method for treating cancer in a subject comprising:
(a) obtaining a biological sample from the subject;
(b) extracting a plurality of molecules from the biological sample;
(c) quantitating the expression levels of at least one hormone receptor gene(s) selected from the group consisting of ABAT, ADCY1, AZGP1, CA12, CD2, CD3D, DNAJC12, ESR1, KCNE4, MAPT, MRPS30, NAT1, NPY1R, PDZK1, QDPR, SCUBE2, SLC39A6, STC2 in the plurality of nucleic acids by detecting and quantitating one or more RNA transcripts associated with the at least one hormone receptor gene;
(d) quantitating the expression levels of at least one reference gene selected from the group consisting of AK2, APPBP2, ATP5J2, DARS, LDHA, TRIM2, UBE2Z, UGP2, VDAC2, WIPF2 in the plurality of nucleic acids by detecting and quantitating one or more RNA transcripts associated with the at least one reference gene;
(e) calculating a cut-off level by the formula
$$Cutoff = \frac{{\sum }_{i = 1}^{18} {T}_{i}}{i} - \frac{{\sum }_{j = 1}^{10} {R}_{j}}{j} + 2$$
where Tᵢ is the expression of the ith of the set of genes and Rⱼ the expression of the jth of the set of reference genes; and
(f) administering a dose-intense chemotherapy regimen to a subject with a cut-off less than 0.75, or administering a conventional chemotherapy regimen or other anti-cancer therapy to a subject with a cut-off level equal or greater than 0.75.

2. The method of claim 1, further comprising determining a molecular subtype of the biological sample to obtain an RNA4 risk score.

3. The method of claim 2, wherein the determining the molecular subtype of the biological sample comprises quantitating the expression levels of one or more of ESR1, PGR, ERBB2, and AURKA in the plurality of nucleic acids by detecting and quantitating one or more RNA transcripts associated with the molecular subtype.

4. The method of claim 3, wherein the expression levels of the one or more of the ESR1, PGR, ERBB2, and AURKA genes are normalized by subtracting the mean of the 10 reference genes and adding the constant of 10.

5. The method of claim 4, wherein the RNA4 risk score is calculated as the sum of risk scores for each of the expression levels of ESR1, PGR, ERBB2, and AURKA.

6. The method of any one of claims 1-5, wherein the cancer is breast cancer.

7. The method of claim 6, wherein the breast cancer is metastatic breast cancer.

8. The method of any one of claims 1-7, further determining whether the subject is positive for estrogen receptors (ER+), progesterone receptors (PR+) or both (ER+/PR+).

9. The method of any one of claims 1-8, further comprising determining a pT risk score.

10. The method of claim 9, wherein the pT risk score is calculated from largest pathological tumor dimension and assigned score of zero if ≤10 mm, linearly scaled to increase 0.1 units per 1 mm increase in tumor dimension in the range 0-3.0 if measuring 11-39 mm, assigned risk score 3.0 if ≥40 mm, and assigned risk score 3.0 if the tumor otherwise has stage category pT4.

11. The method of any one of claims 1-10, further comprising determining a pN risk score.

12. The method of claim 11, wherein the pN risk score is calculated from the number of involved lymph nodes by the formula 0.5 units per node.

13. The method of claim 12, wherein the pN risk score is 5.0 if 10 or more nodes are involved or stage category pN3.

14. The method of claim 13, wherein a lymph node reported as isolated tumor cells (pNOi+) is considered to be negative for the pN risk score.

15. The method of any one of claims 1-14, further comprising determining a second cut-off level by the formula:
$$EAI Index Cutoff = 0.75 \times ( \frac{{\sum }_{i = 1}^{18} Ti}{i} - \frac{{\sum }_{j = 1}^{10} Rj}{j} + 2 ) + 0.51 \times ( 11 - ( pT + pN + RNA 4 ) ] \times 4 / 11 ) .$$

16. The method of claim 15, further comprising administering a dose-intense chemotherapy regimen to a subject determined to have an EAT Index cutoff less than 1.0, or administering a conventional chemotherapy regimen or other anti-cancer therapy to a subject determined to have an EAI Index cutoff greater than or equal to 1.0.

17. The method of claim 15, further comprising administering a dose-intense chemotherapy regimen to a subject determined to have an EAI Index cutoff less than 2.1, or administering a conventional chemotherapy regimen to a subject determined to have an EAI Index cutoff greater than or equal to 2.1.

18. The method of any one of claims 1-17, wherein the dose intense chemotherapy regimen comprises administering 20 mg/m² to 100 mg/m² of an anthracycline in combination with 30 mg/m² to 200 mg/m² of a taxane every two weeks.

19. The method of any one of claims 1-17, wherein the dose intense chemotherapy regimen comprises administering 60 mg/m² of an anthracycline in combination with 175 mg/m² of a taxane every two weeks.

20. The method of any one of claims 1-19, wherein at least 3 cycles of the dose intense chemotherapy regimen are administered.

21. The method of any one of claims 18-20, wherein the anthracycline is doxorubicin or epirubicin.

22. The method of any one of claims 18-21, wherein the taxane is paclitaxel, docetaxel, cabazitaxel, or abraxane.

23. The method of any one of claims 18-21, wherein the taxane is paclitaxel.

24. The method of any one of claims 1-23, wherein the dose intense chemotherapy further comprises administering cyclophosphamide.

25. The method of any one of claims 18-24, wherein the anthracycline, taxane and/or cyclophosphamide are administered sequentially.

26. The method of any one of claims 18-25, wherein the anthracycline, taxane and/or cyclophosphamide are administered concurrently.

27. The method of claim 24, wherein the cyclophosphamide is administered at a dose of 250 mg/m² to 1000 mg/m².

28. The method of claim 25, wherein the cyclophosphamide is administered at a dose of 600 mg/m².

29. The method of any one of claims 1-28, wherein the dose intense chemotherapy regimen comprises administering to the subject an amount of 30 mg/m² to 250 mg/m² of a taxane and/or 25 mg/m² to 200 mg/m² of an anthracycline at weekly intervals for at least 6 treatments.

30. The method of any one of claims 1-29, wherein the dose intense chemotherapy regimen comprises administering to the subject an amount of 30 mg/m² to 250 mg/m² of a taxane and/or 25 mg/m² to 200 mg/m² of an anthracycline every two weeks for at least 3 cycles.

31. The method of any one of claims 18-30, wherein the taxane is selected from the group consisting of paclitaxel, docetaxel, cabazitaxel, or abraxane.

32. The method of any one of claims 15-31, wherein if the cut-off level is equal to or greater than 2.1, then either an anthracycline chemotherapy or a taxane chemotherapy is administered on a conventional schedule.

33. The method of any one of claims 15-32, wherein if the cut-off level is equal to or greater than 2.1, then a chemotherapy selected from the group consisting of paclitaxel, docetaxel, cabazitaxel, abraxane, doxorubicin, cyclophosphamide, or anthracycline is administered on a conventional schedule.

34. The method of claim 32 or 33, wherein the conventional schedule comprises administering to the subject an amount of 30 mg/m² to 250 mg/m² of a taxane and/or 25 mg/m² to 200 mg/m² of an anthracycline every three weeks for at least 3 cycles.

35. The method of any one of claims 1-34, wherein the biological sample is a tumor biopsy.

36. The method of any one of claims 1-35, wherein the biological sample is formalin-fixed or a parafilm-embedded tissue biopsy.

37. The method of any one of claims 1-36, wherein quantitating comprises performing reverse transcription-quantitative real-time PCR (RT-qPCR), microarray analysis, amplification-free nucleic acid analysis, picodroplet targeting and reverse transcription, or RNA sequencing.

38. The method of any one of claims 1-37, wherein quantifying comprises performing RT-qPCR.

39. The method of any one of claims 1-37, wherein quantifying comprises performing nanostring nCounter.

40. The method of any one of claims 1-37, wherein quantifying comprising performing targeted RNA sequencing.

41. The method of any one of claims 1-40, further comprising administering an additional anti-cancer therapy.

42. The method of claim 41, wherein the anti-cancer therapy increases the effectiveness of the dose-intense chemotherapy.

43. The method of claim 41, wherein the additional anti-cancer therapy is a radiation therapy, hormone therapy, immunotherapy or cytokine therapy.

44. The method of claim 41, wherein the additional anti-cancer therapy is an immunotherapy.

45. The method of claim 41, wherein the immunotherapy comprises an immune checkpoint inhibitor.

46. The method of claim 45, wherein the immune checkpoint inhibitor is an inhibitor of CTLA-4, PD-1, PD-L1, PD-L2, LAG-3, BTLA, B7H3, B7H4, TIM3, KIR, or A2aR.

47. The method of claim 45, wherein the immune checkpoint inhibitor is an anti-CTLA-4, anti-PD-1, or anti-PD-L1 antibody.

48. The method of claim 47, wherein the anti-PDL1 antibody is atezolizumab, durvalumab, and avelumab.

49. The method of claim 47, wherein the anti-PD1 antibody is nivolumab, pembrolizumab, pidilizumab, KEYTRUDA®, AMP-514, REGN2810, CT-011, BMS 936559, MPDL3280A or AMP-224.

50. The method of claim 47, wherein the anti-CTLA-4 antibody is tremelimumab, YERVOY®, or ipilimumab.

51. A method for predicting effectiveness of dose-intense chemotherapy regimens on a subject, the method comprising the steps of:
determining whether the subject is positive for estrogen receptors (ER+), progesterone receptors (PR+) or both (ER+/PR+) by:
obtaining a biological sample from the subject;
extracting a plurality of molecules from the biological sample;
performing an assay that:
quantitates the expression levels of at least one hormone receptor gene(s) selected from the group consisting of ABAT, ADCY1, AZGP1, CA12, CD2, CD3D, DNAJC12, ESR1, KCNE4, MAPT, MRPS30, NAT1, NPY1R, PDZK1, QDPR, SCUBE2, SLC39A6, STC2 in the plurality of nucleic acids by detecting and quantitating one or more RNA transcripts associated with the at least one hormone receptor gene;
quantitates the expression levels of at least one reference gene selected from the group consisting of AK2, APPBP2, ATP5J2, DARS, LDHA, TRIM2, UBE2Z, UGP2, VDAC2, WIPF2 in the plurality of nucleic acids by detecting and quantitating one or more RNA transcripts associated with the at least one reference gene;
provides the expression levels of the detected genes with the formula expression levels of
$$ER + , PR + , or both transcripts = \frac{{\sum }_{i = 1}^{18} Ti}{i} - \frac{{\sum }_{j = 1}^{10} Rj}{j} + 2$$
where Tᵢ is the expression of the ith of the set of genes and Rⱼ the expression of the jth of the set of reference genes;
if the subject has a cut-off level that is lesser than 1.0, then internally administering a dose-intense chemotherapy regimen to the patient;
if the subject has a cut-off level that is equal then or greater than 1.0, then administering a conventional chemotherapy regiment to the patient or not administering any chemotherapy to the patient.

52. The method of claim 51, wherein the performing of the assay further comprises determining a molecular subtype of the biological sample.

53. The method of claim 52, wherein the determining the molecular subtype of the biological sample comprises quantitating the expression levels of one or more of ESR1, PGR, ERBB2, and AURKA in the plurality of nucleic acids by detecting and quantitating one or more RNA transcripts associated with the molecular subtype.

54. The method of claim 53, wherein the expression levels of the one or more of the ESR1, PGR, ERBB2, and AURKA genes are normalized by subtracting the mean of the 10 reference genes and adding the constant of 10.

55. The method of claim 51, wherein the performing of the assay further comprises determining a pT risk score.

56. The method of claim 55, wherein the pT risk score is calculated from largest pathological tumor dimension and assigned score of zero if 10 mm, linearly scaled to the range 0-3.0 if measuring 11-39 mm, assigned risk score 3.0 if ≥40 mm, and assigned risk score 3.0 if the tumor otherwise has stage category pT4.

57. The method of claim 51, wherein the performing of the assay further comprises determining a pN risk score.

58. The method of claim 57, wherein the pN risk score is calculated from the number of involved lymph nodes (0.5 units per node) and assigned score of 5.0 if 10 or more nodes are involved or otherwise stage category pN3.

59. The method of claim 58, wherein a lymph node reported as isolated tumor cells (pNOi+) is considered to be negative for the pN risk score.

60. The method of any one of claims 51-59, further comprising determining a second cut-off level by the formula:
$$EAI Index Cutoff = 0.75 \times ( \frac{{\sum }_{i = 1}^{18} Ti}{i} - \frac{{\sum }_{j = 1}^{10} Rj}{j} + 2 ) + 0.51 \times ( 11 - ( pT + pN + RNA 4 ) ] \times 4 / 11 ) .$$

61. The method of claim 51, wherein the dose intense chemotherapy regimen comprises administering to the subject an amount of 30 mg/m² to 250 mg/m² of a taxane at weekly intervals for at least 6 treatments.

62. The method of claim 61, wherein the dose intense chemotherapy regimen comprises administering to the subject an amount of 30 mg/m² b.i.d to 250 mg/m² b.i.d of a taxane every two weeks for at least 3 cycles.

63. The method of claim 62, wherein the dose intense chemotherapy regimen comprises administering to the subject an amount of 30 mg/m² b.i.d to 250 mg/m² b.i.d of a taxane every three weeks for at least 3 cycles.

64. The method of any one of claims 61-63, wherein the taxane is selected from the group consisting of paclitaxel, docetaxel, cabazitaxel, or abraxane.

65. The method of claim 51, wherein if the cut-off level is lesser than 2.1, then internally administering a dose-intense paclitaxel chemotherapy regimen to the patient.

66. The method of claim 51, wherein the dose intense chemotherapy regimen comprises administering to the subject an amount of 25 mg/m² b.i.d to 200 mg/m² b.i.d of an anthracycline every week for a period of at least 6 weeks.

67. The method of claim 66, wherein the dose intense chemotherapy regimen comprises administering to the subject an amount of 25 mg/m² b.i.d to 200 mg/m²b.i.d of an anthracycline every two weeks for a period of at least 6 weeks.

68. The method of claim 66, wherein the dose intense chemotherapy regimen comprises administering to the subject an amount of 25 mg/m² b.i.d to 200 mg/m²b.i.d of an anthracycline every three weeks for a period of at least 6 weeks.

69. The method of claim 51, wherein if the cut-off level is equal then or greater than 2.1, then a dose-intense chemotherapy regimen is not administered to the subject.

70. The method of claim 51, wherein if the cut-off level is equal then or greater than 2.1, then either an anthracycline chemotherapy or a taxane chemotherapy is administered on a conventional schedule.

71. The method of claim 51, wherein if the cut-off level is equal then or greater than 2.1, then a chemotherapy selected from the group consisting of paclitaxel, docetaxel, cabazitaxel, abraxane, doxorubicin, cyclophosphamide, or anthracycline is administered on a conventional schedule.

72. The method of claim 51, wherein the biological sample is a tumor biopsy.

73. The method of claim 51, wherein the biological sample is formalin-fixed or a parafilm-embedded tissue biopsy.

74. The method of claim 51, wherein the assay that quantitates the expression levels of the plurality of nucleic acids comprises performing reverse transcription-quantitative real-time PCR (RT-qPCR), microarray analysis, amplification-free nucleic acid analysis, picodroplet targeting and reverse transcription, or RNA sequencing.

75. The method of any one of claims 51-74, further comprising administering an additional anti-cancer therapy.

76. The method of claim 75, wherein the anti-cancer therapy increases the effectiveness of the dose-intense chemotherapy.

77. The method of claim 75, wherein the additional anti-cancer therapy is an radiation therapy, hormone therapy, immunotherapy or cytokine therapy.

78. The method of claim 75, wherein the immunotherapy comprises an immune checkpoint inhibitor.

79. The method of claim 78, wherein the immune checkpoint inhibitor is an inhibitor of CTLA-4, PD-1, PD-L1, PD-L2, LAG-3, BTLA, B7H3, B7H4, TIM3, KIR, or A2aR.

80. The method of claim 78, wherein the immune checkpoint inhibitor is an anti-CTLA-4, anti-PD-1, or anti-PD-L1 antibody.

81. The method of claim 80, wherein the anti-PDL1 antibody is atezolizumab, durvalumab, and avelumab.

82. The method of claim 80, wherein the anti-PD1 antibody is nivolumab, pembrolizumab, pidilizumab, KEYTRUDA®, AMP-514, REGN2810, CT-011, BMS 936559, MPDL3280A or AMP-224.

83. The method of claim 80, wherein the anti-CTLA-4 antibody is tremelimumab, YERVOY®, or ipilimumab.


SEQUENCE LISTING (434 sequences)

SEQ ID NO: 1 - DNA, 25 bp, synthetic construct
  source 1..25: mol_type = other DNA
tgcacttactgctggcttattcatg

SEQ ID NO: 2 - DNA, 25 bp, synthetic construct
  source 1..25: mol_type = other DNA
ggcttattcatgtatgttgctctgg

SEQ ID NO: 3 - DNA, 25 bp, synthetic construct
  source 1..25: mol_type = other DNA
gttgctctggttgatatggtacctg

SEQ ID NO: 4 - DNA, 25 bp, synthetic construct
  source 1..25: mol_type = other DNA
gctgcacaatgatgctagtgaccat

SEQ ID NO: 5 - DNA, 25 bp, synthetic construct
  source 1..25: mol_type = other DNA
agtgaccatggatgtagccgctggg

SEQ ID NO: 6 - DNA, 25 bp, synthetic construct
  source 1..25: mol_type = other DNA
tgtagccgctgggggtatttctttt

SEQ ID NO: 7 - DNA, 25 bp, synthetic construct
  source 1..25: mol_type = other DNA
tgctgggatgcttttgggttttgga

SEQ ID NO: 8 - DNA, 25 bp, synthetic construct
  source 1..25: mol_type = other DNA
gtcatagtttcagtaggtcataggg

SEQ ID NO: 9 - DNA, 25 bp, synthetic construct
  source 1..25: mol_type = other DNA
gtatgctgtactatgcagcgtttaa

SEQ ID NO: 10 - DNA, 25 bp, synthetic construct
  source 1..25: mol_type = other DNA
gaatattgctgtctgttacaaagtc

SEQ ID NO: 11 - DNA, 25 bp, synthetic construct
  source 1..25: mol_type = other DNA
tgtgcaattcaccggtattaccagt

SEQ ID NO: 12 - DNA, 25 bp, synthetic construct
  source 1..25: mol_type = other DNA
gtccacattcctgcaagcattgatt

SEQ ID NO: 13 - DNA, 25 bp, synthetic construct
  source 1..25: mol_type = other DNA
cctctacttgggctttatactgcat

SEQ ID NO: 14 - DNA, 25 bp, synthetic construct
  source 1..25: mol_type = other DNA
tttactcatgagccttcctttgagg

SEQ ID NO: 15 - DNA, 25 bp, synthetic construct
  source 1..25: mol_type = other DNA
caagatgatctgaacacctctcctt

SEQ ID NO: 16 - DNA, 25 bp, synthetic construct
  source 1..25: mol_type = other DNA
aacaccaggccagaactatagtcat

SEQ ID NO: 17 - DNA, 25 bp, synthetic construct
  source 1..25: mol_type = other DNA
aaaaacaatgcttccagtggccact

SEQ ID NO: 18 - DNA, 25 bp, synthetic construct
  source 1..25: mol_type = other DNA
tccagtggccacttcctaaggagga

SEQ ID NO: 19 - DNA, 25 bp, synthetic construct
  source 1..25: mol_type = other DNA
ctaaggaggaacaaccccgtctgat

SEQ ID NO: 20 - DNA, 25 bp, synthetic construct
  source 1..25: mol_type = other DNA
tctgatctcagaattggcaccacgt

SEQ ID NO: 21 - DNA, 25 bp, synthetic construct
  source 1..25: mol_type = other DNA
ggcaccacgtgagcttgctaagtga

SEQ ID NO: 22 - DNA, 25 bp, synthetic construct
  source 1..25: mol_type = other DNA
ggacctgtacattgtcacattgcat

SEQ ID NO: 23 - DNA, 25 bp, synthetic construct
  source 1..25: mol_type = other DNA
gggactgtctgtcaaaagactctgt

SEQ ID NO: 24 - DNA, 25 bp, synthetic construct
  source 1..25: mol_type = other DNA
gaccattgtacctggcacaagggct

SEQ ID NO: 25 - DNA, 25 bp, synthetic construct
  source 1..25: mol_type = other DNA
gagacttactgggaggtgcaagaca

SEQ ID NO: 26 - DNA, 25 bp, synthetic construct
  source 1..25: mol_type = other DNA
tgcaagacagtggcatttctcctct

SEQ ID NO: 27 - DNA, 25 bp, synthetic construct
  source 1..25: mol_type = other DNA
ctgagaccagacaaagcccgggagg

SEQ ID NO: 28 - DNA, 25 bp, synthetic construct
  source 1..25: mol_type = other DNA
gaaccaacactatcaatgtctttgc

SEQ ID NO: 29 - DNA, 25 bp, synthetic construct
  source 1..25: mol_type = other DNA
atgtctttgcaaatcctcacaggat

SEQ ID NO: 30 - DNA, 25 bp, synthetic construct
  source 1..25: mol_type = other DNA
cacaggattcctgtgggtccagctt

SEQ ID NO: 31 - DNA, 25 bp, synthetic construct
  source 1..25: mol_type = other DNA
gccagtactgtggtgtgtgagtctc

SEQ ID NO: 32 - DNA, 25 bp, synthetic construct
  source 1..25: mol_type = other DNA
gctcctaactctgctgcatggcaga

SEQ ID NO: 33 - DNA, 25 bp, synthetic construct
  source 1..25: mol_type = other DNA
gctgcatggcagatgcctaggtgga

SEQ ID NO: 34 - DNA, 25 bp, synthetic construct
  source 1..25: mol_type = other DNA
gtcaaaccatgactcgcacatggca

SEQ ID NO: 35 - DNA, 25 bp, synthetic construct
  source 1..25: mol_type = other DNA
aaagaacgggcccacagtacagcct

SEQ ID NO: 36 - DNA, 25 bp, synthetic construct
  source 1..25: mol_type = other DNA
atttgatagctgtttctgggtattt

SEQ ID NO: 37 - DNA, 25 bp, synthetic construct
  source 1..25: mol_type = other DNA
gtgacctgtttactgtctctttaga

SEQ ID NO: 38 - DNA, 25 bp, synthetic construct
  source 1..25: mol_type = other DNA
tcaccatgtgtgactgtcttctgtt

SEQ ID NO: 39 - DNA, 25 bp, synthetic construct
  source 1..25: mol_type = other DNA
ttatcatttgtcttacaggcggcta

SEQ ID NO: 40 - DNA, 25 bp, synthetic construct
  source 1..25: mol_type = other DNA
tacaggcggctattgcagacggcta

SEQ ID NO: 41 - DNA, 25 bp, synthetic construct
  source 1..25: mol_type = other DNA
gatttttttcatgtgatcttttcca

SEQ ID NO: 42 - DNA, 25 bp, synthetic construct
  source 1..25: mol_type = other DNA
ttccaagcttcaacttaacttaact

SEQ ID NO: 43 - DNA, 25 bp, synthetic construct
  source 1..25: mol_type = other DNA
gtatgatgatgtctcttacttctac

SEQ ID NO: 44 - DNA, 25 bp, synthetic construct
  source 1..25: mol_type = other DNA
ttacttctacaggttccttgagcac

SEQ ID NO: 45 - DNA, 25 bp, synthetic construct
  source 1..25: mol_type = other DNA
cagactgttcagtgtttgtcaagct

SEQ ID NO: 46 - DNA, 25 bp, synthetic construct
  source 1..25: mol_type = other DNA
gtgtttgtcaagcttctggtctaat

SEQ ID NO: 47 - DNA, 25 bp, synthetic construct
  source 1..25: mol_type = other DNA
agcttctggtctaatatgtactcga

SEQ ID NO: 48 - DNA, 25 bp, synthetic construct
  source 1..25: mol_type = other DNA
gaaagactttccgcttacaatttgt

SEQ ID NO: 49 - DNA, 25 bp, synthetic construct
  source 1..25: mol_type = other DNA
atatcgttttccatacagcagtgcc

SEQ ID NO: 50 - DNA, 25 bp, synthetic construct
  source 1..25: mol_type = other DNA
ttaactttcaatgtccatctttcaa

SEQ ID NO: 51 - DNA, 25 bp, synthetic construct
  source 1..25: mol_type = other DNA
gatacttcatatagcccattttaac

SEQ ID NO: 52 - DNA, 25 bp, synthetic construct
  source 1..25: mol_type = other DNA
tgtgtgacttgtggcgtcttataaa

SEQ ID NO: 53 - DNA, 25 bp, synthetic construct
  source 1..25: mol_type = other DNA
gttgtgtcatgttaatgtgcctaat

SEQ ID NO: 54 - DNA, 25 bp, synthetic construct
  source 1..25: mol_type = other DNA
tgtgcctaatttcatgtatcttgta

SEQ ID NO: 55 - DNA, 25 bp, synthetic construct
  source 1..25: mol_type = other DNA
ttctgaggagtctctttcagtttca

SEQ ID NO: 56 - DNA, 25 bp, synthetic construct
  source 1..25: mol_type = other DNA
agacctcgagttcagccaaaacctc

SEQ ID NO: 57 - DNA, 25 bp, synthetic construct
  source 1..25: mol_type = other DNA
ggcagcagaaaactcattgtcccct

SEQ ID NO: 58 - DNA, 25 bp, synthetic construct
  source 1..25: mol_type = other DNA
aaaagcactgtggatttctgccctc

SEQ ID NO: 59 - DNA, 25 bp, synthetic construct
  source 1..25: mol_type = other DNA
ctgatgtgcatatccgtacttccat

SEQ ID NO: 60 - DNA, 25 bp, synthetic construct
  source 1..25: mol_type = other DNA
gtacttccatgaggtgttttctgtg

SEQ ID NO: 61 - DNA, 25 bp, synthetic construct
  source 1..25: mol_type = other DNA
tgtgcagaacattgtcacctcctga

SEQ ID NO: 62 - DNA, 25 bp, synthetic construct
  source 1..25: mol_type = other DNA
gagtttttggtctcctcagagagct

SEQ ID NO: 63 - DNA, 25 bp, synthetic construct
  source 1..25: mol_type = other DNA
agagctccatcacaccagtaaggag

SEQ ID NO: 64 - DNA, 25 bp, synthetic construct
  source 1..25: mol_type = other DNA
aatcttagagatttcttgtcccctc

SEQ ID NO: 65 - DNA, 25 bp, synthetic construct
  source 1..25: mol_type = other DNA
tcccctctcaggtcatgtgtagatg

SEQ ID NO: 66 - DNA, 25 bp, synthetic construct
  source 1..25: mol_type = other DNA
gtctcactacaagcagcctatctgc

SEQ ID NO: 67 - DNA, 25 bp, synthetic construct
  source 1..25: mol_type = other DNA
gtcactaacctgtctcagtgtggcc

SEQ ID NO: 68 - DNA, 25 bp, synthetic construct
  source 1..25: mol_type = other DNA
ttctgtaacccctgtttgtggtacg

SEQ ID NO: 69 - DNA, 25 bp, synthetic construct
  source 1..25: mol_type = other DNA
atgcatttgctctcctaggacagtg

SEQ ID NO: 70 - DNA, 25 bp, synthetic construct
  source 1..25: mol_type = other DNA
aagacatgcgagactggtggcctgc

SEQ ID NO: 71 - DNA, 25 bp, synthetic construct
  source 1..25: mol_type = other DNA
gtggcctgcaaatagcatccgtcaa

SEQ ID NO: 72 - DNA, 25 bp, synthetic construct
  source 1..25: mol_type = other DNA
catccgtcaatctgtgttaactgca

SEQ ID NO: 73 - DNA, 25 bp, synthetic construct
  source 1..25: mol_type = other DNA
tagcacctgctatagcggtgtcatg

SEQ ID NO: 74 - DNA, 25 bp, synthetic construct
  source 1..25: mol_type = other DNA
cggtgtcatgttggatcgcttttgt

SEQ ID NO: 75 - DNA, 25 bp, synthetic construct
  source 1..25: mol_type = other DNA
ttgtgactgttcatctgtccttgac

SEQ ID NO: 76 - DNA, 25 bp, synthetic construct
  source 1..25: mol_type = other DNA
gacgtagttttcgcatccttgaatt

SEQ ID NO: 77 - DNA, 25 bp, synthetic construct
  source 1..25: mol_type = other DNA
aagcaaaactccatggactctgtcc

SEQ ID NO: 78 - DNA, 25 bp, synthetic construct
  source 1..25: mol_type = other DNA
tgcggaaatacctgaaatacagcaa

SEQ ID NO: 79 - DNA, 25 bp, synthetic construct
  source 1..25: mol_type = other DNA
acagcaaaaatatcctggaccggca

SEQ ID NO: 80 - DNA, 25 bp, synthetic construct
  source 1..25: mol_type = other DNA
aatatcctggaccggcaagatcctc

SEQ ID NO: 81 - DNA, 25 bp, synthetic construct
  source 1..25: mol_type = other DNA
aatggaaatggcacttaccagtcct

SEQ ID NO: 82 - DNA, 25 bp, synthetic construct
  source 1..25: mol_type = other DNA
tggaaatggcacttaccagtcctgg

SEQ ID NO: 83 - DNA, 25 bp, synthetic construct
  source 1..25: mol_type = other DNA
cagctaggaagcaagggttggaggc

SEQ ID NO: 84 - DNA, 25 bp, synthetic construct
  source 1..25: mol_type = other DNA
accacagaaatcacagtcaatggat

SEQ ID NO: 85 - DNA, 25 bp, synthetic construct
  source 1..25: mol_type = other DNA
gcctgtcttgagtagacttggaccc

SEQ ID NO: 86 - DNA, 25 bp, synthetic construct
  source 1..25: mol_type = other DNA
gtcttgagtagacttggacccaaaa

SEQ ID NO: 87 - DNA, 25 bp, synthetic construct
  source 1..25: mol_type = other DNA
tgagtagacttggacccaaaaaatc

SEQ ID NO: 88 - DNA, 25 bp, synthetic construct
  source 1..25: mol_type = other DNA
aaaaaatcatctcaccttgagccca

SEQ ID NO: 89 - DNA, 25 bp, synthetic construct
  source 1..25: mol_type = other DNA
ccccgctcaaacaacaatgtcctta

SEQ ID NO: 90 - DNA, 25 bp, synthetic construct
  source 1..25: mol_type = other DNA
ctcgtagtggtacattccattccta

SEQ ID NO: 91 - DNA, 25 bp, synthetic construct
  source 1..25: mol_type = other DNA
aaagttgtgtaagcgcctgcgttct

SEQ ID NO: 92 - DNA, 25 bp, synthetic construct
  source 1..25: mol_type = other DNA
cgttcttctgggtttggctagatag

SEQ ID NO: 93 - DNA, 25 bp, synthetic construct
  source 1..25: mol_type = other DNA
gatagggttgtgtccctctatggaa

SEQ ID NO: 94 - DNA, 25 bp, synthetic construct
  source 1..25: mol_type = other DNA
tttctcgcacaatacaactcactga

SEQ ID NO: 95 - DNA, 25 bp, synthetic construct
  source 1..25: mol_type = other DNA
aactcactgaggatgcttctgtaga

SEQ ID NO: 96 - DNA, 25 bp, synthetic construct
  source 1..25: mol_type = other DNA
taactcactctcactgggtaacttc

SEQ ID NO: 97 - DNA, 25 bp, synthetic construct
  source 1..25: mol_type = other DNA
acgggtctatttttatgtcaactga

SEQ ID NO: 98 - DNA, 25 bp, synthetic construct
  source 1..25: mol_type = other DNA
aacactgtagggtaccttccagtct

SEQ ID NO: 99 - DNA, 25 bp, synthetic construct
  source 1..25: mol_type = other DNA
gtgtttgcactatactttctggaat

SEQ ID NO: 100 - DNA, 25 bp, synthetic construct
  source 1..25: mol_type = other DNA
gggaacactgctctcagacattaca

SEQ ID NO: 101 - DNA, 25 bp, synthetic construct
  source 1..25: mol_type = other DNA
gactggacctgggaaaacgcatcct

SEQ ID NO: 102 - DNA, 25 bp, synthetic construct
  source 1..25: mol_type = other DNA
cgcatcctggacccacgaggaatat

SEQ ID NO: 103 - DNA, 25 bp, synthetic construct
  source 1..25: mol_type = other DNA
gaatctaccgtgcaagttcattatc

SEQ ID NO: 104 - DNA, 25 bp, synthetic construct
  source 1..25: mol_type = other DNA
gtgtggagctggatccagccaccgt

SEQ ID NO: 105 - DNA, 25 bp, synthetic construct
  source 1..25: mol_type = other DNA
agactggaaggctgtctggggctgc

SEQ ID NO: 106 - DNA, 25 bp, synthetic construct
  source 1..25: mol_type = other DNA
tgccgacacacaagctctgttgagg

SEQ ID NO: 107 - DNA, 25 bp, synthetic construct
  source 1..25: mol_type = other DNA
ggaatgaccaggtctatcagcccct

SEQ ID NO: 108 - DNA, 25 bp, synthetic construct
  source 1..25: mol_type = other DNA
agcccctccgagatcgagatgatgc

SEQ ID NO: 109 - DNA, 25 bp, synthetic construct
  source 1..25: mol_type = other DNA
tgctcagtacagccaccttggagga

SEQ ID NO: 110 - DNA, 25 bp, synthetic construct
  source 1..25: mol_type = other DNA
gaagcagccattaccaactgtacct

SEQ ID NO: 111 - DNA, 25 bp, synthetic construct
  source 1..25: mol_type = other DNA
gatggggttcactgtttggtgggct

SEQ ID NO: 112 - DNA, 25 bp, synthetic construct
  source 1..25: mol_type = other DNA
ttcaccctcacccataggagattca

SEQ ID NO: 113 - DNA, 25 bp, synthetic construct
  source 1..25: mol_type = other DNA
gaaagcttgtgcccaaacatggtga

SEQ ID NO: 114 - DNA, 25 bp, synthetic construct
  source 1..25: mol_type = other DNA
aatcttgtctatttgtcatccagct

SEQ ID NO: 115 - DNA, 25 bp, synthetic construct
  source 1..25: mol_type = other DNA
catccagctcaccagttatcaactg

SEQ ID NO: 116 - DNA, 25 bp, synthetic construct
  source 1..25: mol_type = other DNA
gttatcaactgacgacctatcatgt

SEQ ID NO: 117 - DNA, 25 bp, synthetic construct
  source 1..25: mol_type = other DNA
gacgacctatcatgtatcttctgta

SEQ ID NO: 118 - DNA, 25 bp, synthetic construct
  source 1..25: mol_type = other DNA
tatcatgtatcttctgtacccttac

SEQ ID NO: 119 - DNA, 25 bp, synthetic construct
  source 1..25: mol_type = other DNA
aatcctagacatcaaatcatttcac

SEQ ID NO: 120 - DNA, 25 bp, synthetic construct
  source 1..25: mol_type = other DNA
agatggcctgtggttatcttggaaa

SEQ ID NO: 121 - DNA, 25 bp, synthetic construct
  source 1..25: mol_type = other DNA
gttggtttattgttgaattcctaga

SEQ ID NO: 122 - DNA, 25 bp, synthetic construct
  source 1..25: mol_type = other DNA
tgttggctcaaaaactgcagatcct

SEQ ID NO: 123 - DNA, 25 bp, synthetic construct
  source 1..25: mol_type = other DNA
tgcagatccttgctgttacggtcac

SEQ ID NO: 124 - DNA, 25 bp, synthetic construct
  source 1..25: mol_type = other DNA
cacacccagtttcatctgttacctg

SEQ ID NO: 125 - DNA, 25 bp, synthetic construct
  source 1..25: mol_type = other DNA
gagctaatgctattgcaagcctttt

SEQ ID NO: 126 - DNA, 25 bp, synthetic construct
  source 1..25: mol_type = other DNA
agcctttttgcttggactggagcac

SEQ ID NO: 127 - DNA, 25 bp, synthetic construct
  source 1..25: mol_type = other DNA
agcagatgttactcgaccttttgtc

SEQ ID NO: 128 - DNA, 25 bp, synthetic construct
  source 1..25: mol_type = other DNA
ttgtctcccaggctgtgatcacaga

SEQ ID NO: 129 - DNA, 25 bp, synthetic construct
  source 1..25: mol_type = other DNA
tttcctttttctgctaccagctaaa

SEQ ID NO: 130 - DNA, 25 bp, synthetic construct
  source 1..25: mol_type = other DNA
actttggcactgactacacaagctg

SEQ ID NO: 131 - DNA, 25 bp, synthetic construct
  source 1..25: mol_type = other DNA
agctgatcaaaataaccctcgtaaa

SEQ ID NO: 132 - DNA, 25 bp, synthetic construct
  source 1..25: mol_type = other DNA
acttcagatagttcactttctactg

SEQ ID NO: 133 - DNA, 25 bp, synthetic construct
  source 1..25: mol_type = other DNA
cccaagcccctagagaagtcagtct

SEQ ID NO: 134 - DNA, 25 bp, synthetic construct
  source 1..25: mol_type = other DNA
agtctccccgcaaaattcagattct

SEQ ID NO: 135 - DNA, 25 bp, synthetic construct
  source 1..25: mol_type = other DNA
tgtgaatggttggcaccttcgtttc

SEQ ID NO: 136 - DNA, 25 bp, synthetic construct
  source 1..25: mol_type = other DNA
ctatgctgccaacatgcagtctttg

SEQ ID NO: 137 - DNA, 25 bp, synthetic construct
  source 1..25: mol_type = other DNA
catgtctgtgaattgctgagtacta

SEQ ID NO: 138 - DNA, 25 bp, synthetic construct
  source 1..25: mol_type = other DNA
tactaattgattcctccatccttga

SEQ ID NO: 139 - DNA, 25 bp, synthetic construct
  source 1..25: mol_type = other DNA
gaatcagttctcataatgcttttta

SEQ ID NO: 140 - DNA, 25 bp, synthetic construct
  source 1..25: mol_type = other DNA
atgctttttcctattaatactactt

SEQ ID NO: 141 - DNA, 25 bp, synthetic construct
  source 1..25: mol_type = other DNA
attgacatactgtgatctctattag

SEQ ID NO: 142 - DNA, 25 bp, synthetic construct
  source 1..25: mol_type = other DNA
tgttttcttacccttgacttacaat

SEQ ID NO: 143 - DNA, 25 bp, synthetic construct
  source 1..25: mol_type = other DNA
gtgaaattacttgtctgaaccccgt

SEQ ID NO: 144 - DNA, 25 bp, synthetic construct
  source 1..25: mol_type = other DNA
gacgactgtggggactatctggtga

SEQ ID NO: 145 - DNA, 25 bp, synthetic construct
  source 1..25: mol_type = other DNA
tgcggaaaacctcttcatccaattc

SEQ ID NO: 146 - DNA, 25 bp, synthetic construct
  source 1..25: mol_type = other DNA
cgccttcacctccaggtcaaagaag

SEQ ID NO: 147 - DNA, 25 bp, synthetic construct
  source 1..25: mol_type = other DNA
gttccaggtcccatacgtgacatat

SEQ ID NO: 148 - DNA, 25 bp, synthetic construct
  source 1..25: mol_type = other DNA
gcaggctctatgcatctgagaacca

SEQ ID NO: 149 - DNA, 25 bp, synthetic construct
  source 1..25: mol_type = other DNA
caaggctctgtttgatgtcctggcc

SEQ ID NO: 150 - DNA, 25 bp, synthetic construct
  source 1..25: mol_type = other DNA
gagtcccgagagatgtttccaagat

SEQ ID NO: 151 - DNA, 25 bp, synthetic construct
  source 1..25: mol_type = other DNA
tcgttcatccgattgctacgttcca

SEQ ID NO: 152 - DNA, 25 bp, synthetic construct
  source 1..25: mol_type = other DNA
tgctacgttccaaagtgtccaggtt

SEQ ID NO: 153 - DNA, 25 bp, synthetic construct
  source 1..25: mol_type = other DNA
accttacaaatgactcagcccacgt

SEQ ID NO: 154 - DNA, 25 bp, synthetic construct
  source 1..25: mol_type = other DNA
ttccttctgcatgtcagcacagtcg

SEQ ID NO: 155 - DNA, 25 bp, synthetic construct
  source 1..25: mol_type = other DNA
aggtggcagtggtccgtactccacc

SEQ ID NO: 156 - DNA, 25 bp, synthetic construct
  source 1..25: mol_type = other DNA
gtccaagatcggctccactgagaac

SEQ ID NO: 157 - DNA, 25 bp, synthetic construct
  source 1..25: mol_type = other DNA
actgagaacctgaagcaccagccgg

SEQ ID NO: 158 - DNA, 25 bp, synthetic construct
  source 1..25: mol_type = other DNA
gacctgagcaaggtgacctccaagt

SEQ ID NO: 159 - DNA, 25 bp, synthetic construct
  source 1..25: mol_type = other DNA
ggctcattaggcaacatccatcata

SEQ ID NO: 160 - DNA, 25 bp, synthetic construct
  source 1..25: mol_type = other DNA
gagtccagtcgaagattgggtccct

SEQ ID NO: 161 - DNA, 25 bp, synthetic construct
  source 1..25: mol_type = other DNA
tccctggacaatatcacccacgtcc

SEQ ID NO: 162 - DNA, 25 bp, synthetic construct
  source 1..25: mol_type = other DNA
cggagatcgtgtacaagtcgccagt

SEQ ID NO: 163 - DNA, 25 bp, synthetic construct
  source 1..25: mol_type = other DNA
agtcgccagtggtgtctggggacac

SEQ ID NO: 164 - DNA, 25 bp, synthetic construct
  source 1..25: mol_type = other DNA
gcagcatcgacatggtagactcgcc

SEQ ID NO: 165 - DNA, 25 bp, synthetic construct
  source 1..25: mol_type = other DNA
gctagctgacgaggtgtctgcctcc

SEQ ID NO: 166 - DNA, 25 bp, synthetic construct
  source 1..25: mol_type = other DNA
taatgtatctacatacctacaccta

SEQ ID NO: 167 - DNA, 25 bp, synthetic construct
  source 1..25: mol_type = other DNA
atctacatacctacacctatctata

SEQ ID NO: 168 - DNA, 25 bp, synthetic construct
  source 1..25: mol_type = other DNA
acacctatctatatataagctcatg

SEQ ID NO: 169 - DNA, 25 bp, synthetic construct
  source 1..25: mol_type = other DNA
gaaaaccatagctaagtagcatcgc

SEQ ID NO: 170 - DNA, 25 bp, synthetic construct
  source 1..25: mol_type = other DNA
gtagcatcgcagacttaagcgtaca

SEQ ID NO: 171 - DNA, 25 bp, synthetic construct
  source 1..25: mol_type = other DNA
aagcgtacaaagtgatcttgttcac

SEQ ID NO: 172 - DNA, 25 bp, synthetic construct
  source 1..25: mol_type = other DNA
tcttgttcacaagtaatctgttgac

SEQ ID NO: 173 - DNA, 25 bp, synthetic construct
  source 1..25: mol_type = other DNA
atctgttgacagtgccaataaatga

SEQ ID NO: 174 - DNA, 25 bp, synthetic construct
  source 1..25: mol_type = other DNA
catgtcacaatgtaacggatgacca

SEQ ID NO: 175 - DNA, 25 bp, synthetic construct
  source 1..25: mol_type = other DNA
cggatgaccatatgcacaattccat

SEQ ID NO: 176 - DNA, 25 bp, synthetic construct
  source 1..25: mol_type = other DNA
cctgtgttagtcagtattcttaaat

SEQ ID NO: 177 - DNA, 25 bp, synthetic construct
  source 1..25: mol_type = other DNA
aatgctctgcatggattggagacac

SEQ ID NO: 178 - DNA, 25 bp, synthetic construct
  source 1..25: mol_type = other DNA
ataactactgttgccatggaagggt

SEQ ID NO: 179 - DNA, 25 bp, synthetic construct
  source 1..25: mol_type = other DNA
aggagctggtttatcagtccgcttt

SEQ ID NO: 180 - DNA, 25 bp, synthetic construct
  source 1..25: mol_type = other DNA
atcagtccgctttgacatacagcta

SEQ ID NO: 181 - DNA, 25 bp, synthetic construct
  source 1..25: mol_type = other DNA
agtgtggacttatgccaaagggggc

SEQ ID NO: 182 - DNA, 25 bp, synthetic construct
  source 1..25: mol_type = other DNA
tgccaaagggggctgtttaagttga

SEQ ID NO: 183 - DNA, 25 bp, synthetic construct
  source 1..25: mol_type = other DNA
taagtttggcctcttgcctggaatc

SEQ ID NO: 184 - DNA, 25 bp, synthetic construct
  source 1..25: mol_type = other DNA
gaattctgaaatttcactgcgacgg

SEQ ID NO: 185 - DNA, 25 bp, synthetic construct
  source 1..25: mol_type = other DNA
ggtgcaagtctctctgcacctatta

SEQ ID NO: 186 - DNA, 25 bp, synthetic construct
  source 1..25: mol_type = other DNA
aaagtgatgtatatacttccttctt

SEQ ID NO: 187 - DNA, 25 bp, synthetic construct
  source 1..25: mol_type = other DNA
cttcttattctgttgagttgtatag

SEQ ID NO: 188 - DNA, 25 bp, synthetic construct
  source 1..25: mol_type = other DNA
attgctgcctctattatggcacttc

SEQ ID NO: 189 - DNA, 25 bp, synthetic construct
  source 1..25: mol_type = other DNA
ggcacttcaattttgcactgtcttt

SEQ ID NO: 190 - DNA, 25 bp, synthetic construct
  source 1..25: mol_type = other DNA
gtaaatgctgccatgttccaaaccc

SEQ ID NO: 191 - DNA, 25 bp, synthetic construct
  source 1..25: mol_type = other DNA
gtgtttagagctgtgcaccctagaa

SEQ ID NO: 192 - DNA, 25 bp, synthetic construct
  source 1..25: mol_type = other DNA
attatgccagtttctgttctctcac

SEQ ID NO: 193 - DNA, 25 bp, synthetic construct
  source 1..25: mol_type = other DNA
tttttgtgcactacatactcttcag

SEQ ID NO: 194 - DNA, 25 bp, synthetic construct
  source 1..25: mol_type = other DNA
gattaatatgcccttttgccgatgc

SEQ ID NO: 195 - DNA, 25 bp, synthetic construct
  source 1..25: mol_type = other DNA
tactgatgtgactcggttttgtcgc

SEQ ID NO: 196 - DNA, 25 bp, synthetic construct
  source 1..25: mol_type = other DNA
ttttgtcgcagctttgctttgttta

SEQ ID NO: 197 - DNA, 25 bp, synthetic construct
  source 1..25: mol_type = other DNA
cacacttgtaaacctcttttgcact

SEQ ID NO: 198 - DNA, 25 bp, synthetic construct
  source 1..25: mol_type = other DNA
gatgctcgagcacctgtaaacaatt

SEQ ID NO: 199 - DNA, 25 bp, synthetic construct
  source 1..25: mol_type = other DNA
gtcatatcatttcactgtctaggct

SEQ ID NO: 200 - DNA, 25 bp, synthetic construct
  source 1..25: mol_type = other DNA
gttgtcctttttatctgatctgtga

SEQ ID NO: 201 - DNA, 25 bp, synthetic construct
  source 1..25: mol_type = other DNA
aaatccacagctatatcctgatgct

SEQ ID NO: 202 - DNA, 25 bp, synthetic construct
  source 1..25: mol_type = other DNA
aatcttgtgtagtcttcaactggtt

SEQ ID NO: 203 - DNA, 25 bp, synthetic construct
  source 1..25: mol_type = other DNA
gtgtgaaatagttctgccacctctg

SEQ ID NO: 204 - DNA, 25 bp, synthetic construct
  source 1..25: mol_type = other DNA
atgctgtacgtactgcatttgcccc

SEQ ID NO: 205 - DNA, 25 bp, synthetic construct
  source 1..25: mol_type = other DNA
gagccaggtggatgtttaccgtgtg

SEQ ID NO: 206 - DNA, 25 bp, synthetic construct
  source 1..25: mol_type = other DNA
tgttatataacttcctggctccttc

SEQ ID NO: 207 - DNA, 25 bp, synthetic construct
  source 1..25: mol_type = other DNA
tccttcactgaacatgcctagtcca

SEQ ID NO: 208 - DNA, 25 bp, synthetic construct
  source 1..25: mol_type = other DNA
agtgagtcacatcctgggatccagt

SEQ ID NO: 209 - DNA, 25 bp, synthetic construct
  source 1..25: mol_type = other DNA
atgtcttgtgcataattcttccaaa

SEQ ID NO: 210 - DNA, 25 bp, synthetic construct
  source 1..25: mol_type = other DNA
tggaggtcaaactgggggagctgcc

SEQ ID NO: 211 - DNA, 25 bp, synthetic construct
  source 1..25: mol_type = other DNA
gggagctgccaagctggatcttgat

SEQ ID NO: 212 - DNA, 25 bp, synthetic construct
  source 1..25: mol_type = other DNA
ggatcttgatgcgggacttcagtcc

SEQ ID NO: 213 - DNA, 25 bp, synthetic construct
  source 1..25: mol_type = other DNA
ggacttcagtcctagtggcattttc

SEQ ID NO: 214 - DNA, 25 bp, synthetic construct
  source 1..25: mol_type = other DNA
tggcattttcggagcgtttcaaaga

SEQ ID NO: 215 - DNA, 25 bp, synthetic construct
  source 1..25: mol_type = other DNA
gcatctcggggattaccatggtgct

SEQ ID NO: 216 - DNA, 25 bp, synthetic construct
  source 1..25: mol_type = other DNA
accatggtgctggcatgctacgtgc

SEQ ID NO: 217 - DNA, 25 bp, synthetic construct
  source 1..25: mol_type = other DNA
atgctacgtgctctttagctactcc

SEQ ID NO: 218 - DNA, 25 bp, synthetic construct
  source 1..25: mol_type = other DNA
tccttttcctacaagcatctcaagc

SEQ ID NO: 219 - DNA, 25 bp, synthetic construct
  source 1..25: mol_type = other DNA
agcccctccgtgaggaacacaatct

SEQ ID NO: 220 - DNA, 25 bp, synthetic construct
  source 1..25: mol_type = other DNA
atctcaatcgttgctgaatcctttc

SEQ ID NO: 221 - DNA, 25 bp, synthetic construct
  source 1..25: mol_type = other DNA
gacatcaggtaccaactgcactcgt

SEQ ID NO: 222 - DNA, 25 bp, synthetic construct
  source 1..25: mol_type = other DNA
tgcactcgttttggcattgcagcta

SEQ ID NO: 223 - DNA, 25 bp, synthetic construct
  source 1..25: mol_type = other DNA
atatcagttggatcccactgcttcc

SEQ ID NO: 224 - DNA, 25 bp, synthetic construct
  source 1..25: mol_type = other DNA
ctcagactctgaggcctggtgtgaa

SEQ ID NO: 225 - DNA, 25 bp, synthetic construct
  source 1..25: mol_type = other DNA
tggtgtgaagcttacactctctgct

SEQ ID NO: 226 - DNA, 25 bp, synthetic construct
  source 1..25: mol_type = other DNA
actctctgctctggtagatgggaag

SEQ ID NO: 227 - DNA, 25 bp, synthetic construct
  source 1..25: mol_type = other DNA
atatatttccattgtgaccagcagc

SEQ ID NO: 228 - DNA, 25 bp, synthetic construct
  source 1..25: mol_type = other DNA
tgtgaccagcagcaggcttttttcc

SEQ ID NO: 229 - DNA, 25 bp, synthetic construct
  source 1..25: mol_type = other DNA
tagttaccaatgctgcagtcctgca

SEQ ID NO: 230 - DNA, 25 bp, synthetic construct
  source 1..25: mol_type = other DNA
gtcctgcagtcacctatacattatt

SEQ ID NO: 231 - DNA, 25 bp, synthetic construct
  source 1..25: mol_type = other DNA
ttaactgttaaatgcgctacccacc

SEQ ID NO: 232 - DNA, 25 bp, synthetic construct
  source 1..25: mol_type = other DNA
cagttcccatgtgagccattcaaat

SEQ ID NO: 233 - DNA, 25 bp, synthetic construct
  source 1..25: mol_type = other DNA
gacgatctgagcacaccaaatgaaa

SEQ ID NO: 234 - DNA, 25 bp, synthetic construct
  source 1..25: mol_type = other DNA
atatccattggctgtaagacctttc

SEQ ID NO: 235 - DNA, 25 bp, synthetic construct
  source 1..25: mol_type = other DNA
taagacctttctataccatgcctga

SEQ ID NO: 236 - DNA, 25 bp, synthetic construct
  source 1..25: mol_type = other DNA
tgcctgacccaagaaatcccaaaca

SEQ ID NO: 237 - DNA, 25 bp, synthetic construct
  source 1..25: mol_type = other DNA
gaatacatgatcctcaactgctaac

SEQ ID NO: 238 - DNA, 25 bp, synthetic construct
  source 1..25: mol_type = other DNA
ttacattgattccttccgctttgga

SEQ ID NO: 239 - DNA, 25 bp, synthetic construct
  source 1..25: mol_type = other DNA
acgagttactatgctgtttctggga

SEQ ID NO: 240 - DNA, 25 bp, synthetic construct
  source 1..25: mol_type = other DNA
taatgttcgtcagacctccatgttc

SEQ ID NO: 241 - DNA, 25 bp, synthetic construct
  source 1..25: mol_type = other DNA
cgactcactccttaaattcacactt

SEQ ID NO: 242 - DNA, 25 bp, synthetic construct
  source 1..25: mol_type = other DNA
cacactttgccacttaactccagtg

SEQ ID NO: 243 - DNA, 25 bp, synthetic construct
  source 1..25: mol_type = other DNA
ttaatgtgccaaggagccgttttct

SEQ ID NO: 244 - DNA, 25 bp, synthetic construct
  source 1..25: mol_type = other DNA
ccgttttctgcctgtcaaaaccaca

SEQ ID NO: 245 - DNA, 25 bp, synthetic construct
  source 1..25: mol_type = other DNA
aaaaccacatcagatctcttgctgg

SEQ ID NO: 246 - DNA, 25 bp, synthetic construct
  source 1..25: mol_type = other DNA
gtgatgtcaaacctctatagtctta

SEQ ID NO: 247 - DNA, 25 bp, synthetic construct
  source 1..25: mol_type = other DNA
tttcctacagtgcccttggttaaat

SEQ ID NO: 248 - DNA, 25 bp, synthetic construct
  source 1..25: mol_type = other DNA
taggcagttcttttacgaaggttca

SEQ ID NO: 249 - DNA, 25 bp, synthetic construct
  source 1..25: mol_type = other DNA
gaattggatcacctcacagtttcag

SEQ ID NO: 250 - DNA, 25 bp, synthetic construct
  source 1..25: mol_type = other DNA
aagggaacggttatcatcattgcaa

SEQ ID NO: 251 - DNA, 25 bp, synthetic construct
  source 1..25: mol_type = other DNA
agaattgatatcccacctggagcag

SEQ ID NO: 252 - DNA, 25 bp, synthetic construct
  source 1..25: mol_type = other DNA
gtctggaaaccttcgcatcttggac

SEQ ID NO: 253 - DNA, 25 bp, synthetic construct
  source 1..25: mol_type = other DNA
actttactatgttactgtaccctgc

SEQ ID NO: 254 - DNA, 25 bp, synthetic construct
  source 1..25: mol_type = other DNA
tccccgtacttccagatttattgtt

SEQ ID NO: 255 - DNA, 25 bp, synthetic construct
  source 1..25: mol_type = other DNA
gtcagtgtccagccatgcataaggg

SEQ ID NO: 256 - DNA, 25 bp, synthetic construct
  source 1..25: mol_type = other DNA
agaggatagtgtgtacctgccctgc

SEQ ID NO: 257 - DNA, 25 bp, synthetic construct
  source 1..25: mol_type = other DNA
tgccctctgctatgaaggtctctgc

SEQ ID NO: 258 - DNA, 25 bp, synthetic construct
  source 1..25: mol_type = other DNA
tgccttgtggatcatgggactcccc

SEQ ID NO: 259 - DNA, 25 bp, synthetic construct
  source 1..25: mol_type = other DNA
ggactccccttggaggatctgtgca

SEQ ID NO: 260 - DNA, 25 bp, synthetic construct
  source 1..25: mol_type = other DNA
ttggtctgtgggtccagtgttgtct

SEQ ID NO: 261 - DNA, 25 bp, synthetic construct
  source 1..25: mol_type = other DNA
gtctgtcatccatttaagtgttccc

SEQ ID NO: 262 - DNA, 25 bp, synthetic construct
  source 1..25: mol_type = other DNA
ttcaagtgacaatcctctccttggc

SEQ ID NO: 263 - DNA, 25 bp, synthetic construct
  source 1..25: mol_type = other DNA
agagcatgtctggcatagcagcctg

SEQ ID NO: 264 - DNA, 25 bp, synthetic construct
  source 1..25: mol_type = other DNA
tagcagcctgacttttatgccctaa

SEQ ID NO: 265 - DNA, 25 bp, synthetic construct
  source 1..25: mol_type = other DNA
aagccagtgctctaagacctcagct

SEQ ID NO: 266 - DNA, 25 bp, synthetic construct
  source 1..25: mol_type = other DNA
cttttatctcagaaccccatgggtt

SEQ ID NO: 267 - DNA, 25 bp, synthetic construct
  source 1..25: mol_type = other DNA
tcaaattgttgtcctgtctgtctat

SEQ ID NO: 268 - DNA, 25 bp, synthetic construct
  source 1..25: mol_type = other DNA
gagctttgattactgactccggttc

SEQ ID NO: 269 - DNA, 25 bp, synthetic construct
  source 1..25: mol_type = other DNA
ccctgacttaccactaatttactag

SEQ ID NO: 270 - DNA, 25 bp, synthetic construct
  source 1..25: mol_type = other DNA
tcatgagtaacctctcacagctacc

SEQ ID NO: 271 - DNA, 25 bp, synthetic construct
  source 1..25: mol_type = other DNA
ccttcttttatctgcactgtgtgaa

SEQ ID NO: 272 - DNA, 25 bp, synthetic construct
  source 1..25: mol_type = other DNA
gcaagtgtcctaagctatgtcatcc

SEQ ID NO: 273 - DNA, 25 bp, synthetic construct
  source 1..25: mol_type = other DNA
gtcatccaaagattgtcctttccat

SEQ ID NO: 274 - DNA, 25 bp, synthetic construct
  source 1..25: mol_type = other DNA
ttccattctcaaatcctgtgactgg

SEQ ID NO: 275 - DNA, 25 bp, synthetic construct
  source 1..25: mol_type = other DNA
gactgggatcactcaacagcactgt

SEQ ID NO: 276 - DNA, 25 bp, synthetic construct
  source 1..25: mol_type = other DNA
acttttaagtcctgcctgtactgaa

SEQ ID NO: 277 - DNA, 25 bp, synthetic construct
  source 1..25: mol_type = other DNA
ctgtactgaagttcacagcccacct

SEQ ID NO: 278 - DNA, 25 bp, synthetic construct
  source 1..25: mol_type = other DNA
ttcccaggactgcacggtttaacac

SEQ ID NO: 279 - DNA, 25 bp, synthetic construct
  source 1..25: mol_type = other DNA
gatattcagatactcttctatgcca

SEQ ID NO: 280 - DNA, 25 bp, synthetic construct
  source 1..25: mol_type = other DNA
ttcagtagatcttccttggcagcca

SEQ ID NO: 281 - DNA, 25 bp, synthetic construct
  source 1..25: mol_type = other DNA
ggcagccagccataggttgtttctt

SEQ ID NO: 282 - DNA, 25 bp, synthetic construct
  source 1..25: mol_type = other DNA
ttgtcttccgggtcctaaagagcac

SEQ ID NO: 283 - DNA, 25 bp, synthetic construct
  source 1..25: mol_type = other DNA
ggaggtccccagtctaggtaggaag

SEQ ID NO: 284 - DNA, 25 bp, synthetic construct
  source 1..25: mol_type = other DNA
gacttctttttttccgacagcagga

SEQ ID NO: 285 - DNA, 25 bp, synthetic construct
  source 1..25: mol_type = other DNA
gctccacacaccagatgctttggtt

SEQ ID NO: 286 - DNA, 25 bp, synthetic construct
  source 1..25: mol_type = other DNA
gcaaggagaccatggctactctttg

SEQ ID NO: 287 - DNA, 25 bp, synthetic construct
  source 1..25: mol_type = other DNA
atcgaggtctcattaaactttacaa

SEQ ID NO: 288 - DNA, 25 bp, synthetic construct
  source 1..25: mol_type = other DNA
ggaaccggttgcgagatcggcaata

SEQ ID NO: 289 - DNA, 25 bp, synthetic construct
  source 1..25: mol_type = other DNA
ctcttgaagatgtcagcaccagccc

SEQ ID NO: 290 - DNA, 25 bp, synthetic construct
  source 1..25: mol_type = other DNA
agaagtggtgcagtccttcctgatt

SEQ ID NO: 291 - DNA, 25 bp, synthetic construct
  source 1..25: mol_type = other DNA
tcagaatgtcgagggaccgagctgc

SEQ ID NO: 292 - DNA, 25 bp, synthetic construct
  source 1..25: mol_type = other DNA
gaggacctcagttaaccaattacct

SEQ ID NO: 293 - DNA, 25 bp, synthetic construct
  source 1..25: mol_type = other DNA
tttgcattgaaacactggtccagtc

SEQ ID NO: 294 - DNA, 25 bp, synthetic construct
  source 1..25: mol_type = other DNA
gaagaccctattttgggtgatccct

SEQ ID NO: 295 - DNA, 25 bp, synthetic construct
  source 1..25: mol_type = other DNA
ggtgatccctatcttgcagaatgtc

SEQ ID NO: 296 - DNA, 25 bp, synthetic construct
  source 1..25: mol_type = other DNA
accgcatgtgtaagtagtctggccc

SEQ ID NO: 297 - DNA, 25 bp, synthetic construct
  source 1..25: mol_type = other DNA
tggcccacattttcaacctagtaga

SEQ ID NO: 298 - DNA, 25 bp, synthetic construct
  source 1..25: mol_type = other DNA
gttgttccaatgaatgtacagcact

SEQ ID NO: 299 - DNA, 25 bp, synthetic construct
  source 1..25: mol_type = other DNA
tgtacagcacttccattaacttttg

SEQ ID NO: 300 - DNA, 25 bp, synthetic construct
  source 1..25: mol_type = other DNA
acatgggaatgttctgtcatcaatg

SEQ ID NO: 301 - DNA, 25 bp, synthetic construct
  source 1..25: mol_type = other DNA
ctgtcatcaatggagtgtattcttg

SEQ ID NO: 302 - DNA, 25 bp, synthetic construct
  source 1..25: mol_type = other DNA
agaattctttatatcgttctcaatt

SEQ ID NO: 303 - DNA, 25 bp, synthetic construct
  source 1..25: mol_type = other DNA
atatcgttctcaattctatagactt

SEQ ID NO: 304 - DNA, 25 bp, synthetic construct
  source 1..25: mol_type = other DNA
ctatagactttcaagcctatgtatg

SEQ ID NO: 305 - DNA, 25 bp, synthetic construct
  source 1..25: mol_type = other DNA
gtacattccatctttataggtctgt

SEQ ID NO: 306 - DNA, 25 bp, synthetic construct
  source 1..25: mol_type = other DNA
tttataggtctgtttcatatgtttt

SEQ ID NO: 307 - DNA, 25 bp, synthetic construct
  source 1..25: mol_type = other DNA
agaacactaagtcttgcactctctg

SEQ ID NO: 308 - DNA, 25 bp, synthetic construct
  source 1..25: mol_type = other DNA
tgcactctctgacattgatactgat

SEQ ID NO: 309 - DNA, 25 bp, synthetic construct
  source 1..25: mol_type = other DNA
gcaccttcaaagggacacctacggc

SEQ ID NO: 310 - DNA, 25 bp, synthetic construct
  source 1..25: mol_type = other DNA
agtacctgggtctggacgtgccagt

SEQ ID NO: 311 - DNA, 25 bp, synthetic construct
  source 1..25: mol_type = other DNA
gccatgccaggaacctgtcctaagg

SEQ ID NO: 312 - DNA, 25 bp, synthetic construct
  source 1..25: mol_type = other DNA
cctgcttgagttcccagatggctgg

SEQ ID NO: 313 - DNA, 25 bp, synthetic construct
  source 1..25: mol_type = other DNA
gagtctttgtggattctgaggccct

SEQ ID NO: 314 - DNA, 25 bp, synthetic construct
  source 1..25: mol_type = other DNA
ggccctgcccaatgagactctaggg

SEQ ID NO: 315 - DNA, 25 bp, synthetic construct
  source 1..25: mol_type = other DNA
gactctagggtccagtggatgccac

SEQ ID NO: 316 - DNA, 25 bp, synthetic construct
  source 1..25: mol_type = other DNA
ctttccttccagatcctgggtactg

SEQ ID NO: 317 - DNA, 25 bp, synthetic construct
  source 1..25: mol_type = other DNA
aagcgacccattcagagactgtccc

SEQ ID NO: 318 - DNA, 25 bp, synthetic construct
  source 1..25: mol_type = other DNA
agactgtccctgaaacctagtactg

SEQ ID NO: 319 - DNA, 25 bp, synthetic construct
  source 1..25: mol_type = other DNA
ggtgtcagtatccaggctttgtaca

SEQ ID NO: 320 - DNA, 25 bp, synthetic construct
  source 1..25: mol_type = other DNA
ccctcaatctagaacgctacacaag

SEQ ID NO: 321 - DNA, 25 bp, synthetic construct
  source 1..25: mol_type = other DNA
aaataggaacacgtgctctacctcc

SEQ ID NO: 322 - DNA, 25 bp, synthetic construct
  source 1..25: mol_type = other DNA
gtgctctacctccatttagggattt

SEQ ID NO: 323 - DNA, 25 bp, synthetic construct
  source 1..25: mol_type = other DNA
ctacctccatttagggatttgcttg

SEQ ID NO: 324 - DNA, 25 bp, synthetic construct
  source 1..25: mol_type = other DNA
ttagggatttgcttgggatacagaa

SEQ ID NO: 325 - DNA, 25 bp, synthetic construct
  source 1..25: mol_type = other DNA
gggatacagaagaggccatgtgtct

SEQ ID NO: 326 - DNA, 25 bp, synthetic construct
  source 1..25: mol_type = other DNA
gaagaggccatgtgtctcagagctg

SEQ ID NO: 327 - DNA, 25 bp, synthetic construct
  source 1..25: mol_type = other DNA
gaggccatgtgtctcagagctgtta

SEQ ID NO: 328 - DNA, 25 bp, synthetic construct
  source 1..25: mol_type = other DNA
gtgtctcagagctgttaagggctta

SEQ ID NO: 329 - DNA, 25 bp, synthetic construct
  source 1..25: mol_type = other DNA
cagagctgttaagggcttatttttt

SEQ ID NO: 330 - DNA, 25 bp, synthetic construct
  source 1..25: mol_type = other DNA
cattggagtcatagcatgtgtgtaa

SEQ ID NO: 331 - DNA, 25 bp, synthetic construct
  source 1..25: mol_type = other DNA
gatggagatcctacaaacacgtcag

SEQ ID NO: 332 - DNA, 25 bp, synthetic construct
  source 1..25: mol_type = other DNA
aacacgtcagtgggcagatgctgta

SEQ ID NO: 333 - DNA, 25 bp, synthetic construct
  source 1..25: mol_type = other DNA
attctattcattatgccttaccatg

SEQ ID NO: 334 - DNA, 25 bp, synthetic construct
  source 1..25: mol_type = other DNA
tgccttaccatgtggcagatcccac

SEQ ID NO: 335 - DNA, 25 bp, synthetic construct
  source 1..25: mol_type = other DNA
ggcagatcccacaggagtttgtcaa

SEQ ID NO: 336 - DNA, 25 bp, synthetic construct
  source 1..25: mol_type = other DNA
aggagttgtgtcgagctcacagcgt

SEQ ID NO: 337 - DNA, 25 bp, synthetic construct
  source 1..25: mol_type = other DNA
gctcacagcgtttctatcaacttac

SEQ ID NO: 338 - DNA, 25 bp, synthetic construct
  source 1..25: mol_type = other DNA
acaacttcatctgtactgcttgaat

SEQ ID NO: 339 - DNA, 25 bp, synthetic construct
  source 1..25: mol_type = other DNA
ccagtcccgggcactgagtgttgaa

SEQ ID NO: 340 - DNA, 25 bp, synthetic construct
  source 1..25: mol_type = other DNA
atgatgtctgaagttattgctgcac

SEQ ID NO: 341 - DNA, 25 bp, synthetic construct
  source 1..25: mol_type = other DNA
attgctgcacaattacccaagatat

SEQ ID NO: 342 - DNA, 5525 bp, Homo sapiens
  source 1..5525: mol_type = other DNA
ggggacggtgggagcagatccatttccgggttggcaaaaggggcggtggcggcggcggcggcggcggcggcggcggcgacggcgagaaagagcttgccggggggcgagcaggacaggacgaagccggagtgtaggcggcagaggattcgctcccagagcagctgcggccaggtatatgaatgacctaaaggtacaaataaagacggagagagaacagtgccaactgggagcagggcaagaatgccaattcctcctcccccgccacccccacctggtcctcctccacctcccacatttcatcaggcaaacacagagcagcccaagctgagtagagatgagcagcggggtcgaggcgccctcttacaggacatttgcaaagggaccaagctgaagaaggtgaccaacattaatgatcggagtgctcccatcctcgagaagccgaaaggaagcagtggtggctatggctctggaggagctgccctgcagcccaagggaggtctcttccaaggaggagtgctgaagcttcgacctgtgggagccaaggatggttcagagaacctagctggtaagccagccctgcaaatccccagttctcgagctgctgccccaaggcctccagtatctgccgccagcgggcgtcctcaggatgatacagacagcagccgggcctcactcccagaactgccccggatgcagagaccctctttaccggacctctctcggcctaataccaccagcagtacgggcatgaagcacagctcctctgcccctcccccaccacccccagggcggcgtgccaacgcaccccccacacctctgcctatgcacagcagcaaagcccccgcctacaacagagagaaacccttgccaccgacgcctggacaaaggcttcaccctggtcgagagggacctcctgctccacccccagtcaaaccacctccttcccctgtgaatatcagaacaggaccaagtggccagtctctggctcctcctcctccgccttaccgccagcctcctggggtccccaatggaccctctagccccactaatgagtcagcccctgagctgccacagagacacaattctttgcataggaagacaccagggcctgtcagaggcctagcacctcctccacccacctcggcctccccatctttactgagtaataggccacctcccccagcccgagaccctcccagtcggggagcagctcctccacccccaccacctgtgatccgaaatggtgccagggatgctccccctcccccaccaccataccgaatgcatgggtcagaacccccgagccgaggaaagcccccacctccaccctcaaggacgccagctgggccaccccctcctcctccaccgcccctgaggaatggccacagagattctatcaccactgtccggtctttcttggatgattttgagtcaaagtattccttccatccagtagaagactttcctgctccagaagaatataaacactttcagaggatatatcccagcaaaacaaaccgagctgcccgtggagccccacctctgccacccattctcaggtgaagcctggcttggtcccgttcctcaggaaaaggatggaccttctcttcttctcagatggtcccttccattcccctgaaacctgcatgagagctcctaacatgtttctccaatgcaatcaagccctagactccaaatgtcctcccagctcacctccatctatgcatctcatctctggatttggtgatcagactctatattgacagtaggatctcaaaccctgcatccatccttcctccagcaagccctgctagccacatgaggaacaagtttccgtgtcttctgccttcctcttggggaaaggtgccttgttgtgatgaattaactcactgttagggcagggtggagaatggtactccttccttctcctgtccactgtgggggaagcttggcaggtatattatatttcatcatttaggaggctggcatgaccaggacttatgggtgggaggggagcatttttagtgaagcaagaaaggagtttgccaagaagtgatctgttttaaaggtcatatttggagaaagggcaaggaattgggtctgctttatttttgggggtattttgtttttgttctcacctgctgcccccccaccccaccaccccagggataaattggatataaacactaaatactaatcagttgaacttaacatttaataaaaagaaagggtgaaataaactgaagaccattttagaactagtcagttctctgcagcaaagggaacaggagccatttgaaccctctgggacccctcaccccactgcttcagggtgctaggctgagggatgtttttcctcccccttaccgcccatgcccttgaaagaaaagtcactttttgtggagggcatcattcattcctgattcacaaaccccaaaaacctctggtgggagataggaagatagggcgtgggcctgggccttaacctcaatcttgtgtctgcctcagtcttttctgactggccctgaagttgtcagtggctctttctgtccttcagcccctggaaggtgctccaggataacaaagaagggcaggttgaagcccctcatggaaggagctggctttgtggggctgcaaaggacttttaagtcctgcctgtactgaagttcacagcccacctgactgagcagactcttcctgttcctttctctaccacccttgccttcccaggactgcacggtttaacacagcagagtacagaagggtgaagaagtgagcagaggcttatgaagatattcagatactcttctatgccaggaagcacaaagactttgttgagatttgcctcagttcagtagatcttccttggcagccagccataggttgtttctttgtcttccgggtcctaaagagcacagagaaaatggaggtccccagtctaggtaggaagctgattggatgaggacttctttttttccgacagcaggatggggctcttgggctccacacaccagatgctttggttttctacaactgttgctatgtgtagagggtgctcagagcgtggcatgagagcaaggagaccatggctactctttgaaatggatggggaaaattagcttaaaaatttaatcacgagattgcgccactgcactccagcctgggcgacagagccagactccgtctcaaaaaaaaaaaaaaaaaaaaaatttaatcacacacatcacaggcttaaactgtcttagagacctttccagttgattggatcacaggctagggatctgtgcatggtgccaggtgaaacccaagtctccccagtgctaggtgagggagggctcaacgggggtaatgtgtatggggtctgctgtcctttgccatcccctggaccatattaactctgggacagaccatgattaggtctgtcaccaaacttttcctaatcattttttatgtatcattcttctttctaatctgttagcagttatctcccagaagtatccctagcatcctgaagcaaacagaactgccgggcgctgtggcttatgcctgtaatctcagcaatttgggaggccgaggcgggcggatcacctgaggtcagaagttcgagaccagcctgtccaacatggtgaaaccccgtctccactaaaaacacaaaaattatctgggcgtggtggcatgtgcctgtagtcccagctactggggaggctgaggcaggagaatcgcttgaatccaggaggtggaggttgcagtgagtggagatggcgccactgcgctccagcccgggtgacagagtgaaactccatctcagaaaacaaacaaaccaaacaactgagagagggcatgctagtggagggaaaaggagagagtacaagagttgatactgcatcagagttccagggcagatactgacaagtgactgaactttgactgtgaagtcttcccatttatttttgaaatgggagtcgatgccttttgtacgttatcaaagtgtttttttttttttcctcctttactcagacatagagcatcaaaatatcacgagtaaagccaaaccccagcttttcactggggctgatatcttcctccccatggcccactgccccctcatattccccagcacttggactgtgtcacatgggtattgattgtatacttgttgtcttggcctcatgcaaaaaggcctgggtctagatctagtcagatgagttttttttagagcatgttgatgatacaacacctgtttaaatgtctgccttatatgttatttggtcccactgttaataatagatttattactatgtatatttactattgaagatgggaatgtgatctgcatagttatcagtctattttgtatgttgtaaaaagcttctaatttaggtttaaggtgggctggctacaagagcactaaaactcctcaccttttaaactgctctttttatctgcttgtgggaatgtcgtctctttcgtggaagattgggtggtctcatgttgaggctgttgcccagtcccattaactcccttgtccccccacagaaggaagagacattgcccagctaagcatcaggaagctgtgttaaaagcccttctatgggtttggttttgtgatgtttttccctaatgggaaaaacgttatagttgtttcttactgccctgtctgggaagcagggcaaacctccaggtttttaaatgagctagatgcccctcttcctcttctctggtcactgaacctggaccaaagcactttgatattccaggtgtgattttctctgtcatggggatttgctccactgcagagccccatcattttcacagcgtaggccaacagagtgagaacctagggctaccctagctgatggatgtgaggctgctgtctacaggagctcatcccagccctgttaactggcagtggcaaggatactcgtcatcggccattgcactggggaactccctcaccccatggcttcccaacttgaaacccagatttacctccagggagaggtgagaaaaaaattgtaaatagacttgctaaagagcaactcagggttggggtgtgttttaattctcctgatcacttgaaataatctgtaggctgagtgcttatgggggtgggggagaagggtgactccagggtccttccattttgtgaagctctgggggtggagtgtgggcatctgaggccctatgatggcactacattgagctgtctgcccttccggaaacccaacctgcaatcaactgcaaatcaaattcttcacattccagctacagtctttctttccccattgaatctcagtccctggccatgtggtcaaggtggctttctgttaagctaccctaatttcgggaatgggaggggagagaggagggccattacaactctgccttcaagactcatctcttaaaaacaaaacgaaacaaaactacaaccaccatcaaaaccacacgcaaaaaaaaaaaaaaaaaaaaaaaggataactttaaccgaaggaagggtttggttccattcaactccacattcattg

SEQ ID NO: 343 - DNA, 4209 bp, Homo sapiens
  source 1..4209: mol_type = other DNA
cttctggatgtgagacaggcggatctcacccacgcctgccagcagctcctcggagaactgtgttcctgggtcagccctggcccagaggagcgccggggacccgcagagtgctgctgaagtcaaggctacaactcacctaggatctggggcgccagccctccggtgggcagggcgttctcctcccccaccccctccccgcacgatgacatcaagtgtttggcgttgagttgctccataaaagctgcccggggaagccaggagagcgaagggcggacgtactcgccacggcacccaggctgcgcgcacgcggtcccggtgtgcagctggagagcgagcggccaccgggagcccccggcacagcccgcgcccgccccgcaggagcccgcgaagatgccccggcgcagcctgcacgcggcggccgtgctcctgctggtgatcttaaaggaacagccttccagcccggccccagtgaacggttccaagtggacttattttggtcctgatggggagaatagctggtccaagaagtacccgtcgtgtgggggcctgctgcagtcccccatagacctgcacagtgacatcctccagtatgacgccagcctcacgcccctcgagttccaaggctacaatctgtctgccaacaagcagtttctcctgaccaacaatggccattcagtgaagctgaacctgccctcggacatgcacatccagggcctccagtctcgctacagtgccacgcagctgcacctgcactgggggaacccgaatgacccgcacggctctgagcacaccgtcagcggacagcacttcgccgccgagctgcacattgtccattataactcagacctttatcctgacgccagcactgccagcaacaagtcagaaggcctcgctgtcctggctgttctcattgagatgggctccttcaatccgtcctatgacaagatcttcagtcaccttcaacatgtaaagtacaaaggccaggaagcattcgtcccgggattcaacattgaagagctgcttccggagaggaccgctgaatattaccgctaccgggggtccctgaccacacccccttgcaaccccactgtgctctggacagttttccgaaaccccgtgcaaatttcccaggagcagctgctggctttggagacagccctgtactgcacacacatggacgacccttcccccagagaaatgatcaacaacttccggcaggtccagaagttcgatgagaggctggtatacacctccttctcccaagtgcaagtctgtactgcggcaggactgagtctgggcatcatcctctcactggccctggctggcattcttggcatctgtattgtggtggtggtgtccatttggcttttcagaaggaagagtatcaaaaaaggtgataacaagggagtcatttacaagccagccaccaagatggagactgaggcccacgcttgaggtccccggagctcccgggcacatccaggaaggaccttgctttggaccctacacacttcggctctctggacacttgcgacacctcaaggtgttctctgtagctcaatctgcaaacatgccaggcctcagggatcctctgctgggtgcctccttgccttgggaccatggccaccccagagccatccgatcgatggatgggatgcactctcagaccaagcagcaggaattcaaagctgcttgctgtaactgtgtgagattgtgaagtggtctgaattctggaatcacaaaccaagccatgctggtgggccattaatggttggaaaacactttcatccggggctttgccagagcgtgctttcaagtgtcctggaaattctgctgcttctccaagctttcagacaagaatgtgcactctctgcttaggttttgcttgggaaactcaacttctttcctctggagacggggcatctccctctgatttccttctgctatgacaaaacctttaatctgcaccttacaactcggggacaaatggggacaggaaggatcaagttgtagagagaaaaagaaaacaagagatatacattgtgatatattagggacactttcacagtcctgtcctctggatcacagacactgcacagaccttagggaatggcaggttcaagttccacttcttggtggggatgagaagggagagagagctagagggacaaagagaatgagaagacatggatgatctgggagagtctcactttggaatcagaattggaatcacattctgtttatcaagccataatgtaaggacagaataatacaatattaagtccaaatccaacctcctgtcagtggagcagttatgttttatactctacagattttacaaataatgaggctgttccttgaaaatgtgttgttgctgtgtcctggaggagacatgagttccgagatgacccaatctgcctttgaatctggaggaaataggcagaaacaaaatgactgtagaacttattctctgtaggccaaatttcatttcagccacttctgcaggatccctactgccaacctggaatggagacttttatctacttctctctctctgaagatgtcaaatcgtggtttagatcaaatatatttcaagctataaaagcaggaggttatctgtgcagggggctggcatcatgtatttaggggcaagtaataatggaatgctactaagatactccatattcttccccgaatcacacagacagtttctgacaggcgcaactcctccattttcctcccgcaggtgagaaccctgtggagatgagtcagtgccatgactgagaaggaaccgacccctagttgagagcaccttgcagttccccgagaactttctgattcacagtctcattttgacagcatgaaatgtcctcttgaagcatagctttttaaatatctttttccttctactcctccctctgactctaagaattctctcttctggaatcgcttgaacccaggaggcggaggttgcagtaagccaaggtcatgccactgcactctagcctgggtgacagagcgagactccatctcaaaaaaaaaaaaaaaaaaattattctgtaccatcacaacttttcacaacgatggcaagccttatgtcttgggagcctgttttgctaggcaaagttacaagtgacctaatgggagctcaaatgtgtgtgtgtctctctgtgtgtttgtgtgtgtgtgtgcactcaagacctctaacagcctcgaagcctggggtggcatcccggccttgccattagcatgcctcatgcatcatcagatgacaaggacaaccctcatgacgaagcaacatgaattagggggcctcttggccttggtccaaaattgtcaatcagaaatgaacataaaggactccagagcagtgggactgtctgtcaaaagactctgtatatcttttgtggatgagttttgtgagagaacagagagaccattgtacctggcacaagggctgttcatgaaaagggagacttactgggaggtgcaagacagtggcatttctcctctcctcttgctgctcagcacagccctggattgcagccccgaggctgagaccagacaaagcccgggaggcagaaagatgctccaagaaccaacactatcaatgtctttgcaaatcctcacaggattcctgtgggtccagctttggaactgggaaacctttcttcggatccgcactcattccactgatgccagctgcccctgaaggatgccagtactgtggtgtgtgagtctcagcagccgcccacacgctcctaactctgctgcatggcagatgcctaggtggaaatagcaaaaacaaggcccaggctggggccagggccagaggggaaggccctggattctcactcatgtgagatcttgaatctctttctttgttctgtttgtttagttagtatcatctggtaaaatagttaaaaaacaacaaaaaactctgtatctgtttctagcatgtgctgcattgactctattaatcacatttcaaattcaccctacattcctctcctcttcactagcctctctgaaggtgtcctggccagccctggagaagcactggtgtctgcagcacccctcagttcctgtgcctcagcccacaggccactgtgataatggtctgtttagcacttctgtatttattgtaagaatgattataatgaagatacacactgtaactacaagaaattataaatgtttttcacatca

SEQ ID NO: 344 - DNA, 4550 bp, Homo sapiens
  source 1..4550: mol_type = other DNA
ccgcgcacacctccccgcgccgccgccgccaccgcccgcactccgccgcctctgcccgcaaccgctgagccatccatgggggtcgcgggccgcaaccgtcccggggcggcctgggcggtgctgctgctgctgctgctgctgccgccactgctgctgctggcgggggccgtcccgccgggtcggggccgtgccgcggggccgcaggaggatgtagatgagtgtgcccaagggctagatgactgccatgccgacgccctgtgtcagaacacacccacctcctacaagtgctcctgcaagcctggctaccaaggggaaggcaggcagtgtgaggacatcgatgaatgtggaaatgagctcaatggaggctgtgtccatgactgtttgaatattccaggcaattatcgttgcacttgttttgatggcttcatgttggctcatgacggtcataattgtcttgatgtggacgagtgcctggagaacaatggcggctgccagcatacctgtgtcaacgtcatggggagctatgagtgctgctgcaaggaggggtttttcctgagtgacaatcagcacacctgcattcaccgctcggaagagggcctgagctgcatgaataaggatcacggctgtagtcacatctgcaaggaggccccaaggggcagcgtcgcctgtgagtgcaggcctggttttgagctggccaagaaccagagagactgcatcttgacctgtaaccatgggaacggtgggtgccagcactcctgtgacgatacagccgatggcccagagtgcagctgccatccacagtacaagatgcacacagatgggaggagctgccttgagcgagaggacactgtcctggaggtgacagagagcaacaccacatcagtggtggatggggataaacgggtgaaacggcggctgctcatggaaacgtgtgctgtcaacaatggaggctgtgaccgcacctgtaaggatacttcgacaggtgtccactgcagttgtcctgttggattcactctccagttggatgggaagacatgtaaagatattgatgagtgccagacccgcaatggaggttgtgatcatttctgcaaaaacatcgtgggcagttttgactgcggctgcaagaaaggatttaaattattaacagatgagaagtcttgccaagatgtggatgagtgctctttggataggacctgtgaccacagctgcatcaaccaccctggcacatttgcttgtgcttgcaaccgagggtacaccctgtatggcttcacccactgtggagacaccaatgagtgcagcatcaacaacggaggctgtcagcaggtctgtgtgaacacagtgggcagctatgaatgccagtgccaccctgggtacaagctccactggaataaaaaagactgtgtggaagtgaaggggctcctgcccacaagtgtgtcaccccgtgtgtccctgcactgcggtaagagtggtggaggagacgggtgcttcctcagatgtcactctggcattcacctctcttcaggactgcaaggggcctactctgtcacctgtggctcttcctctcctctcaggaacaaacaacaaaaatcaaatgactctgcttttggggatgtcaccaccatcaggacaagtgtaacctttaagctaaatgaaggcaagtgtagtttgaaaaatgctgagctgtttcccgagggtctgcgaccagcactaccagagaagcacagctcagtaaaagagagcttccgctacgtaaaccttacatgcagctctggcaagcaagtcccaggagcccctggccgaccaagcacccctaaggaaatgtttatcactgttgagtttgagcttgaaactaaccaaaaggaggtgacagcttcttgtgacctgagctgcatcgtaaagcgaaccgagaagcggctccgtaaagccatccgcacgctcagaaaggccgtccacagggagcagtttcacctccagctctcaggcatgaacctcgacgtggctaaaaagcctcccagaacatctgaacgccaggcagagtcctgtggagtgggccagggtcatgcagaaaaccaatgtggtctgtgtcaacctggtgaatattctgcagatggctttgcaccttgccagctctgtgccctgggcacgttccagcctgaagctggtcgaacttcctgcttcccctgtggaggaggccttgccaccaaacatcagggagctacttcctttcaggactgtgaaaccagagttcaatgttcacctggacatttctacaacaccaccactcaccgatgtattcgttgcccagtgggaacataccagcctgaatttggaaaaaataattgtgtttcttgcccaggaaatactacgactgactttgatggctccacaaacataacccagtgtaaaaacagaagatgtggaggggagctgggagatttcactgggtacattgaatccccaaactacccaggcaattacccagccaacaccgagtgtacgtggaccatcaacccaccccccaagcgccgcatcctgatcgtggtccctgagatcttcctgcccatagaggacgactgtggggactatctggtgatgcggaaaacctcttcatccaattctgtgacaacatatgaaacctgccagacctacgaacgccccatcgccttcacctccaggtcaaagaagctgtggattcagttcaagtccaatgaagggaacagcgctagagggttccaggtcccatacgtgacatatgatgaggactaccaggaactcattgaagacatagttcgagatggcaggctctatgcatctgagaaccatcaggaaatacttaaggataagaaacttatcaaggctctgtttgatgtcctggcccatccccagaactatttcaagtacacagcccaggagtcccgagagatgtttccaagatcgttcatccgattgctacgttccaaagtgtccaggtttttgagaccttacaaatgactcagcccacgtgccactcaatacaaatgttctgctatagggttggtgggacagagctgtcttccttctgcatgtcagcacagtcgggtattgctgcctcccgtatcagtgactcattagagttcaatttttatagataatacagatattttggtaaattgaacttggtttttctttcccagcatcgtggatgtagactgagaatggctttgagtggcatcagcttctcactgctgtgggcggatgtcttggatagatcacgggctggctgagctggactttggtcagcctaggtgagactcacctgtccttctggggtcttactcctcctcaaggagtctgtagtggaaaggaggccacagaataagctgcttattctgaaacttcagcttcctctagcccggccctctctaagggagccctctgcactcgtgtgcaggctctgaccaggcagaacaggcaagaggggagggaaggagacccctgcaggctccctccacccaccttgagacctgggaggactcagtttctccacagccttctccagcctgtgtgatacaagtttgatcccaggaacttgagttctaagcagtgctcgtgaaaaaaaaaagcagaaagaattagaaataaataaaaactaagcacttctggagacataataatgtacatttattgccagccttcctcgttgcaagcttccaccctgcagcaaatgcactatgctgactctcgcaccttcagctgtgccctctgacactctgctggccattgcctgagggactggggagttcaggtgaaatgtaatttcctcagtggatgagaatcatcaattgtcatgaaaaggcgatttcaccaaccctgagtcggtattgccaagggttctactccccaagtgggcttcccatcacaagggatctgaggtccaggtgggtgtggtgaggcccagcctgtcatgtatgccttccttccccagtctcaaatgtgagagacggactctgctccgtggacatgagcagccccttgaagggtggtctcattctctaacagtaacagcactgccagaaacaccccctgaatgccacacgatcatctcactttgggtcaggccagcagggcctcacgatcatttcttgaacttgaattttcagtgttattgaatctttaaacatttaaatccccaagcaaaaatctcattacagtgaataaaaaggaatccaatcaaacaagccaccacttccctaatggatggactacctacaactatagtgttttgttatttcaataacaattctatgcacagccaattctccataccaaagtactaaaaaaatgtttaacaagcatttttgaagaaaacaaagagaacttcattatggttaaacttgcttttcatctcagtgttgctttttttaacctgttaataacattgattcttaaaaagtcctaatcaaacttctttatttggatgggccctaaatattcattttcagatttctcttcaaaagtttgaacatcatcattaaaaaaaaaatagcattaaaacaaaaaaaaaaaaaaaa

SEQ ID NO: 345 - DNA, 4664 bp, Homo sapiens
  source 1..4664: mol_type = other DNA
gcttgctcccaatcacaggagaaggaggaggtggaggaggagggctgcttgaggaagtataagaatgaagttgtgaagctgagattcccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaaaaaaaaaaaaaaaaaa

SEQ ID NO: 346 - DNA, 6789 bp, Homo sapiens
  source 1..6789: mol_type = other DNA
atattttagtaagggccaccctttaactcggcagcttgatgactataatgggcccagttgtctgcgggctgcggggagctaagtccccagattggaggaggctggctctggtcttcgatgcacaggagtggccgttatggaacgcagcagcagcgtgcagggtcaaagacagccggccccccatgtcagtggtctaggatggccagtgaaggcaccaacatcccaagtcctgtggtgcgccagattgacaagcagtttctgatttgcagtatatgcctggaacggtacaagaatcccaaggttctcccctgtctgcacactttctgcgagaggtgcctgcagaactacattcctgcccacagtttaaccctctcctgcccagtgtgccgccagacctccatcctgcccgagaaaggggtggccgcgctccagaacaatttcttcatcacaaacctgatggacgtgctgcagcgaactccaggcagcaacgctgaggagtcttccatcctggagacagtcactgctgtggctgcgggaaagcctctctcttgcccaaaccacgatgggaatgtgatggaattttactgccagtcctgtgagactgccatgtgtcgggagtgcacggagggggagcacgcagagcaccccacagttccactcaaggatgtggtggaacagcacaaggcctcgctccaggtccagctggatgctgtcaacaaaaggctcccagaaatagattctgctcttcagttcatctctgaaatcattcatcagttaaccaaccaaaaggccagcatcgtggatgacattcattccacctttgatgagctccagaagactttaaatgtgcgcaagagtgtgctgcttatggaattggaggtcaactatggcctcaaacacaaagtcctccagtcgcagctggatactctgctccaggggcaggagagcattaagagctgcagcaacttcacagcgcaggccctcaaccatggcacggagaccgaggtcctactggtgaagaagcagatgagcgagaagctgaacgagctggccgaccaggacttccccttgcacccgcgggagaacgaccagctggatttcatcgtggaaaccgaggggctgaagaagtccatccacaacctcgggacgatcttaaccaccaacgccgttgcctcagagacagtggccacgggcgaggggctgcggcagaccatcatcgggcagcccatgtccgtcaccatcaccaccaaggacaaagacggtgagctgtgcaaaaccggcaacgcctacctcaccgccgaactgagcacccccgacgggagcgtggcagacggggagatcctggacaacaagaacggcacctatgagtttttgtacactgtccagaaggaaggggactttaccctgtctctgagactctatgaccagcacatccgaggcagcccgtttaagctgaaagtgatccgatccgctgatgtgtctcccaccacagaaggcgtgaagaggcgcgttaagtccccggggagcggccacgtcaagcagaaagctgtgaaaagacccgcaagcatgtacagcactggaaaacgaaaagagaatcccatcgaagacgatttgatctttcgagtgggtaccaaaggaagaaataaaggagagtttacaaatcttcagggggtagctgcatctacaaatggaaagatattaattgcagacagtaacaaccaatgtgtgcagatattttccaatgatggccagttcaaaagtcgttttggcatacggggacgctctccggggcagctgcagcggcccacaggagtggctgtacatcccagtggggacataatcattgccgattatgataataaatgggtcagcattttctcctccgatgggaaatttaagacaaaaattggatcaggaaagctgatgggacccaaaggagtttctgtggaccgcaatgggcacattattgttgtggacaacaaggcgtgctgcgtgtttatcttccagccaaacgggaaaatagtcaccaggtttggtagccgaggaaatggggacaggcagtttgcaggtccccattttgcagctgtaaatagcaataatgagattattattacagatttccataatcattctgtcaaggtgtttaatcaggaaggagaattcatgttgaagtttggctcaaatggagaaggaaatgggcagtttaatgctccaacaggtgtagcagtggattcaaatggaaacatcattgtggccgactggggaaacagcaggatccaggtttttgatgggagtggatcatttttgtcctacattaacacatctgctgacccactctatggcccccaaggcctggccctaacttcagatggtcatgttgtggttgcagactctggaaatcactgtttcaaagtctatcgatacttacagtaatggtgggcaggtggatacccgcttccatggtcttgcactataaactggaatggatttctcaatgcgggaccagattatgactagagtttttatgccagaaggaatcattggtgaactttccaaggttatttctgaatgtaacaatttccttaaaaatgacttatccaatttctgtatttcacctttagggttaaaaaaaactcttctactgaatctataaaaactgcagttttacatctgtgaactatggcttaagggacaggatttatgtagctaaactaattttgcaaatcaaacagacacttaaaaaactagcatatgtaaaggtattcgttaatcctgtgaatggtagcttttgcacagaacttccaaaagcaaaacaaaaacaaaatctattgtagttatatacttcatttaacctaggtcacaagacccagggaatcttctaacctcacttttacagtaggtattactcttgtgacatttttttggttatcaacaactaaatataaattactttggaaaaagtaaggctgtcttgcaaaatgatcccagctctgattagcagccctctggagttcagaacttaagtatcagtgcaaatttctcaacctttctgggttagacaaagatccttttttgtgtgttcttttcaccacccctttggctcaccttgtatcagcaaacaaagtacttcttcagggaaacctgaaatttctaatgccttgaaaagcatattacaaaagtaatgctaccttttgggaaacaaactgccccgttaactccagatcattgcactggaatgtaatcaagaaagttagtcatgttttatgtaccatgttttcacacgtgtctcttctcttcgacttcctgaaagcgaaagctttacctcctgcaaatgtcagcacatgtagtaggacaccagtatcctaggacagagagccataagtagccctttggaggactgatggtgtcaaccaaaggcatgtgattgattaatgattcccccttagaaagcaagtgttaccaaagttgtgttatcttgaaagcattacaggtaagggcatgttatggttatttatcattgtttaatgaatagtagaggtgtcaagggactatgtatacatgattagggtaagatagaatgtattatatatatatatatatacacacacacatatatatagctgaatctttggtgtattgaaataggcagcactctgaaagacagaagcttcgtccagccactcttcagcacattcctttactaagcagtttaaagccgtcctagtggagcaagccctaaagcagatttaatttttgccattttccaagaatgacggtggtggcttttagtcagaaaatggccttctgtgctttcaaaaaaaaaaacaaaaaaaaaaccacacacacacataaaaaacccaacaggtcaaaataaaagttgaacttgagttacatttaatttaaatataaatgcattttgagaaatgttaagaacaatttagtcaatcgttcatctgtcattggtactgtaaaataagctgtggtctatttccactgtttaattttctactcagttctaccaaataggatgtcatgtttgacatttttgatagtgactttggggtcttcttcactgaaagcaccttagaactgtactataagaaaacatttcccctatgtataattatatgaatgtgatgtttattgcttattaatttataattcagtcattctctatataggacttcttaaaatttagaagggaaatctagctacttcaaattgtctgttaaatttattatgcccaaatcaacctctgaaaaaaggtttttccaggaagatttacatttaggtttaatatttttttagttaggtagagttttaaaaaatacttgagcctgtccgtgataaagctataaaattcaataactttttagaatgttaaatgaagacactgtttcctaacatcagtgagatacatctttgaatttaaacattcatatttactgagtacctactaggtaccaagtactcttttaggcactggaaatacagtgatggacaaaacaggtaaaaaatcgctgccccctcagagctgacattctggggtgggaatttcattttgccacgtactaacgttctgcacaaaagacaggctagactcttgtctagattgtttaaaagaaacttttcaaattggttacattaattttagtttattttcacaagtaaaaatggctttttatttagattctttctgtcccaggctgttgatcttaaaactagttgatttaaagagtttttttgcacaacatttcaattatatttgtgaacttagaaattaacttacaatctaaccagccatcatatcatatcctatcaggctagatatctcaatagtagactgaatacaaagctaattttttttacatgtcaatattggcacaaactggaatgaaagaatagtttgattcagacctgctccactatgtgttgctaaaacacatgctatgagcactccaggaaacactatattttttccaaaaaatatgtgattatatatgttaaagtatagataacatttcacacttggatacatatgtgcatttactgtatttcttggtaagcatatttttgggggaaagtgctgctgatatgatacaagtagacaaaatttaaatgaaattttgtcacattctatggaaaatggtttctggtaaactgagaaggatattaaaataagtggcttttttctgggctaccattattgtttgatttctctttgtcaagtgtatagaacctgtcatacattcatgataagtagcactgaaaaattactcattcaaatttcccctgggcacgtaaggcaaaatattgccggttgggatttcaaggtcagtgacgacgcatttcctcccagtacagaccccccagccccccttgctggacatggggaggcagagagtcacttgaccatccagaaatacatgactacaagtcctttatgactgtttgccattttttttaatggtacttagtattttgatcaaactttagtctccagaactaaacaagtccctaagtttccttattttaatttactgtgactagatttgaagcaaataaatactccagatccatgcagctagaacacacttgcttccactactaaatatacagggtatgtcctaacatggagttaactggaatagcagtacactagcaagtatctgtgaatccttagcactgacgggttaacagaaatgctttggtaatacctacttagttaattggaggaagtagtaaataaacattaggtaatctgcagattacttcaaatgggaaaaatctttttgtagactctatagtaccctctctattcactagcttctgaaaagggaggagtatttttagtttgacaatttaataatttaaaaacaagacatctccaggtaggaaaaaatgaaagctatttcatgcaaacattatctaatttagcttaaaagtgaaagtggtaatactgttggtttctgtaaatgttgcagggttttaaactttataattactttaatatttttgataactagaaatctagtattgccataaaggaaactaagtgcccatcaaagatttgtttggtataaataaagaattatttgttttgttttcaatgacagtaagctacaaatcatgatgcttaaaaactttctaaagatgaattgtgtggcagtgattggtctgtttgtggagaatgtatgaaagctattaatattctagaatagattaataaattggctatgttgttccaatgaatgtacagcacttccattaacttttgaaagcaacacagccttaaactcaatgcttttgctttatgacatgggaatgttctgtcatcaatggagtgtattcttgtaatagaattctttatatcgttctcaattctatagactttcaagcctatgtatgaatatgaaggggttttttttttttgctttgttttctttttagattttgtacattccatctttataggtctgtttcatatgttttatgtatagaacactaagtcttgcactctctgacattgatactgatatattctcgtcatttgttcttttatgaatcaaaatgttgactgcctatttaaagaaaagaatgaacgctgtgcatcaaagtgtttgtatgttcgtagctacatacgtaccacagtattttggatgctttagtctacaatgaaactttcaattaattctgtcttgaaacataggagaaacaggattcatgtgtatctctttaccatgcacaaaatctcaaatcattataataaagcttgttttctccatttgc

SEQ ID NO: 347 - DNA, 6330 bp, Homo sapiens
  source 1..6330: mol_type = other DNA
aggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatactgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaatagagtcatacagtagctcaaaaggcaaccataattctctttggtgcaggtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtgcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaactaaa

SEQ ID NO: 348 - DNA, 3637 bp, Homo sapiens
  source 1..3637: mol_type = other DNA
agtcctgggcgaagggggcggtggttccccgcggcgctgcgcgcggcggtaattagtgattgtcttccagcttcgcgaaggctaggggcgcggctgccgggtggctgcgcggcgctgcccccggaccgaggggcagccaacccaatgaaaccaccgcgtgttcgcgcctggtagagatttctcgaagacaccagtgggcccgttccgagccctctggaccgcccgtgtggaaccaaacctgcgcgcgtggccgggccgtgggacaacgaggccgcggagacgaaggcgcaatggcgaggaagttatctgtaatcttgatcctgacctttgccctctctgtcacaaatccccttcatgaactaaaagcagctgctttcccccagaccactgagaaaattagtccgaattgggaatctggcattaatgttgacttggcaatttccacacggcaatatcatctacaacagcttttctaccgctatggagaaaataattctttgtcagttgaagggttcagaaaattacttcaaaatataggcatagataagattaaaagaatccatatacaccatgaccacgaccatcactcagaccacgagcatcactcagaccatgagcgtcactcagaccatgagcatcactcagaccacgagcatcactctgaccatgatcatcactctcaccataatcatgctgcttctggtaaaaataagcgaaaagctctttgcccagaccatgactcagatagttcaggtaaagatcctagaaacagccaggggaaaggagctcaccgaccagaacatgccagtggtagaaggaatgtcaaggacagtgttagtgctagtgaagtgacctcaactgtgtacaacactgtctctgaaggaactcactttctagagacaatagagactccaagacctggaaaactcttccccaaagatgtaagcagctccactccacccagtgtcacatcaaagagccgggtgagccggctggctggtaggaaaacaaatgaatctgtgagtgagccccgaaaaggctttatgtattccagaaacacaaatgaaaatcctcaggagtgtttcaatgcatcaaagctactgacatctcatggcatgggcatccaggttccgctgaatgcaacagagttcaactatctctgtccagccatcatcaaccaaattgatgctagatcttgtctgattcatacaagtgaaaagaaggctgaaatccctccaaagacctattcattacaaatagcctgggttggtggttttatagccatttccatcatcagtttcctgtctctgctgggggttatcttagtgcctctcatgaatcgggtgtttttcaaatttctcctgagtttccttgtggcactggccgttgggactttgagtggtgatgcttttttacaccttcttccacattctcatgcaagtcaccaccatagtcatagccatgaagaaccagcaatggaaatgaaaagaggaccacttttcagtcatctgtcttctcaaaacatagaagaaagtgcctattttgattccacgtggaagggtctaacagctctaggaggcctgtatttcatgtttcttgttgaacatgtcctcacattgatcaaacaatttaaagataagaagaaaaagaatcagaagaaacctgaaaatgatgatgatgtggagattaagaagcagttgtccaagtatgaatctcaactttcaacaaatgaggagaaagtagatacagatgatcgaactgaaggctatttacgagcagactcacaagagccctcccactttgattctcagcagcctgcagtcttggaagaagaagaggtcatgatagctcatgctcatccacaggaagtctacaatgaatatgtacccagagggtgcaagaataaatgccattcacatttccacgatacactcggccagtcagacgatctcattcaccaccatcatgactaccatcatattctccatcatcaccaccaccaaaaccaccatcctcacagtcacagccagcgctactctcgggaggagctgaaagatgccggcgtcgccactctggcctggatggtgataatgggtgatggcctgcacaatttcagcgatggcctagcaattggtgctgcttttactgaaggcttatcaagtggtttaagtacttctgttgctgtgttctgtcatgagttgcctcatgaattaggtgactttgctgttctactaaaggctggcatgaccgttaagcaggctgtcctttataatgcattgtcagccatgctggcgtatcttggaatggcaacaggaattttcattggtcattatgctgaaaatgtttctatgtggatatttgcacttactgctggcttattcatgtatgttgctctggttgatatggtacctgaaatgctgcacaatgatgctagtgaccatggatgtagccgctgggggtatttctttttacagaatgctgggatgcttttgggttttggaattatgttacttatttccatatttgaacataaaatcgtgtttcgtataaatttctagttaaggtttaaatgctagagtagcttaaaaagttgtcatagtttcagtaggtcatagggagatgagtttgtatgctgtactatgcagcgtttaaagttagtgggttttgtgatttttgtattgaatattgctgtctgttacaaagtcagttaaaggtacgttttaatatttaagttattctatcttggagataaaatctgtatgtgcaattcaccggtattaccagtttattatgtaaacaagagatttggcatgacatgttctgtatgtttcagggaaaaatgtctttaatgctttttcaagaactaacacagttattcctatactggattttaggtctctgaagaactgctggtgtttaggaataagaatgtgcatgaagcctaaaataccaagaaagcttatactgaatttaagcaaagaaataaaggagaaaagagaagaatctgagaattggggaggcatagattcttataaaaatcacaaaatttgttgtaaattagaggggagaaatttagaattaagtataaaaaggcagaattagtatagagtacattcattaaacatttttgtcaggattatttcccgtaaaaacgtagtgagcacttttcatatactaatttagttgtacatttaactttgtataatacagaaatctaaatatatttaatgaattcaagcaatatatcacttgaccaagaaattggaatttcaaaatgttcgtgcgggtatataccagatgagtacagtgagtagttttatgtatcaccagactgggttattgccaagttatatatcaccaaaagctgtatgactggatgttctggttacctggtttacaaaattatcagagtagtaaaactttgatatatatgaggatattaaaactacactaagtatcatttgattcgattcagaaagtactttgatatctctcagtgcttcagtgctatcattgtgagcaattgtcttttatatacggtactgtagccatactaggcctgtctgtggcattctctagatgtttcttttttacacaataaattccttatatcagcttgaaaaaaaaaaaaaaaaaa

SEQ ID NO: 349 - DNA, 1686 bp, Homo sapiens
  source 1..1686: mol_type = other DNA
gcttaagttgacctctgggtccggaatcgcgggcaaagatggcggcggccaggtgttggaggcctttgctacgcggtccgaggctttcattgcacaccgcggctaatgccgccgccacggctacagaaacgacctgccaagacgtcgcggcgacccccgtcgcgcggtacccgccgattgtggcctccatgacagccgacagcaaagctgcacggctgcggcggatcgagcgctggcaggcgacggtgcacgctgcggagtcggtagacgagaagctgcgaatcctcaccaagatgcagtttatgaagtacatggtttacccgcagaccttcgcgctgaatgccgaccgctggtaccagtacttcaccaagaccgtgttcctgtcgggtctgccgccgcccccagcggagcccgagcccgagcccgaacccgaacctgaacctgcgctggacctcgcggcgctgcgtgcggtcgcctgcgactgcctgctgcaggagcacttctacctgcggcgcaggcggcgcgtgcaccgttacgaggagagcgaggtcatatctttgcccttcctggatcagctggtgtcaaccctcgtgggcctcctcagcccacacaacccggccctggccgctgccgccctcgattatagatgcccagttcatttttactgggtgcgtggtgaagaaattattcctcgtggtcatcgaagaggtcgaattgatgacttgcgataccagatagatgataaaccaaacaaccagattcgaatatccaagcaactcgcagagtttgtgccattggattattctgttcctatagaaatccccactataaaatgtaaaccagacaaacttccattattcaaacggcagtatgaaaaccacatatttgttggctcaaaaactgcagatccttgctgttacggtcacacccagtttcatctgttacctgacaaattaagaagggaaaggcttttgagacaaaactgtgctgatcagatagaagttgtttttagagctaatgctattgcaagcctttttgcttggactggagcacaagctatgtatcaaggattctggagtgaagcagatgttactcgaccttttgtctcccaggctgtgatcacagatggaaaatacttttcctttttctgctaccagctaaatactttggcactgactacacaagctgatcaaaataaccctcgtaaaaatatatgttggggtacacaaagtaagcctctttatgaaacaattgaggataatgatgtgaaaggttttaatgatgatgttctacttcagatagttcactttctactgaatagaccaaaagaagaaaaatcacagctgttggaaaactgaaaaagcatatttgattgagaactgtgggaatatttaaattttactgaaggaacaataatgatgagatttgtaactgtcaactattaaatacattgatttttgagacaaatatttcttatgtcaacctgttattagatctcttactctgctcaaattcatcactgaaagatttaattttagttaccttttgttgatttaaaaataattgcatttgtatattgctaactgataagacaaattgagttattgagctattaaatgcacattttaatataaatgcagaaatcccaaataaaatgctaacatactgaaaaaaaaaaaaaaaaaa

SEQ ID NO: 350 - DNA, 5361 bp, Homo sapiens
  source 1..5361: mol_type = other DNA
tttctccttccctccacgggccgggtgagaaagtagccgggggctatcccgacccggcggttcttggggagggggccgaacaagaaaagggaggagatggagataacttccccggatttagcttttttgtctttgtttttgttctcaccacttccatcggatgactggagagtaaaagggaacccggagcggggtggcgagcagcgctttgagaaaatgcaggagtgtgtttggagacgcgtaaagttgcctttcaagctctggcctccgggcacgcgatgctccgcggcgggctgactcagggctgccttgggcctccctgccaccctcctggaaatgatgcaagtcctgactgtcacctggatccctgcagcccagcctggaatgcgtctggattaggggaaagacgagaaacgacactccaggtgttgcacggcccaccaaagcgggaagatagggcagttgctcagaccaaatactgtatctagtgcttctgctcctatcttcaatcgtggggttctttttaatgcaaagtgtcacaaggccaggaattcccatgtgtgctcagttggcccacagcatcattgtgcctaggaaactgcttcaatttatcaagtcctctgggctgggaatctcactgaattccaaacggcggaaagaggaaactttcccaacccgatgtgggtgtgacgcgagccaggggccccagggacactgtcccagagcacaccgtccccctttaacagcaactggagcttggattcgctcttatattgtacagtcctttcgaccattgccctggagcacccgcacacgcgcacgcatctccggccgcgctcacacacactcatacacacgcacgcaaacgcgtggccgccgccaggtcggcaactttgtccggcgctcccagcggcgctcggcttcctcctgtagtagttgagcgcaggccccgcctcccggccgtgttgtcaaaagggccggggtctcggattggtccagccgccgggacaacacctgctcgactccttcattcaagtgacaccagagcttccagggatatttgaggcaccatccctgccattgccgggcactcgcggcgctgctaacggcctggtcacatgctctccggagagctacgggagggcgctgggtaacctctatccgagccgcggccgcgaggaggagggaaaaggcgagcaaaaaggaagagtgggaggaggaggggaagcggcgaaggaggaagaggaggaggaggaagaggggagcacaaaggatccaggtctcccgacgggaggttaataccaagaaccatgtgtgccgagcggctgggccagttcatgaccctggctttggtgttggccacctttgacccggcgcgggggaccgacgccaccaacccacccgagggtccccaagacaggagctcccagcagaaaggccgcctgtccctgcagaatacagcggagatccagcactgtttggtcaacgctggcgatgtggggtgtggcgtgtttgaatgtttcgagaacaactcttgtgagattcggggcttacatgggatttgcatgacttttctgcacaacgctggaaaatttgatgcccagggcaagtcattcatcaaagacgccttgaaatgtaaggcccacgctctgcggcacaggttcggctgcataagccggaagtgcccggccatcagggaaatggtgtcccagttgcagcgggaatgctacctcaagcacgacctgtgcgcggctgcccaggagaacacccgggtgatagtggagatgatccatttcaaggacttgctgctgcacgaaccctacgtggacctcgtgaacttgctgctgacctgtggggaggaggtgaaggaggccatcacccacagcgtgcaggttcagtgtgagcagaactggggaagcctgtgctccatcttgagcttctgcacctcggccatccagaagcctcccacggcgccccccgagcgccagccccaggtggacagaaccaagctctccagggcccaccacggggaagcaggacatcacctcccagagcccagcagtagggagactggccgaggtgccaagggtgagcgaggtagcaagagccacccaaacgcccatgcccgaggcagagtcgggggccttggggctcagggaccttccggaagcagcgagtgggaagacgaacagtctgagtattctgatatccggaggtgaaatgaaaggcctggccacgaaatctttcctccacgccgtccattttcttatctatggacattccaaaacatttaccattagagaggggggatgtcacacgcaggattctgtggggactgtggacttcatcgaggtgtgtgttcgcggaacggacaggtgagatggagacccctggggccgtggggtctcaggggtgcctggtgaattctgcacttacacgtactcaagggagcgcgcccgcgttatcctcgtacctttgtcttctttccatctgtggagtcagtgggtgtcggccgctctgttgtgggggaggtgaaccagggaggggcagggcaaggcagggcccccagagctgggccacacagtgggtgctgggcctcgccccgaagcttctggtgcagcagcctctggtgctgtctccgcggaagtcagggcggctggattccaggacaggagtgaatgtaaaaataaatatcgcttagaatgcaggagaagggtggagaggaggcaggggccgagggggtgcttggtgccaaactgaaattcagtttcttgtgtggggccttgcggttcagagctcttggcgagggtggagggaggagtgtcatttctatgtgtaatttctgagccattgtactgtctgggctgggggggacactgtccaagggagtggcccctatgagtttatattttaaccactgcttcaaatctcgatttcactttttttatttatccagttatatctacatatctgtcatctaaataaatggctttcaaacaaagcaactgggtcattaaaaccagctcaaagggggtttaaaaaaaaaaaaccagcccatcctttgaggctgatttttcttttttttaagttctattttaaaagctatcaaacagcgacatagccatacatctgactgcctgacatggactcctgcccacttgggggaaaccttatacccagaggaaaatacacacctggggagtacatttgacaaatttcccttaggatttcgttatctcaccttgaccctcagccaagattggtaaagctgcgtcctggcgattccaggagacccagctggaaacctggcttctccatgtgaggggatgggaaaggaaagaagagaatgaagactacttagtaattcccatcaggaaatgctgaccttttacataaaatcaaggagactgctgaaaatctctaagggacaggattttccagatcctaattggaaatttagcaataaggagaggagtccaaggggacaaataaaggcagagagaagagacagaactaaaaatacgaggaaaggagagtgaggattttcattaaaagtctcagcagtgggtttcttgggttatttaaaacatcacctaaataggccttttcttcctaattggccatcaaattaaagcctatcctttctcaagcaggagctggtattgtagggagtggccgggtattctgggctgggctcttctggagtagggggtcagcaaacattgtctgcaaagggccagatactgaatccagtactttcagtttggcgagccgtgaggtctctgtcgaaactactcaactctgccgtcctagcacaaaagcagccatagacaacacacaaacgagagggcttggctcccttccaggaagatttatttaacaggctcccagctgaatttcactcacaggacacagtttactgatctctgttctagtgagtgggtcaaaaagcatatgcatccttatccgtcaactcatcagctcttcctcaaggcaacctgaggccagacaccaagaaaccaagcgtatctgctctaaaatgacttgttcctggggaatgccttcaaccaaaacacagctagtatttctatgccccaaatccaatcccagtctttcatgatccatgccggcggttgggtggggaggggaatcattggttgggggaagggaggaaaccccacctccagcccccgccaccgggctccctgggcacccagcaagatctggggctgcagagaacagaagagctggtgcacttaatccagctctgcccttggggggaggaggacctgtgtgtcaggctctgccatgggaacgagtgtaaaccgtggctgtctcctgcagtgagccaccgcggcaggcacgttgactgttttactgacatcactcaaaagctaaagcaataacattctcctgcgttgctgagtcagctgttcatttgtccgccagctcctggactggatgtgtgaaaggcatcacatttccattttcctccgtgtaaatgttttatgtgttcgcctactgatcccattcgttgcttctattgtaaatatttgtcatttgtatttattatctctgtgttttccccctaaggcataaaatggtttactgtgttcatttgaacccatttactgatctctgttgtatatttttcatgccactgctttgttttctcctcagaagtcgggtagatagcatttctatcccatccctcacgttattggaagcatgcaacagtatttattgctcagggtcttctgcttaaaactgaggaaggtccacattcctgcaagcattgattgagacatttgcacaatctaaaatgtaagcaaagtagtcattaaaaatacaccctctacttgggctttatactgcatacaaatttactcatgagccttcctttgaggaaggatgtggatctccaaataaagatttagtgtttattttgagctctgcatcttaacaagatgatctgaacacctctcctttgtatcaataaatagccctgttattctgaagtgagaggaccaagtatagtaaaatgctgacatctaaaactaaataaatagaaaacaccaggccagaactatagtcatactcacacaaagggagaaatttaaactcgaaccaagcaaaaggcttcacggaaatagcatggaaaaacaatgcttccagtggccacttcctaaggaggaacaaccccgtctgatctcagaattggcaccacgtgagcttgctaagtgataatatctgtttctactacggatttaggcaacaggacctgtacattgtcacattgcattatttttcttcaagcgttaataaaagttttaaataaatggcaaaaaaaaaaaaaaaaaa

SEQ ID NO: 351 - DNA, 2301 bp, Homo sapiens
  source 1..2301: mol_type = other DNA
tccagtgagttgcaagcctaatgctcacctgcagagacagaattcctgagtgaacgaacagagcagctcctcttccatctccagggtctcattctgttgcccaggctggagtgcagtggtgcgatctcggctcacagcaacctctctgcctccagaaatgacctccaccttcaacccccgagaatgtaaactgtccaagcaagaagggcaaaactatggcttcttcctgcgaattgagaaggacaccgagggccacctggtccgggtggttgagaagtgtagcccagcagagaaggctggccttcaagatggagacagagttcttaggatcaatggtgtctttgtggacaaagaagaacatatgcaggttgtggatctggtcagaaagagtgggaattcagtgactttactagttctggatggggattcctatgagaaagcagtgaaaacacgggtggacttgaaagagttgggtcaaagtcagaaggagcaaggtttgagtgataatatactttcccctgtgatgaatggaggtgtgcaaacttggacccagccccggctctgctatctcgtgaaggaaggaggcagctatggcttctctctgaaaactgtccaaggtaaaaagggggtgtacatgactgatattacacctcaaggtgtggctatgagagctggagttctggctgatgatcacttgattgaagtgaatggagagaatgtagaggatgccagccatgaggaagtggttgaaaaggtgaagaagtcaggaagccgtgtcatgttcctgctggtggacaaagaaactgacaagcgtcatgttgagcagaagatacaattcaaaagagaaacagccagtttgaaactgttaccccaccagccccgaattgtggagatgaagaaaggaagcaatggctatggtttctatctgagggcaggctcagaacagaaaggtcaaatcatcaaggacatagattctggaagtccagcagaggaggctggcttgaagaacaatgatctggtagttgctgtcaacggcgagtctgtggaaaccctggatcatgacagtgtggtagaaatgattagaaagggtggagatcagacttcactgttggtggtagacaaagagacggacaacatgtacagactggctcatttttctccatttctctactatcaaagtcaagaactgcccaatggctctgtcaaggaggctccagctcctactcccacttctctggaagtctcaagtccaccagatactacagaggaagtagatcataagcctaaactctgcaggctggctaaaggtgaaaatggctatggctttcacttaaatgcgattcggggtctgccaggctcattcatcaaagaggtacagaagggcggtcctgctgacttggctgggctagaggatgaggatgtcatcattgaagtgaatggggtgaatgtgctagatgaaccctatgagaaggtggtggatagaatccagagcagtgggaagaatgtcacacttctagtctgtggaaagaaggcctatgattatttccaagctaagaaaatccctattgtttcctccctggctgatccacttgacacccctccagattctaaagaaggaatagtggtggagtcaaaccatgactcgcacatggcaaaagaacgggcccacagtacagcctcacattcttcttccaattctgaagatacagagatgtgatgaaaacaagtaatagctttggctgtttatttgatagctgtttctgggtatttaataggaatcctttctcaaggaatgagttgtgacctgtttactgtctctttagaagaaaaactccactggaaaccattcaccatgtgtgattgtcttctgttatcatttgtcttacaggcggctattgcagacggctaatttatgcttaacttaggaagagataaggcaagagctagatttttttcatgtgatcttttccaagcttcaacttaacttaactacatttctctgtatgatgatgtctcttacttctacaggttccttgagcaccaaagatgattcataactctgtataggtgacagctgcttataaaagcatcttagcagataagcctattaaaattgtgcttttgtaacaatgttgtggttgctagaataaataccattaacaaatgccttttgagtatgcttgatagtgcttttgttttggattcactttttatgctttaaccttcatttgcctctagaaacccaaaacacaataaagtacagaataagaccttagtaataaaattcagaattttcttaaa

SEQ ID NO: 352 - DNA, 1821 bp, Homo sapiens
  source 1..1821: mol_type = other DNA
gagagcacttcctcatagaccttggatgtgggaggattgcattcagtctagttcctggttgccggctgaaataacctgaattcaagccaggaagaagcagcaatctgtcttctggattaaaactgaagatcaacctactttcaacttactaagaaaggggatcatggacattgaagcatatcttgaaagaattggctataagaagtctaggaacaaattggacttggaaacattaactgacattcttcaacaccagatccgagctgttccctttgagaaccttaacatccattgtggggatgccatggacttaggcttagaggccatttttgatcaagttgtgagaagaaatcggggtggatggtgtctccaggtcaatcatcttctgtactgggctctgaccactattggttttgagaccacgatgttgggagggtatgtttacagcactccagccaaaaaatacagcactggcatgattcaccttctcctgcaggtgaccattgatggcaggaactacattgtcgatgctgggtttggacgctcataccagatgtggcagcctctggagttaatttctgggaaggatcagcctcaggtgccttgtgtcttccgtttgacggaagagaatggattctggtatctagaccaaatcagaagggaacagtacattccaaatgaagaatttcttcattctgatctcctagaagacagcaaataccgaaaaatctactcctttactcttaagcctcgaacaattgaagattttgagtctatgaatacatacctgcagacatctccatcatctgtgtttactagtaaatcattttgttccttgcagaccccagatggggttcactgtttggtgggcttcaccctcacccataggagattcaattataaggacaatacagatctaatagagttcaagactctgagtgaggaagaaatagaaaaagtgctgaaaaatatatttaatatttccttgcagagaaagcttgtgcccaaacatggtgatagattttttactatttagaataaggagtaaaacaatcttgtctatttgtcatccagctcaccagttatcaactgacgacctatcatgtatcttctgtacccttaccttattttgaagaaaatcctagacatcaaatcatttcacctataaaaatgtcatcatatataattaaacagctttttaaagaaacataaccacaaaccttttcaaataataataataataataataaaaaatgtattttaaagatggcctgtggttatcttggaaattggtgatttatgctagaaagcttttaatgttggtttattgttgaattcctagaaaagttttattggtagatgagtaaataaaatattgtaaaaaaacttattgtctataaagtatattaaaacattgttggctaatataatttgaaaaaaagtggttttttggaagacttaggatattatggtgctacataatttttcctcgatgctctcttcctctcatctttcttgtctcttaaattactttacttccttgcacactttgccatacaagaatgaacatgagcttttcttgtgtagatctgagttgaaatcctgtggacactgggcgaattactttttagatctgtagctctgactcctcaggcataaaatgggaataatgcttttacagtttagtggcggaactaaactcccaaaattatttgttatatggatcaagtaataacgtcagtaatgtttttggtacaaagtcattatttaataaaagttattgttccatcttgcttgcccccccaaaaaaaaaaaaaaaaa

SEQ ID NO: 353 - DNA, 12499 bp, Homo sapiens
  source 1..12499: mol_type = other DNA
ccgctgcatggcgctgagatggcgggggcgccgcgcggcggaggcggcggcggaggcggcgcgggcgagcccgggggcgccgagcgggcggccgggacaagccgccggcgcgggctccgggcgtgcgacgaggagttcgcttgcccagagctggaggcgctgttccgcggctacacgctgcggctggagcaggcggccacgctgaaggcgctggccgttctcagcctgctggcgggcgcgctggcgctggccgagctgctgggcgcgccggggcccgcgcccggcctggccaagggctcacacccggtgcactgcgtcctcttcctggcgctgctcgtggtaaccaacgtccggtccctgcaggtgccccagctgcagcaggtcggccagctggcgctgctcttcagcctcaccttcgcgctgctctgctgtcctttcgcgctgggcggccccgcccggggttccgccggggccgctggggggccagcgaccgccgaacaaggggtttggcagctccttttggtcaccttcgtgtcctatgccttgctgcccgtgcgcagcctgctggccataggctttgggctcgtggtggctgcgtcgcacttgctggtcacagccaccttggtccccgccaagcgcccacgtctctggaggacgctcggtgccaatgccttgctcttcgtcggtgtgaacatgtatggggtctttgtgcggattctgactgagcgttcacagaggaaggcgttcctgcaggcccggagctgcattgaggaccgactgaggctggaggatgagaacgagaagcaggagcggctcctcatgagcctcctgccccggaacgttgccatggagatgaaggaggacttcctgaagccccctgagaggattttccacaagatttacatccagaggcacgacaatgtgagcatcctgtttgctgacatcgtgggtttcacgggcttggcatcccagtgcacagcccaggagctggtgaaactcctcaatgagctcttcggcaagttcgatgaattagccacggagaaccactgtcgccgcatcaagattctcggggactgctactactgcgtgtcgggcctcacccagcccaagactgaccatgcccactgctgtgtggagatgggactcgacatgattgataccatcacatctgtggctgaagccaccgaggtggatctgaacatgcgtgtgggtctgcacacgggcagggtcctctgtggtgtcctgggcttgcgcaagtggcagtacgacgtgtggtccaatgatgtgaccttggccaatgtcatggaagccgctggcctgccagggaaggttcatatcacaaagacgaccctagcgtgcttgaatggggactacgaggtagaaccgggttacggacatgagaggaacagtttcttgaaaactcataacatcgaaaccttttttattgtgccatcccatcgccgaaagatatttccaggcctgattctctcagatataaaaccggccaaaaggatgaagttcaagactgtctgctacctgctggtgcagctcatgcactgccggaaaatgttcaaggccgagatccccttctccaatgtcatgacctgcgaggacgatgacaagcggagggcattaagaacagcctcggaaaaactcagaaaccgctcatctttttctaccaacgttgtctacaccaccccgggcactcgcgtcaacaggtacatcagccgcctcttagaagcccgccagacagagctggagatggcagacctgaacttctttaccctgaagtacaaacatgtcgaacgggagcaaaagtaccaccagcttcaggacgagtatttcaccagcgccgttgtcctcaccctcatcctggctgccttatttggccttgtctaccttctaatattcccacagagtgtggtcgtcctgctcctgctagtattctgcatctgcttcctggtggcctgtgtcctgtacctgcacatcacccgggtccagtgttttccagggtgcctgacgattcagattcgcactgtcctgtgtattttcatagtggtcttaatctactcagtagcccaaggttgtgtggtgggctgcctgccttgggcctggagctccaagcccaacagttccctggtggtcctttcgtctgggggccagcgcacagccctgcccaccctgccctgcgagtctacacaccatgccctgctctgctgcctggtgggcaccctcccgctagccatatttttccgggtgtcctccttgccaaaaatgatcctgctctccgggctcaccacgtcctacatcctcgttctggagctcagcggatacaccaggactgggggtggtgccgtctccgggcgcagctacgagccgattgtggccatcctgctcttctcctgtgcgctggccctgcatgccaggcaggtggacatcaggctgaggctggactacctctgggccgcacaggcagaggaggagcgagaggacatggagaaggtgaagctggacaacaggcgcatcctcttcaacctcctgccggcccacgtcgcccagcacttcctcatgtccaaccctcggaacatggacctctactaccagtcctactcccaggtgggcgtcatgtttgcctccatccccaacttcaatgacttctacatcgagctggacggcaacaacatgggggtggagtgtctgcggcttctcaacgagatcatcgccgactttgacgagctcatggaaaaagacttttacaaggacatagagaagatcaagaccatcgggagcacctacatggccgctgtggggctagcgcccacctcggggaccaaggctaagaagtccatctcctcccacctgagcacgctggcggactttgccattgagatgtttgacgttctggatgaaatcaactaccagtcttacaacgactttgtcctccgagttggcatcaatgttggccctgtggtggctggagtgattggcgctcgcaggccccagtacgacatctggggaaacacagtcaacgtggccagtcggatggatagcacaggggtccagggcagaatccaggtgactgaggaagtccaccggctgctgagaaggtgcccctaccactttgtgtgccgaggcaaagtcagtgtcaagggcaaaggcgagatgttgacatactttctagaaggcaggactgatggaaacggctcccaaatcaggtccctgggcttggatcggaaaatgtgtccatttgggagagctggccttcagggcagacgtccccccgtgtgccccatgcctggcgtctcagtcagggctgggctccctccacactccccaggccagtacctgccctctgcagcagctgggaaggaggcttagtggagcccacgtgggcctctggggtgcacatggggtgggaatgctccgggggtgacacaggccacggtggctccagccaggaccagccagaccagcagagcagggagccacttgccagggtggaggaggagcattgtccaggcatggcctgtggcccgagggccaaccaccgagcaggcacagcacagcagtgactcggtgaggggaggacacccgactgtgcacacttccaggcctccctggagaggtgtccactccatccttccctccgtggcgtagggagcactgtttctttccagcaagcctgttcaggtttggccaggtcggcatcaatgtaaggaccttcagagcatcccagggttacagcaagagccactgaggtgtggctggcagagcaactgaggagctctgcgagagtgtgccctctgagacagccctgtccgccccgcccaggtgggacctgacatacaggcggctccagccctgggcagcggcttcaagagccagagtgggaagtcaggcctctctgggaaggtcacaaggcctggggttgttcacaccacaggcacaatacagagctgtccactcttgaactggaccgtctggggagaattgagaagctgcctctcactgcatggatagtggtggctgctggctttcaagcgtggttttttgctaagagccagaggcaggtcttctggagttctcaggggggccctgtgaccccctctccacaaccatggccagcatcggcttcttgctgcacctgtgcattctccacccctccgccaatggcttccagctcctaaaacacctggacattccccatcctgcaccggcactgtgtttgttcagagaaaacgaagggtgtcatttcttcttttccctgtggcatcgtggatcctttacatttagcaacaacagtgtgaggctccacttctgttgaatatgagcggctgtcacacccatgagcaagtttccttgatggaagatgctgtttctccggcagaaccaaccctccaggacgaagtttcagaattggctgatggtcgtgagtgcaggtgtccctgttgtaatttgcagggaaattgactgcaggtgatagtgttgacgtctgacgtctgacaacctcctatgtgcatgtctaaatagtcacaatgaagatgaaaataaggatgatggggacggggtccaggagggccactgggaaggcactatccagaaatagtggacagaggtctggcatgtggaaaccttggatctcggtcacagggaggaaggcagctagaaatgggagggaagcttctgggtaggcaccatagtaggggaatgtctaaagacccactgctctgagatgtggtcagagaggtttctgtggactgcagatgaggcctgagccctgcctatgctcaaagccagcagggttgggggtccgcctcctcactgctcctcaacagctgtaaccctttcctgagggccccgacttttcagaataggagcagagagaagcccagacttccttccctggagtgtagacctgtgtgtgggacctgctccaggctgatccctgtccgcacaggtgggtgatgtccttggctgctgccgtcagaggcagccccctgcacagggaggggctgatcctgcctcagctgtgtggccatgtgggggcagcaggggcctgggccatagctgggggaggccagaccctgagggctttgcagggcggccagtgcagtgatgcctggcgcttccagctgagacaggagtccggacagaggtgggcagggtggatggcttcgggcagggaaggcgggtagaggctgcatgtgggggcccctgtctgcctgtccttagggtgtggattaggctctggcttctgtgatggtgcaagaggcaggatggtctccagacaccttcttgcagagtgattttgaccaatgcctttccagcaccttcccctggttatgacagggagctcccaggccaggccacatgaccccacaggtaggctgaccccatggacccactggagactttgagggcagagctcgggcacctttcatcttccttgggtggtcctcatgtcgctgtccgctcatgttcctggtgttctggcatcaccttgtgccggtgggggctgggctcccgtgaggtcctagggcagaaggaggtgcttgcagggcctctcgctgctactgggacatccacaggacctaatgtttatgtggaatctccaggctgccatcgagggctcctgagctcatcctgaggacccagggtccatctgagctggctgtgggctgagatcctggctggggccctgctgtcaacctctcctccaggccctgccttgtccctgctcctgtgctttcctctcttcaggacaaatggtttgtccttatggtcccctttccactaggattttctcatcctcatctgtctatcccagccggatttaaagcagtcacttcagacaggagccacagggagcctgtgagggagtagtgcggtggtggtgagtgtggctgccctggctcccccagctcaccgtggtgctgtccttgacccacgagctgttgagaagggtgcagagcccctggcacagccaaccctgttgcccccagtgccgctgcccagcttcggccatccaggtggtgggtagacagaggagtgaaccccaaaggtgtccatgccccagttctaagccctgtggccaggtgctgtcacacagcaaaggggactttgcaggtggaattagggttagggatctaaggatgggaggggaacttggtttatctgggtgggctcagtgaccccttggaatggctgagggaggcaggagagcctggaaagattcgaagtgtcacagagatggtgccgtcattgctggcttaaagacaaggggccaggagccaacaccagaggccactccacaaccccagcgccagtcagcaaggaagccgggtgtcgctcacacaaccagaaggagctgagtttggcaacaacccaagtgagcctgaagcagattcgccccagagcctccaggcgtgagtggagcccagtccagcgacaccttggtttggcctgtgagactgagaagagagccagccacgccatgctgtgcctggggtccacagagccgggaggtcacacatgtacatcctggaagctgcccaaggtgtggccacttgtagggcagtgagaggaagccgtcgcggggaccatgctgccttctctgggctcacgactgttctggaaaggaccccatggcagacttggcaaggtctccacttcaggggcaaacatctccatcatcagcaactccaaaccccagccacttcctgggagttcaggaactgtgtttagccataatgtcccttggggtttggtgaaaaacaacttcctttttatttgtgggaaaactgtattatattgccttatttatttttaatcatgtacaatattcatgtacaaatgttagagccattcgggtaggattctctctaaattatttattgaagaggctttgtaaatagtgcaccagtgacaggtgctgcctgtttactcgtaccaaaaatgaagacaagccccacacccacctcagcccttgcttggccatgctgtggccctttggaaatgaccctgtgtgctcccctcgcgacccttgcatgcctttcatgcctgatgccgtagagtagaacacagccccggaatgcttccagttgcttgcaagggaccatgccggcccgggtggtccatgcctgcggggtgtctgtatcctgcaggaggacgcccctacagacccagcaacccaagttgccagcccgtgtttctcagactttaaagccaccagggcttctggaacacctgtcatagaagcaataactctaactctatactgtagagatgagtctggtccgaagcagattagtaatttagagataagccatgcatacaagtttgccccattctctggcttggggtggtgggaggcggggagctgccactcccaaagcctccaccccttcctgaagacccagctatgaaaccgcagttcctgtgggttgggctgcccaggctggtcagagctcagcaccccaggtctggtgagcagacagagccggcgttaaaccccgatcaggtcattgtcaccacacccttgcttggatgaggaaggaaggaggcaaaggcacccactccatccaagatccgttccaattgatgagcctcagtactggcctcacactgaccaacggtggtatgctgcagtttgttggtttggtgcagagtctaaaggataacatatcaaggcttacctgtgataggccattttaaggaaatggtggcactctgaaggctgtccccattcaccttccacaatgctggtaccatcctggtagaagagccctctgaacaggacacctgttcaggcctgggggccccatgctaggcttgcctgggtgtgagaaggtcgaactgggtggcttaggccttcaaattgagcttgggggtgtccaagagaggggtcttattcgaggaactcacataccccaaataaccatcagtgcacaaatcattccatgctctttggcaccaggcaggagatgctgcaggcaggtgtccgtagttggcaaaaatgggcagtaggggaaatggagttgcttcaacctttctcagtcccggccctcactgcacccaccaagtcaagcagagctgggtgggctgagaccagcatcccagctggactgagcctggggggaggggctggagccaaggagcccccctgggtgggggcatcaggagcaggggcagggcctgaggactgctccccctgcatggagggcaggccaggatggagcccagggtagactttgaggaaggctcagtgggctcagcagaaggaaggctcttctcatagttgtggtttttcacagatggatttgggtgatctgagtgtggtcaggccctgccgttggggaatgaatgccacacggggtttgtctgtaagctgcagtgtctgtgattctgtgtgaactgacagcagggagctgaaggacaggccccggccttgggagaggacacgggcatgggctgctggatgcagggctcccagggctgtgaaggcagaggggtctctggaggcagacagggtgggctgggggccagcagcagggggcagagccccagccagggcagctgcttcctgcacatagacattcctcctgcgcagggccaagtgcccatgagcacagccccagacagacacggtggggagcttggagccctccgctcacctagaggagccctgggacacgcactggaaagtattctaggggcttcctgccttatccttctgtttgttcatacacttagtgaaagcagatcctgtactaggatgtgaggatatctgtcacctgcgtggtccattccataaacaccagattccatttcagttgccacacattttggggctagacttccctaaacaacatgccattgtgaaataatgcatatatttgtatttttaaatttccctgaaaatttaatctattctgttccatatgaagtctgtttcactaccttaaaaaatagatactccactagaggctgtgcttaattcaaatccatgtgtgtgcctgcattacatgtgtgaacacgtgtttctgtcgtgtgtgtcatgcacatctgtgtgttgcatagcataagccaaatgaaaatcatttgttcatgggattcatatgaggaacaaaattaaatttgaatacagtcagataactgccacgcagggcatttggggaaccatccccgaatgccctgatgtgatttccctcagaaaatccttgttattagaggagaaggtctgggcaggggcagcagcatctcagacatcaagtcaactttatcatctactacatcagcactgaagtccaggggcattgaggcactaactaggttccattttctttggtttggttacttattttttctttttgggttctcggtgtgcagaggctgtagagagagcctcagggcggtgaccgctgcaggaattcggccctccagctcccctccagttaccaggagccgatttggaggccaggccttcccagctgagtgtctgtcagcttcattctctctcagctgtgcagtggaagagcaacaacagacaggtgaccttcttgggttgtgcttcacaatggactgggaaaaacatctttgaaatcaggaaatcaggccgggcgcagtggctcatgcctgtaagcccagcactttgggaggcggaggcgggtggatccattgaggtcaggagctcaagaccagcctggtcaacatggtgaaaccccgtctctactaaaaatacaaaaaaaaaattagctgggcgtgttggcgggagcctgtagtcccagctacacgggaggctaaggtgggagaattgcttgaacccgggaggcggaggttgcagtgagctgagattgcaccattgcactccagcctgggcgacagagcaagactctctcaaaaaaaaaaaaaaaagaaagaaagaaatcagaaaatcgaccacagtggtagccacctggcctaatgctgtgtttttgtacctgacaggggtcactcattttaggcacaactccttcattctttgtgaaattagtgagtttccttctacccgtcaccagattcaatatgttctattaatacaccgataaccacaggggaagggcacttgtcgctctcccacctggttaccacagtctccatgggtcttttgccgtgaccacaaataaaggaaacactcatcactagtatctaagtcgggctttacagtaactatgcaccttctgtgtgcttcacctcactctctacttcaaacagcccatggagggaggtattattatactccttatgttgacagtgaagaatctgaggcccagagaggttggggacttgagtaaagtcacacagccctgagaggcaggaccagggttccattcctgctctatccagttccaagcccttgtgttttccattatgtttagtgcctctttgctaacagcaacatctgcaagatttgtgttggttttgatggagaactctagctcatccacatgctagtgcccaagtggtggaggggccacctcagcaggtgggttctgaatgcagccaaggctgtccccgcaatgggtgagactcgctccaactgcccgccctcagagcaggtgcctaagtcctccctggcactggcaggccttacctcacattgctaaattaaagcaatgcaattcctcttgggtaagaggaattcctccttctttactaactgatccccagcaaggaaataaaatgttaggctttaaaaatccctactttgtcatatcagactatattctaaaactatatttgagcgaaacctgtcattgcgtctaatttcaaatatacagaatctccttaagagctgttgccttatttttttgtaaagcctctctgacatcaaatggggagaaatggtggcacctccagacaccctgaaactacacaccatttcttccctgctcagcttctgctcaggagttctgtgagctatgggaaggccattggttgtatttgctacttttactttcatcttcctctgctgtagagccatttaatgttattgtcatatgctgctggtgaggtaaaggtgggtccgggtgccttcccaggggttagaggatgttcaaagggccgatttcagcaggagttcagagggcttatgatggatggtgagagatttgacaaccaccagagcacatgtgctctgaccctctcctgggcattggttcctgctggtaccgggcggttcagaccttcaaataggttgctttcaaaagagctttcaggcacttattgagaattaatgtttaaacagacataatagcctagatgaactcccaagagatctattaaatcttgtgggctgaataaatatctcgtgcaggactgtgcaacagtagcccagagcatcctgcctgtgggcatccacctcccaggtgagggcagtgggaagctggcccgacggcagccagaacttgtttctcacctcccaccagcaaccccccacccaactctgggccccaggcacacgaagcacaagtctcaggggaccattcccacattgggggatcctgagggagcccatcaccgcctcttgcatacaactgtccactaggaggcacgcccagtgtgggagagatgtatggtcttgccttccacctgtaaaaactgcacatatgcaagccatttgcactctggaactgcatgccgtgaaaactcctaatggtgtggaacttagtttgaatttgaaatcacgccgcatgcacaaagggacaggcccaggcccgacctcaggtcatccgcccgctggctgcagagcatccctgggagccaaggcgaggcccgtggagcctgagctttgtgtagctcgagctttgtgtagctcgtgcacttattatgcaccacctcccttcagtcaccactcctcttcctccgccatcctcatttatactgattgcacaccccccgctcaaacaacaatgtccttattatgatgaccatctcgtagtggtacattccattcctatttaaggtaagcccaaagcccacttttggattttctcgactgtccgagaaaagttgtgtaagcgcctgcgttcttctgggtttggctagatagggttgtgtccctctatggaatggagagtgatgtgggcaagggtgtcattttctcgcacaatacaactcactgaggatgcttctgtagaagtgagaaacacgatgagtacattcagaattacaataactcactctcactgggtaacttctcatgatagatttgtatgatcaatacgggtctatttttatgtcaactgaacactgtagggtaccttccagtctttttcaagattgttaaattgagacaagtaattgaataatttgtcctatttttattttaaaaaaagtgaatggactgaaatgttaaatgtgaatgtacatttcttaattgcaacttttctactgagtgtttgcactatactttctggaatcttatttaacaaaaataaagggaaaaaattgcttgactaaa

SEQ ID NO: 354 - DNA, 5811 bp, Homo sapiens
  source 1..5811: mol_type = other DNA
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 355 - DNA, 2226 bp, Homo sapiens
  source 1..2226: mol_type = other DNA
gtctgccggtcggttgtctggctgcgcgcgccacccgggcctctccagtgccccgcctggctcggcatccacccccagcccgactcacacgtgggttcccgcacgtccgccggccccccccgctgacgtcagcatagctgttccacttaaggcccctcccgcgcccagctcagagtgctgcagccgctgccgccgattccggatctcattgccacgcgcccccgacgaccgcccgacgtgcattcccgattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatctttaaattcatcattcctaatgttgtaaaatacagcccgaactgcaagttgcttattgtttcaaatccagtggatatcttgacctacgtggcttggaagataagtggttttcccaaaaaccgtgttattggaagcggttgcaatctggattcagcccgattccgttacctaatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtgactactttggttaattcattatattaagatataaagtcataaagctgctagttattatattaatttggaaatattaggctattcttgggcaaccctgcaacgattttttctaacagggatattattgactaatagcagaggatgtaatagtcaactgagttgtattggtaccacttccattgtaagtcccaaagtattatatatttgataataatgctaatcataattggaaagtaacattctatatgtaaatgtaaaatttatttgccaactgaatataggcaatgatagtgtgtcactatagggaacacagatttttgagatcttgtcctctggaagctggtaacaattaaaaacaatcttaaggcagggaaaaaaaaaaaaaaaaaa

SEQ ID NO: 356 - DNA, 1582 bp, Homo sapiens
  source 1..1582: mol_type = other DNA
agtctcacttcagttccttttgcatgaagagctcagaatcaaaagaggaaaccaacccctaagatgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtccagagaaaggtctggacatctatctcatcattggcatatgtggaggaggcagcctcttgatggtctttgtggcactgctcgttttctatatcaccaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaaaaaagatagaaactgtctttttcaataaaaagcactgtggatttctgccctcctgatgtgcatatccgtacttccatgaggtgttttctgtgtgcagaacattgtcacctcctgaggctgtgggccacagccacctctgcatcttcgaactcagccatgtggtcaacatctggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgcttaagagactctggagtttcttatgtgccctggtggacacttgcccaccatcctgtgagtaaaagtgaaataaaagctttgactagaaaaaaaaaaaaaaaaaa

SEQ ID NO: 357 - DNA, 3137 bp, Homo sapiens
  source 1..3137: mol_type = other DNA
agcaccgtctgggctgtggaagcggagggggtggggacactctggcccggttctcggtggtgcgggagcgggcgggagcagcggccgctctggtcggcggacgtgctgccgagtagtcccggaagcgaagcagcgatggcggagagtccgactgaggaggcggcaacggcgggcgccggggcggcgggccccggggcgagcagcgttgctggtgttgttggcgttagcggcagcggcggcgggttcgggccgcctttcctgccggatgtgtgggcggcggcggcggcagcgggcggggccgggggcccggggagcggcctggctccgctgcccgggctcccgccctcagccgctgcccacggggccgcgctgcttagccactgggaccccacgctcagctccgactgggacggcgagcgcaccgcgccgcagtgtctactccggatcaagcgggatatcatgtccatttataaggagcctcctccaggaatgttcgttgtacctgatactgttgacatgactaagattcatgcattgatcacaggcccatttgacactccttatgaagggggtttcttcctgttcgtgtttcggtgtccgcccgactatcccatccacccacctcgggtcaaactgatgacaacgggcaataacacagtgaggtttaaccccaacttctaccgcaatgggaaagtctgcttgagtattctaggtacatggactggacctgcctggagcccagcccagagcatctcctcagtgctcatctctatccagtccctgatgactgagaacccctatcacaatgagcccggctttgaacaggagagacatccaggagacagcaaaaactataatgaatgtatccggcacgagaccatcagagttgcagtctgtgacatgatggaaggaaagtgtccctgtcctgaacccctacgaggggtgatggagaagtcctttctggagtattacgacttctacgaggtggcctgcaaagatcgcctgcaccttcaaggccaaactatgcaggacccttttggagagaagcggggccactttgactaccagtccctcttgatgcgcctgggactgatacgtcagaaagtgctggagaggctccataatgagaatgcagaaatggactctgatagcagttcatctgggacagagacagaccttcatgggagcctgagggtttagaccctgctcccatctccccttcccccactcaagagtcccagcagaatcccttccccccaccccagggatggagaggcactgtgtatctccctccagactcgaagtcatcctgcaagatggcaagaaccaagcaagctccgatcccagggtgtgggagtgggggcctgttcccggtctgacctccttggcactggagcatctggggcttcgttcatccattcatcccgtatcaggggccaaggtacctttacaggagcacctagagcgagggcctttggcaaaaacaaaacaaccaacacacctctccacagggccagctccttagggataagtggaagatggaaattgcaattccaagagggagtgtgcccaaatgatttatggggatacctggaagggagcttggggtgggggctgtctgtgacacttaagcagtctgggtggttgtctatttgtctgtcttcagtcttgaagcagggcttcccaatgcccttttcctccctgccttccttcccccattatttcccacaggccagcataattttgtttttcctaatttatagtcactgttctagacagaccaaagagaaggaacagtggtggagtctaggctgctgatcagtaagctttacctagcacctgagcacctttctcccctcccctctttcctcacccttttctagatgtaagacagaaagtaaatgtgactgggacttaaccaaggtcttggtaaagcctgcatggcaccgtaagaagctgaaaatactgtttgttcccgcaatcactgatttgaaaagttcccaacacaggcagctgctgtgtatatgggattagagccactacatagaatagtctcttacagattttcataaatactagtcacaataagggtatttttcttgggggtggagtaagggggagactgatgctagtccttgttgtattttgttgggctgtccttgtgtattttcaccccagcctgtagtcctcctcacttcaaccccagggatttttggggagcaagggtagccaatggcagagggggttggggctgggactctggaggctcctccccttctttctcttccttccgcctcccccgtgcccccagctgctcttgtcactgtctctgatgggtatttgcctggctttgttgcttctctatctgtatttagctgcagtgatcctttagctggttggctcagaaaaaaaaaaatgtgctttaggtgccctgtaatcctgggcatcaagggaatccatccttcccctttttgatatgttctccccgtacttccagatttattgttatggctcccagtgggtattggcgattcttgtgatgcagggcctcagtcagtgtccagccatgcataagggagaggatagtgtgtacctgccctgccctctgctatgaaggtctctgccttgtggatcatgggactccccttggaggatctgtgcaaaggggggctgggcacaaaggagaatgtcctatttgggagggcaggaagcaaaggaactggacagggattggtgggcttggggaacggaagtttatcttggatacccttgaagaggctgggtctcttcacatgaagatcgaaaagggaccctgcttccaatttccctcttccattcctcgagctactccagggcttagaagaatgctcttggtctgtgggtccagtgttgtctgtcatccatttaagtgttcccactttcaagtgacaatcctctccttggccctgccatagggcagagcatgtctggcatagcagcctgacttttatgccctaatcttgagttgaggaaatatatgcacaggagtcaaagagatgtctttatatctgactgtatataaatgaagtttttttgttttttttgttttcctttttggtgcaataaagtttgttttggcagaaggagaaaaaaaaaaa

SEQ ID NO: 358 - DNA, 2759 bp, Homo sapiens
  source 1..2759: mol_type = other DNA
gtgcgtggcgtgcgtgcgttgacctgggaagcactggacctgtgaggcgtgcgaactggtggcagtgagagacttcggcggacatggctcccagcgtgccagcggcagaacccgagtatcctaaaggcatccgggccgtgctgctggggcctcccggggccggtaaagggacccaggcacccagattggctgaaaacttctgtgtctgccatttagctactggggacatgctgagggccatggtggcttctggctcagagctaggaaaaaagctgaaggcaactatggatgctgggaaactggtgagtgatgaaatggtagtggagctcattgagaagaatttggagacccccttgtgcaaaaatggttttcttctggatggcttccctcggactgtgaggcaggcagaaatgctcgatgacctcatggagaagaggaaagagaagcttgattctgtgattgaattcagcatcccagactctctgctgatccgaagaatcacaggaaggctgattcaccccaagagtggccgttcctaccacgaggagttcaaccctccaaaagagcccatgaaagatgacatcaccggggaacccttgatccgtcgatcagatgataatgaaaaggccttgaaaatccgcctgcaagcctaccacactcaaaccaccccactcatagagtactacaggaaacgggggatccactccgccatcgatgcatcccagacccccgatgtcgtgttcgcaagcatcctagcagccttctccaaagccacatgtaaagacttggttatgtttatctaatgttgggtccaagaaggaatttctttccatccctgtgaggcaatgggtgggaatgataggacaggcaaagagaagcttcctcaggctagcaaaaatatcatttgatgtattgattaaaaaagcacttgcttgatgtatctttggcgtgtgtgctactctcatctgtgtgtatgtgtgttgtgtgtgtgtgtgtgtgcatgcacatatgtgttcactctgctactttgtaagttttaggctaggttgctttaccagctgtttacttcttttttgttgttgttttgagacaaggtttcgctctgccaccctggctggagtgcagtggcgtgatcttggctcacggcaacctctgcctcctggggctcaagcaattatcccacctcagcctcctgagcagctgggactacaggtgcatgccacaacacctggctgatatttgtattttttgtagagacaggattttgccaagttgcccaggctggtcttgaactcctaggcttaagcaatccacccaccttggcctcctgaagtgccaggatcacagacgtgagccactacacccagcccagctgtttacttctttaaccatacttttgattttattttttgaccaaaatgaactaacccaggtaatcttccagggaccgcaattccagaacctcatagtatttcttccatttccagcagctgattagaagtccaggatcatgtgaagtcaggcagggtcacagttcctgatggcacattatggacagagaattccattttgttttctaacccatgatgaaaacccacgtgagtcagtgtgtgaacagggatcattaattttttccccctaggtggaaggaaaaaggcacttactttgcaggttacagaaattactgggagaggatatcgtcataaaaagagccaggccaaattggaatatttttgtgatctgcatcatgatgctgaaaatagcaattatttgggaattgggtttgaaaactgaattgttgccagagaattaaaccaggtgaaaggtccttttgaattcagattgtcttctgaacatccaggctgatcatctgagagcagtcaaatctacttccccaaaaagagaccagggtaggtttatttgcttttatttttaatgtttgcctgtgtttccaagtgtgaacaaaacagtgtgtgatctattcttggattcattttgatcagtatttattcaaacccagtctctctccaggacataaaactgaaatcagatatgttctttttaagcccaaaccctctcctttctagatccaacccttcacccctaattttatgatggctatagccatggacttccccaagaaaagatcacccagaaataagaccacctgtgacagttaccagcttttattcataaccttagcttcccaactattgagcattttctaaggtccctgctgtcttttggtctctggtttgatttgtggcaaacagatgaagtaacagactgctatgaaggaccacaaaaacggcagcctctggaaaaaccattagaaagtcagtggcagatccagtaaataatatcgccagcctcagcataatctgctgctgactcgattcagtggactctaaagtgcccagcctcctgacctgagctctcctgccatctgtgagactaccagaggtcttatctgctgtccacatggcaactgggcatgagtacctggccaccttgcttccctctttgcctggtccaagtgagtgtctgctgcctctgtcctgccttgttttcctggctctaaaccaactccacccactcttaatggaaactcagtctggctttgtgtgtttctgggaagcacatgacttctgggaatgggcaaggaagaggagtgaaacaaaaactgtcagctatgtgtgcctggtctgggatccttctctgggtgacagtggcatcatgaatcttagaatcagctcccc

SEQ ID NO: 359 - DNA, 2397 bp, Homo sapiens
  source 1..2397: mol_type = other DNA
cttaaacgcgactcaaggcgtcgggtttgttgtcaaccaatcacaaggcagcctcgctcgagcgcaggccaatcggctttctagctagagggtttaactcctatttaaaaagaagaacctttgaattctaacggctgagctcttggaagacttgggtccttgggtcgcaggtgggagccgacgggtgggtagaccgtgggggatatctcagtggcggacgaggacggcggggacaaggggcggctggtcggagtggcggagcgtcaagtcccctgtcggttcctccgtccctgagtgtccttggcgctgccttgtgcccgcccagcgcctttgcatccgctcctgggcaccgaggcgccctgtaggatactgcttgttacttattacagctagaggcatcatggaccgatctaaagaaaactgcatttcaggacctgttaaggctacagctccagttggaggtccaaaacgtgttctcgtgactcagcaatttccttgtcagaatccattacctgtaaatagtggccaggctcagcgggtcttgtgtccttcaaattcttcccagcgcattcctttgcaagcacaaaagcttgtctccagtcacaagccggttcagaatcagaagcagaagcaattgcaggcaaccagtgtacctcatcctgtctccaggccactgaataacacccaaaagagcaagcagcccctgccatcggcacctgaaaataatcctgaggaggaactggcatcaaaacagaaaaatgaagaatcaaaaaagaggcagtgggctttggaagactttgaaattggtcgccctctgggtaaaggaaagtttggtaatgtttatttggcaagagaaaagcaaagcaagtttattctggctcttaaagtgttatttaaagctcagctggagaaagccggagtggagcatcagctcagaagagaagtagaaatacagtcccaccttcggcatcctaatattcttagactgtatggttatttccatgatgctaccagagtctacctaattctggaatatgcaccacttggaacagtttatagagaacttcagaaactttcaaagtttgatgagcagagaactgctacttatataacagaattggcaaatgccctgtcttactgtcattcgaagagagttattcatagagacattaagccagagaacttacttcttggatcagctggagagcttaaaattgcagattttgggtggtcagtacatgctccatcttccaggaggaccactctctgtggcaccctggactacctgccccctgaaatgattgaaggtcggatgcatgatgagaaggtggatctctggagccttggagttctttgctatgaatttttagttgggaagcctccttttgaggcaaacacataccaagagacctacaaaagaatatcacgggttgaattcacattccctgactttgtaacagagggagccagggacctcatttcaagactgttgaagcataatcccagccagaggccaatgctcagagaagtacttgaacacccctggatcacagcaaattcatcaaaaccatcaaattgccaaaacaaagaatcagctagcaaacagtcttaggaatcgtgcagggggagaaatccttgagccagggctgccatataacctgacaggaacatgctactgaagtttattttaccattgactgctgccctcaatctagaacgctacacaagaaatatttgttttactcagcaggtgtgccttaacctccctattcagaaagctccacatcaataaacatgacactctgaagtgaaagtagccacgagaattgtgctacttatactggttcataatctggaggcaaggttcgactgcagccgccccgtcagcctgtgctaggcatggtgtcttcacaggaggcaaatccagagcctggctgtggggaaagtgaccactctgccctgaccccgatcagttaaggagctgtgcaataaccttcctagtacctgagtgagtgtgtaacttattgggttggcgaagcctggtaaagctgttggaatgagtatgtgattctttttaagtatgaaaataaagatatatgtacagacttgtattttttctctggtggcattcctttaggaatgctgtgtgtctgtccggcaccccggtaggcctgattgggtttctagtcctccttaaccacttatctcccatatgagagtgtgaaaaataggaacacgtgctctacctccatttagggatttgcttgggatacagaagaggccatgtgtctcagagctgttaagggcttatttttttaaaacattggagtcatagcatgtgtgtaaactttaaatatgcaaataaataagtatctatgtctaaaaaaaaaaaaaaaa

SEQ ID NO: 360 - DNA, 1278 bp, Homo sapiens
  source 1..1278: mol_type = other DNA
ccattggcctgtagattcacctcccctgggcagggccccaggacccaggataatatctgtgcctcctgcccagaaccctccaagcagacacaatggtaagaatggtgcctgtcctgctgtctctgctgctgcttctgggtcctgctgtcccccaggagaaccaagatggtcgttactctctgacctatatctacactgggctgtccaagcatgttgaagacgtccccgcgtttcaggcccttggctcactcaatgacctccagttctttagatacaacagtaaagacaggaagtctcagcccatgggactctggagacaggtggaaggaatggaggattggaagcaggacagccaacttcagaaggccagggaggacatctttatggagaccctgaaagacatcgtggagtattacaacgacagtaacgggtctcacgtattgcagggaaggtttggttgtgagatcgagaataacagaagcagcggagcattctggaaatattactatgatggaaaggactacattgaattcaacaaagaaatcccagcctgggtccccttcgacccagcagcccagataaccaagcagaagtgggaggcagaaccagtctacgtgcagcgggccaaggcttacctggaggaggagtgccctgcgactctgcggaaatacctgaaatacagcaaaaatatcctggaccggcaagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagcccacccccaccccattgtctaatctgtagaagctaataaataatcatccctccttgcctagcataaaaaaaaaaaaaaa

SEQ ID NO: 361 - DNA, 13037 bp, Homo sapiens
  source 1..13037: mol_type = other DNA
agtccacagctgtcactaatcggggtaagccttgttgtatttgtgcgtgtgggtggcattctcaatgagaactagcttcacttgtcatttgagtgaaatctacaacccgaggcggctagtgctcccgcactactgggatctgagatcttcggagatgactgtcgcccgcagtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcgggagataaaggagccgcgtgtcactaaattgccgtcgcagccgcagccactcaagtgccggacttgtgagtactctgcgtctccagtcctcggacagaagttggagaactctcttggagaactccccgagttaggagacgagatctcctaacaattactactttttcttgcgctccccacttgccgctcgctgggacaaacgacagccacagttcccctgacgacaggatggaggccaagggcaggagctgaccagcgccgccctcccccgcccccgacccaggaggtggagatccctccggtccagccacattcaacacccactttctcctccctctgcccctatattcccgaaaccccctcctccttcccttttccctcctcctggagacgggggaggagaaaaggggagtccagtcgtcatgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtgaatgtcatctttttcttttaaagaattaaattttgtggtatgtctttttgttttggtcaggattatgaggtcttgagtttttataatgttcttctgaaagccttacatttataacatcatagtgtgtaaatttaaaagaaaaattgtgaggttctaattattttcttttataaagtataattagaatgtttaactgttttgtttacccatattttcttgaagaatttacaagattgaaaaagtactaaaattgttaaagtaaactatcttatccatattatttcataccatgtaggtgaggatttttaacttttgcatctaacaaatcatcgacttaagagaaaaaatcttacatgtaataacacaaagctattatatgttatttctaggtaactccctttgtgtcaattatatttccaaaaatgaacctttaaaatggtatgcaaaattttgtctatatatatttgtgtgaggaggaaattcataactttcctcagattttcaaaagtatttttaatgcaaaaaatgtagaaagagtttaaaaccactaaaatagattgatgttcttcaaactaggcaaaacaactcatatgttaagaccattttccagattggaaacacaaatctcttaggaagttaataagtagattcatatcattatgcaaatagtattgtgggttttgtaggtttttaaaataaccttttttggggagagaattgtcctctaatgaggtattgcgagtggacataagaaatcagaagattatggcctaactgtactccttaccaactgtggcatgctgaaagttagtcactcttactgattctcaattctctcacctttgaaagtagtaaaatatctttcctgccaattgctcctttgggtcagagcttattaacatcttttcaaatcaaaggaaagaagaaagggagaggaggaggagggaggtatcaattcacatacctttctcctctttatcctccactatcatgaattcatattatgtttcagccatgcaaatctttttaccatgaaatttcttccagaattttccccctttgacacaaattccatgcatgtttcaaccttcgagactcagccaaatgtcatttctgtaaaatcttccctgagtcttccaagcagtaatttgccttctcctagagtttacctgccattttgtgcacatttgagttacagtagcatgttattttacaattgtgactctcctgggagtctgggagccatataaagtggtcaatagtgtttgctgactgagagttgaatgacattttctctctgtcttggtattactgtagatttcgatcattctttggttacatttctgcatatttctgtacccatgactttatcactttcttctcccatgctttatctccatcaattatcttcattacttttaaattttccacctttgcttcctactttgtgagatctctccctttactgactataacatagaagaatagaagtgtattttatgtgtcttaaggacaatactttagattccttgttctaagtttttaaactgaatgaatggaatattatttctctccctaagcaaaattccacaaaacaattatttcttatgtttatgtagccttaaattgttttgtactgtaaacctcagcataaaaactttcttcatttctaatttcattcaacaaatattgattgaatacctggtattagcacaagaaaaatgtgctaataagccttatgagaatttggagctgaagaaagacatataactcaggaaagttacagtccagtagtaggtataaattacagtgcctgataaataggcattttaatatttgtacactcaacgtatactaggtaggtgcaaaacatttacatataattttactgatacccatgcagcacaaaggtactaactttaaatattaaataacacctttatgtgtcagtaattcatttgcattaaatcttattgaaaaggctttcaatatattttccccacaaatgtcatcccaagaaaaaagtatttttaacatctcccaaatataatagttacaggaaatctacctctgtgagagtgacacctctcagaatgaactgtgtgacacaagaaaatgaatgtaggtctatccaaaaaaaaccccaagaaacaaaaacaatattattagccctttatgcttaagtgatggactcagggaacagttgatgttgtgatcattttattatctgattcttgttactttgaattaaaccaatattttgatgatataaatcatttccaccagcatatatttaatttccataataactttaaaattttctaatttcactcaactatgagggaatagaatgtggtggccacaggtttggcttttgttaaaatgtttgatatcttcgatgttgatctctgtctgcaatgtagatgtctaaacactaggatttaatatttaaggctaagctttaaaaataaagtacctttttaaaaagaatatggcttcaccaaatggaaaatacctaatttctaaatctttttctctacaaagtcctatctactaatgtctccattactatttagtcatcataaccattatcttcattttacatgtcgtgttctttctggtagctctaaaatgacactaaatcataagaagacaggttacatatcaggaaatacttgaaggttactgaaatagattcttgagttaatgaaaatattttctgtaaaaaggtttgaaaagccatttgagtctaaagcattatacctccattatcagtagttatgtgacaattgtgtgtgtgtttaatgtttaaagatgtggcactttttaataaggcaatgctatgctattttttcccatttaacattaagataatttattgctatacagatgatatggaaatatgatgaacaatattttttttgccaaaactatgccttgtaagtagccatggaatgtcaacctgtaacttaaattatccacagatagtcatgtgtttgatgatgggcactgtggagataactgacataggactgtgccccccttctctgccacttactagctggatgagattaagcaagtcatttaactgctctgattaaacctgcctttcccaagtgctttgtaatgaatagaaatggaaaccaaaaaaaacgtatacaggccttcagaaatagtaattgctactattttgttttcattaagccatagttctggctataattttatcaaactcaccagctatattctacagtgaaagcaggattctagaaagtctcactgttttatttatgtcaccatgtgctatgatatatttggttgaattcatttgaaattagggctggaagtattcaagtaatttcttctgctgaaaaaatacagtgttttgagtttagggcctgttttatcaaagttctaaagagcctatcactcttccattgtagacattttaaaataatgacactgattttaacatttttaagtgtctttttagaacagagagcctgactagaacacagcccctccaaaaacccatgctcaaattatttttactatggcagcaattccacaaaagggaacaatgggtttagaaattacaatgaagtcatcaacccaaaaaacatccctatccctaagaaggttatgatataaaatgcccacaagaaatctatgtctgctttaatctgtcttttattgctttggaaggatggctattacatttttagtttttgctgtgaatacctgagcagtttctctcatccatacttatccttcacacatcagaagtcaggatagaatatgaatcattttaaaaacttttacaactccagagccatgtgcataagaagcattcaaaacttgccaaaacatacattttttttcaaatttaaagatactctatttttgtattcaatagctcaacaactgtggtccccactgataaagtgaagtggacaaggagacaagtaatggcataagtttgtttttcccaaagtatgcctgttcaatagccattggatgtgggaaatttctacatctcttaaaattttacagaaaatacatagccagatagtctagcaaaagttcaccaagtcctaaattgcttatccttacttcactaagtcatgaaatcattttaatgaaaagaacatcacctaggttttgtggtttctttttttcttattcatggctgagtgaaaacaacaatctctgtttctccctagcatctgtggactatttaatgtaccattattccacactctatggtccttactaaatacaaaattgaacaaaaagcagtaaaacaactgactcttcacccatattataaaatataatccaagccagattagtcaacatccataagatgaatccaagctgaactgggcctagattattgagttcaggttggatcacatccctatttattaataaacttaggaaagaaggccttacagaccatcagttagctggagctaatagaacctacacttctaaagttcggcctagaatcaatgtggccttaaaagctgaaaagaagcaggaaagaacagttttcttcaataatttgtccaccctgtcactggagaaaatttaagaatttgggggtgttggtagtaagttaaacacagcagctgttcatggcagaaattattcaatacataccttctctgaatatcctataaccaaagcaaagaaaaacaccaaggggtttgttctcctccttggagttgacctcattccaaggcagagctcaggtcacaggcacaggggctgcgcccaagcttgtccgcagccttatgcagctgtggagtctggaagactgttgcaggactgctggcctagtcccagaatgtcagcctcattttcgatttactggctcttgttgctgtatgtcatgctgaccttattgttaaacacaggtttgtttgctttttttccactcatggagacatgggagaggcattatttttaagctggttgaaagctttaaccgataaagcatttttagagaaatgtgaatcaggcagctaagaaagcatactctgtccattacggtaaagaaaatgcacagattattaactctgcagtgtggcattagtgtcctggtcaatattcggatagatatgaataaaatatttaaatggtattgtaaatagttttcaggacatatgctatagcttatttttattatcttttgaaattgctcttaatacatcaaatcctgatgtattcaatttatcagatataaattattctaaatgaagcccagttaaatgtttttgtcttgtcagttatatgttaagtttctgatctctttgtctatgacgtttactaatctgcatttttactgttatgaattattttagacagcagtggtttcaagctttttgccactaaaaataccttttattttctcctcccccagaaaagtctataccttgaagtatctatccaccaaactgtacttctattaagaaatagttattgtgttttcttaatgttttgttattcaaagacatatcaatgaaagctgctgagcagcatgaataacaattatatccacacagatttgatatattttgtgcagccttaacttgatagtataaaatgtcattgctttttaaataatagttagtcaatggacttctatcatagctttcctaaactaggttaagatccagagctttggggtcataatatattacatacaattaagttatctttttctaagggctttaaaattcatgagaataaccaaaaaaggtatgtggagagttaatacaaacataccatattcttgttgaaacagagatgtggctctgcttgttctccataaggtagaaatactttccagaatttgcctaaactagtaagccctgaatttgctatgattagggataggaagagattttcacatggcagactttagaattcttcactttagccagtaaagtatctccttttgatcttagtattctgtgtattttaacttttctgagttgtgcatgtttataagaaaaatcagcacaaagggtttaagttaaagcctttttactgaaatttgaaagaaacagaagaaaatatcaaagttctttgtattttgagaggattaaatatgatttacaaaagttacatggagggctctctaaaacattaaattaattattttttgttgaaaagtcttactttaggcatcattttattcctcagcaactagctgtgaagcctttactgtgctgtatgccagtcactctgctagattgtggagattaccagtgttcccgtcttctccgagcttagagttggatggggaataaagacaggtaaacagatagctacaatattgtactgtgaatgcttatgctggaggaagtacagggaactattggagcacctaagaggagcacctaccttgaatttaggggttagcagaggcatcctgaaaaaagtcaaagctaagccacaatctataagcagtttaggaattagcagaacgtgcgtggtgaggagatgccaaaggcaagaagagaagagtattccaaacaggagggattccaaagagagaagagtatcccaaacaacatttgcacaaacctgatggggagagagaatgtggggtggggatggatgatgagactgaagaagaaagccaggtctagataatcagtggccttgtacaccatgttaaagagtgtagacttgattctgttgtaaacaggaaagcagcacaattcatatgaatattttagaagactcccactggaatatggagaataaagttggagatgactaatcctggaagcagggagaacatttttgaggaagttgcactattttggtgaaaatgatgatcataaacatgaagaattgtaggtgatcatgacctcctctctaattttccagaagggttttggaagatataacataggaacattgacaggactgacgaaaggagatgaaatacaccatataaattgtcaaacacaaggccagatgtctaattattttgcttatgtgttgaaattacaaatttttcatcaggaaaccaaaaactacaaaacttagttttcccaagtcccagaattctatctgtccaaacaatctgtaccactccacctatatccctacctttgcatgtctgtccaacctcaaagtccaggtctatacacacgggtaagactagagcagttcaagtttcagaaaatgagaaagaggaactgagttgtgctgaacccatacaaaataaacacattctttgtatagattcttggaacctcgagaggaattcacctaactcataggtatttgatggtatgaatccatggctgggctcggcttttaaaaagccttatctgggattccttctatggaaccaagttccatcaaagcccatttaaaagcctacattaaaaacaaaattcttgctgcattgtatacaaataatgatgtcatgatcaaataatcagatgccattatcaagtggaattacaaaatggtatacccactccaaaaaaaaaaaaaaagctaaattctcagtagaacattgtgacttcatgagccctccacagccttggagctgaggagggagcactggtgagcagtaggttgaagagaaaacttggcgcttaataatctatccatgttttttcatctaaaagagccttctttttggattaccttattcaatttccatcaaggaaattgttagttccactaaccagacagcagctgggaaggcagaagcttactgtatgtacatggtagctgtgggaaggaggtttctttctccaggtcctcactggccatacaccagtcccttgttagttatgcctggtcatagacccccgttgctatcatctcatatttaagtctttggcttgtgaatttatctattctttcagcttcagcactgcagagtgctgggactttgctaacttccatttcttgctggcttagcacattcctcataggcccagctcttttctcatctggccctgctgtggagtcaccttgccccttcaggagagccatggcttaccactgcctgctaagcctccactcagctgccaccacactaaatccaagcttctctaagatgttgcagactttacaggcaagcataaaaggcttgatcttcctggacttccctttacttgtctgaatctcacctccttcaactttcagtctcagaatgtaggcatttgtcctctttgccctacatcttccttcttctgaatcatgaaagcctctcacttcctcttgctatgtgctggaggcttctgtcaggttttagaatgagttctcatctagtcctagtagcttttgatgcttaagtccaccttttaaggatacctttgagatttagaccatgtttttcgcttgagaaagccctaatctccagacttgcctttctgtggatttcaaagaccaactgaggaagtcaaaagctgaatgttgactttctttgaacatttccgctataacaattccaattctcctcagagcaatatgcctgcctccaactgaccaggagaaaggtccagtgccaaagagaaaaacacaaagattaattatttcagttgagcacatactttcaaagtggtttgggtattcatatgaggttttctgtcaagagggtgagactcttcatctatccatgtgtgcctgacagttctcctggcactggctggtaacagatgcaaaactgtaaaaattaagtgatcatgtattttaacgatatcatcacatacttattttctatgtaatgttttaaatttcccctaacatactttgactgttttgcacatggtagatattcacatttttttgtgttgaagttgatgcaatcttcaaagttatctaccccgttgcttattagtaaaactagtgttaatacttggcaagagatgcagggaatctttctcatgactcacgccctatttagttattaatgctactaccctattttgagtaagtagtaggtccctaagtacattgtccagagttatacttttaaagatatttagccccatatacttcttgaatctaaagtcatacaccttgctcctcatttctgagtgggaaagacatttgagagtatgttgacaattgttctgaaggtttttgccaagaaggtgaaactgtcctttcatctgtgtatgcctggggctgggtccctggcagtgatggggtgacaatgcaaagctgtaaaaactaggtgctagtgggcacctaatatcatcatcatatacttattttcaagctaatatgcaaaatcccatctctgtttttaaactaagtgtagatttcagagaaaatattttgtggttcacataagaaaacagtctactcagcttgacaagtgttttatgttaaattggctggtggtttgaaatgaatcatcttcacataatgttttctttaaaaatattgtgaatttaactctaattcttgttattctgtgtgataataaagaataaactaatttcta

SEQ ID NO: 362 - DNA, 3190 bp, Homo sapiens
  source 1..3190: mol_type = other DNA
cacagcacatccctttctcctcttttcactcatcgtcaccgctacctgaaaaccctggccgggtgctggggcttgaggagcagttcccacttcccagtctttttcacttttcacagctgcaaagttcagggagttgaactgcagtgctttcagttcactgctcactctgccacgatcaatctctgttgtaaattttcctcccagagcacgtgacgatgcacttcttgactatatatcccaactgcagcagcggagttgtcagagcgcagagccggacagagcagaagaaccctcttggactggacgatttgggaattcaaaacttgggacaaactgtcagccttgcccctgctgtggaggcagcctcaatgctgaaaatggagcctctgaacagcacgcaccccggcaccgccgcctccagcagccccctggagtcccgtgcggccggcggcggcagcggcaatggcaacgagtacttctacattctggttgtcatgtccttctacggcattttcttgatcggaatcatgctgggctacatgaaatccaagaggcgggagaagaagtccagcctcctgctgctgtacaaagacgaggagcggctctggggggaggccatgaagccgctgcctgtggtgtcgggcctgaggtcggtgcaggtgcccctgatgctgaacatgctgcaggagagcgtggcgcccgcgctgtcctgcaccctctgttccatggaaggggacagcgtgagctccgagtcctcctccccggacgtgcacctcaccattcaggaggagggggcagacgatgagctggaggagacctcggagacgcccctcaacgagagcagcgaagggtcctcggagaacatccatcagaattcctagcacccccgggacccctgccggtggctccatcagccagcaaccttagagagaggaaagacagttttcaagtgtctggtttcactttcacagtgcggctgccactttgaagagacccttggtaaacccctgattcggggtggggtgggggactaggctcagccggaaccagcacctccaaggagtccgggaggtgcctgtggtttgcacccaccactgaaaaagccgcggagatgcgcagcgcgtacactgactttggggcctgggtgttggggttctgatcagaatttggcgggatgatatgtttgccattttctcactggatgccctgggtagctcctgcagggtctgcctgttcccagggctgccgaatgcttaggacacgctgagagactagttgtgatttgctattttgcctagagctttgtccttctagatctgattggctgtaagtatctctactgtgtacctgtggcattccttcacagtgggttacaagcttcttttggattagagggggatttttgatgggagaaagctggagatctgaacccagcccatttgcacactataagaaaaaaaagtaacttttaaacctgttaacattggccggggttataagagatgatcttctattttgaccttttgtctaacttatgaccttgaactctgacctgtgaccatgcagcatcacatgatggcatgacgttctttggatcagaagagcttccccagaatctaacctgcactcccgatggtggttcaggagactcttcctgatctttctagaaggggtaaagtggggttgaacaaggccaagccgttagctctgctgctgctcagtttcccagcctagttttctgagctgggagaggacataatgtggtatttcgtcacgtagctgagacctagaagggcatactcaggcggagattgcacaaagctgggctccaagttctgtgcttcctgatcccgagccctgacactcatttgctgtgtggtcccgggcatgtcatcaagaagctcgctgtctgcatgaggctcccgacgatctccatccccagggggttgggaggatgtctttagattttaggactctgtgataaatattccacagcctggtgtgaggaagcttggaccaaatgcttcccctttggccggttagtctgaacaaggatctgctgatttaaaggagcagttggagattcagaacacatataactggggaattcagggcaagctaccaacccagccctgctgtagttttcccaggactgaaggaaagaggcagacaccacattctcctctgcaaagttctctggagaatgtgtgatgttaacactcccagctaagggaaagcaaagtctaaaggaaaacacacaggaatgcttccaatgtctccaagtattcctgtggtcaacagtttcgcagatccgttaggcccttaaagacaaggaggaagtaaaaggtcaaatttaaatctatttaaaaaaacttttaaaaaaattatttatttattattattttttatttttaagacagaatctcgctctgttgcccaggctggagtgcagtggtgcaatcttggctcactgcaacctccacctcctgggtttcagcgattctcctgcctcagcctcctgagtagctgggattacaggcgcctgccaccacgcccagctaattttttgatttttagtagagatggggtttcgccattttggccaggctggtctcaaactcctggccttaaatgatcctcccacctcggcctcccaaagtgctgggattacaggcatgagccactgcacccggccactttttttttttttaaagaaaaatgctctgcatggattggagacacagcaataactactgttgccatggaagggttaacagtgtaggagctggtttatcagtccgctttgacatacagctaaaggaaatttatgtttgggggaaaaaggccctctgttcactttaaaattcagtgtggacttatgccaaagggggctgtttaagttgaaagaagccaagttaagtttggcctcttgcctggaatcacttgaattctgaaatttcactgcgacggacatgtgccttgtcacattttccattgcttaatcctgaagtttggtgcaagtctctctgcacctattaaaaagtgatgtatatacttccttcttattctgttgagttgtatagaattgtcttttgtatttaacactttgtaattttcacaatattttttaatttaaataaataaacacattttttccctcctgggaagtcatgaaaaaaaaaaaaaaaaa

SEQ ID NO: 363 - DNA, 6499 bp, Homo sapiens
  source 1..6499: mol_type = other DNA
acacgcagccccgcccctgcagagatcagcgctgggacggaacccgggttcctctcgaaccgggattgtgacgcttttggcctggctggccgctgttttctgtcccactttttactcgggcctgcgtccgctgccgccgtccctcagtttgcccccggaggaggcagggcggccgtgccttctgccgtgcgcccgcgtggctgccaccgcccctccgaatcctccggggccgcagaggggttcgctacggagggaggtgggggccttcgggaggaggaggcggaggaggcggaggaggagggaaggaagatggcggccgtggaactagagtggatcccagagactctctataacaccgccatctccgctgtcgtggacaactacatccgctcccgccgagacatccgctccttgcccgagaacatccagtttgatgtttactacaagctttaccaacagggacgcttatgtcaactgggcagtgaattttgtgaattggaagtttttgctaaagtactgagagctttggataaaagacatttgcttcatcattgttttcaggctttgatggatcatggtgttaaagttgcttcagtcttggcctactcattcagtaggcggtgctcttatatagcagaatcagatgctgcagtaaaggaaaaagccattcaggttggctttgttttaggtggctttctttcagatgcaggctggtacagtgatgctgagaaagtttttctgtcctgccttcagttgtgtactctacacgatgagatgcttcattggtttcgtgcagtagaatgttgtgtgaggttgcttcatgtgcgaaatggaaactgcaaatatcatttgggtgaagaaacatttaaattagctcagacatatatggataaactatcaaaacatggccagcaagcaaataaagctgcactctatggagaactgtgtgcactcctatttgcaaaaagtcactatgatgaggcatacaaatggtgcatcgaggcaatgaaagaaattacagcaggcttaccagtgaaagttgtggtggatgtcttaagacaagcttctaaggcttgtgtagtaaaacgtgaatttaagaaggcagaacagttaattaaacatgcagtgtatttggcacgggatcattttggatccaaacacccaaaatattctgatacactgctagattatgggttctacttactcaatgtagataatatctgtcagtctgttgcaatttatcaggcagcccttgacattagacagtcagtgtttggtggcaaaaatatccacgtagcaacagctcatgaagatttggcctactcttcttatgtccaccagtatagctctgggaaatttgacaatgcactatttcatgcagaaagagctattggtatcattacccacatcctacctgaagatcatcttcttttggcttcttcaaagagggtgaaagcacttattttagaggagattgcaattgattgtcataataaggaaactgaacagaggctgcttcaagaagctcatgatttgcacctgtcttcactccaactagctaaaaaagcttttggggaatttaatgtacagactgcaaaacactatggaaaccttggaagactttatcagtcaatgagaaaatttaaggaagctgaagaaatgcacatcaaagcaattcagattaaagaacaacttcttggtcaagaagattatgaagtagccctttcagtgggacatctggcttctttatataattatgacatgaatcagtatgaaaatgctgagaaactttatttgcgatctatagcaattgggaagaaactttttggtgagggctacagtggactagaatatgattatcgaggtctcattaaactttacaactccattggaaattacgagaaagtgtttgaatatcacaatgttctgtctaactggaaccggttgcgagatcggcaatattcagtgacagatgctcttgaagatgtcagcaccagcccccagtccactgaagaagtggtgcagtccttcctgatttctcagaatgtcgagggaccgagctgctgagggaggacctcagttaaccaattaccttttcccggattccagggaattcatactgtgaaatcaaaaccatgttgttttggggggctggaatttgcattgaaacactggtccagtccattgaagaccctattttgggtgatccctatcttgcagaatgtctgtaggaataagcatatattcagttatattcagcatgtaccgcatgtgtaagtagtctggcccacattttcaacctagtagaacaaacaacaggaaatcttttttttgttgtttttaaaaaattcattttgcagaaagcctgaaagaaaaaaaatacccctaaataaaactatttaagagtttaaaagagttgcattcttattatgtaaggatgattttaacaactttttaatatgtaattcttccatgtggaggtattcaatactgtagtgtaaagaaattttatgcggaaaatctttatatgcagtatagaaaagttaacacaagtactaataaaagagggacatcccgacttacgtttttctaccttgcccagataagtggatacaaccactctatattacaaggaaaggactgtcagattcatctgaactggaccagtgttgatctgtaatgtaatagaaaatctgatagaccagcacttctgacttttttttttttggtacaacaatgcaagatgctctgatagcatttgctaacaggaccaggaggatctaaaaaggaccagcctaatgtagaaggtggttatttggaccagaggctttagattattattttagatcctacatatacttttatcagtagaatgatttcatttagatgtataatgaaaaaggataatgcaaaaattatgtaatagataccaaattagggaagtttggcaatttcaatggcatatttttagtcaaggtacacagatggcagtgccataagcaagtctataaatatcggctgcagccatccccctcattttaaatgttgccctaataatcaatgcagttaacaagtatattggctgtgtgtcatgaaatagttcatgttcagatggaaatgttaggttactgtatggtttatggagattaatgaaaatgaatgcccaaaaataagtcttagaaaatcctccatttttatggtaaatagtaatacaactaggtcatttcatttgaaatctaggagtcaaatggaaagatcccctaataatacacctatttcactaacttgtctttctgtttattgggttttgatttgattttttgtaagccagtcaggttatttaatgatgaggtaataatcaaatttaagaatttgtgacatgtagcaattcaagaaacaaaaaggtattttgctgttacctcaattcttactgtagtagcccatctgatgcttctatagttaagaatctgggttccccccctattttcaggggttcatgacttggctgttaaagatgttgctcctagctaatgcttggagtagtctgtgggtgaatggatgtgtgttgaattttagttttcttttaacatgcatgttgggtgagaggggaaaaaaatctaagctgtctgccacattgagtacagaaaagttgtagatttcaaattttattaatattttaagcacttttttgaacttcccaaccttgtttgaagctgttatttgcagtcctattagttttgagccattgcatttaagttccctaggagggggttggttgggggatgtactgaaagagatgaagcaaacccacaccctaagatggtaactgtgtgatttagaaacctgagtttactcctcaaatcgaattattttctttttaaattttggaaagagtaaattgacgtacttgcagtttatgaagctgccccccacccctcagttaattgcagtctaatgtcaagaggcacttctttattaattaccaaatagtctttgtgaccaaggactaacatttttaagttactcagctctatcctcatgggcctatatatttaatacctccaaagatattttcaggataggctttgtatacttttattggttatttagaatccagtggtatgtttgtggtataggaatgtcatggtaaattgtttttcaataaatattttgaaacatgtttccatatgaagtttttttttcaatctgtattttttggttttgtgcacatacagcatttcctaggataaaaataaacaaatgacttacagcctcatcctccctaactccatttgaactcaacttagctcacactcagtgataaaacaacatggtatgtagaagcctaggatcacagggtgataatgtcaattggcagccagttgtgtttttttgaaacatcattattggcagtttctccttatcaccactgctttaatgtagtttttttgtaaatccatatactttaatgcatacactctagcttaagaaaacattgccattttggttagggatatgacttaatgtgctattatttctggttctaatgaagaataataccctatgactttaagtgtaagattcatcctttaagtagggatgtttaggataagttagatgtgtgccactatgatttattgggtttcttaaaaatcttgaagaaaataataaaatttatctcacaataagttaacttgcgcaaactttttacatatggtgaggtgcgtaaggaagccctggccaacttaaagatttttctggaggttcagcaaagttatgttaaattaggggcctttggtctcatccttctctgactcttctacccagtcttttcctaaagttcggtgctactccagttgggtgcatcagggagctccgtcagcactcgcatgtgtcgctcaggtggccactcatgcctgcattccatttaatagagtcaattggaatttttagagcataatctttatggatcctcaaagctgactttgccaaagggattgagacccttactaccatcaaatctctgtctctgcttggttaaaaattggctcactattgctttgtagtaacccctgcccaggtattttttcacttgtgaaaataatttgagaaagacctttgttcctagcctgttgggaaaagtttataattttatgaaaattaagtacagaggctgcgatcttagaaataatgaaggtgccatttggctgctccttaatagtgcagacagaaaactgcagtgaacacatgccaaaacatgattgaagcctttggctgaaactttatacatagaaataatgatttgctcataacaggtatcattaactgccactttttatgttttccctagaatttgtagccttgctgcttgcttttcttctgggtggcaaagttactactggaaaaacactataagtacaaagtttttggggttttatctttgctttagaagtggatgtgtacttcacctcttggctgtggaggaacttagttgccaggaaattttttttttttttttcagacggagtttcgctcttgttgcccaggctgggagggcaatggcaccgtcttggctcactgcaacctccgcctcccgggttccagcgattctcctgcctcagccttccgagtagctgggattacaggctcctgccaccacgcctggctaggaaattttttgttgttaatatgacatttggattaatctccagcttcaacagtacttcttttgtccataaatctcaggaatgttttaggcagaaaactggttttaccctgttgataatcagaaggagtgtgctttaggatttattgcataatactattctttaattgcaatcctaggtatctatagcatgagtggccttagtgagtttgttgaagtgcacatgtttttcaagagtaaaatttaagattaaaaatatatcctatatatagatatctagaaaacttggtttgtggtgcacagttaagtgttggatcactaaataaccattgcaggtaccgtttgtgtaacattactcatttctgtatattccttttatgggaagatattttgccatggtaactaaaacttttcagttctacttttatgatgtgaatgaatgctacgttttattaaatattaccaggtcagtactatttttatactttattaagcaacaggggattttagtttaataggctcaaaataaaaagttcttaatggaacagttaaaaacaaaacactaacaatctttacgtgaaaatccccactaatagtgccacaataatttctatagaaatatctaaggtcattaaatagatttttgaagacggttcttcattgtgtcaggatgacctttcatatcattctcaccaacttgtagtgcccaccgttatttgtaactattaaaccatactaagtatgtttgtaaccagcattgtgatatattctgtacttgtattgctaaaaatgaattattgacctaataaatatagtgttcctgcattcatagtg

SEQ ID NO: 364 - DNA, 771 bp, Homo sapiens
  source 1..771: mol_type = other DNA
agagaagcagacatcttctagttcctcccccactctcctctttccggtacctgtgagtcagctaggggagggcagctctcacccaggctgatagttcggtgacctggctttatctactggatgagttccgctgggagatggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtgaacctgagactggtggcttctagaagcagccattaccaactgtaccttcccttcttgctcagccaataaatatatcctctttcactcagaaaaaaaaaaaaaaaaaaaaaaaaaaaaaa

SEQ ID NO: 365 - DNA, 1218 bp, Homo sapiens
  source 1..1218: mol_type = other DNA
aaaggtctaggatgacatctggtgtattgactgtggccagtcttaaagctagtttttgctatgtggaacatgctgctctaattcagatttaaagagtttcttcctgttaattcgaagctcactgtgcctcttgtttccgagggaagaaggactgattaagtcatctaaatggatgcaatactgaattacaggtcagaagatactgaagattactacacattactgggatgtgatgaactatcttcggttgaacaaatcctggcagaatttaaagtcagagctctggaatgtcacccagacaagcatcctgaaaaccccaaagctgtggagacttttcagaaactgcagaaggcaaaggagattctgaccaatgaagagagtcgagcccgctatgaccactggcgaaggagccagatgtcgatgccattccagcagtgggaagctttgaatgactcagtgaagacgtcaatgcactgggttgtcagaggtaaaaaagacctgatgctggaagaatctgacaagactcataccaccaagatggaaaatgaggaatgtaatgagcaaagagaaagaaagaaagaggagctggcttcaaccgcagagaaaacggagcagaaagaacccaagcccctagagaagtcagtctccccgcaaaattcagattcttcaggttttgcagatgtgaatggttggcaccttcgtttccgctggtccaaggatgctccctcagaactcctgaggaagttcagaaactatgaaatatgaaatatctctgcttcaaaaaatgaggaagagcaagactgtcccctatgctgccaacatgcagtctttgtttatgtcttaaaaatgtcatgtttatgtcatgtctgtgaattgctgagtactaattgattcctccatccttgaatcagttctcataatgctttttaaataagaaaaattcagaagatgaatttcttccaatatttgaataaattaaagctcttagatacagagtagattgtattatatgctttttcctattaatactacttatagaaatccattaaaaagcaatctctgtacagtgtatttaaatatttcattgacatactgtgatctctattagtgatggatgtacaaaaaatgttttcttacccttgacttacaatgaaatgtgaaattacttgtctgaaccccgtggggagaaataaataattttcccaaagttcaaaaaaa

SEQ ID NO: 366 - DNA, 2974 bp, Homo sapiens
  source 1..2974: mol_type = other DNA
gtctgtgtcgaataatgtgtggcttctgttggattgcttttctttccaaaattcctaggcaatgcttccccgaggtgtgcacctttgtgaggtgtttgtggggttgggggagcttcaggcgctactcgcgggacgccgtcacgtgatccgggatgaggtggagttcggctttaaggaggcgtctcttcctagcttcatcaatctttaggatctgagcaggagaaataccagcggatcttccccactctgctcccttccattcccacccttccttctttaataagcaggagcgaaaaagacaaattccaaagaggattgttcagttcaagggaatgaagaattcagaataattttggtaaatggattccaatatggggaataagaataagctgaacagttgacctgctttgaagaaacatactgtccatttgtctaaaataatctataacaaccaaaccaatcaaaatgaattcaacattattttcccaggttgaaaatcattcagtccactctaatttctcagagaagaatgcccagcttctggcttttgaaaatgatgattgtcatctgcccttggccatgatatttaccttagctcttgcttatggagctgtgatcattcttggtgtctctggaaacctggccttgatcataatcatcttgaaacaaaaggagatgagaaatgttaccaacatcctgattgtgaacctttccttctcagacttgcttgttgccatcatgtgtctcccctttacatttgtctacacattaatggaccactgggtctttggtgaggcgatgtgtaagttgaatccttttgtgcaatgtgtttcaatcactgtgtccattttctctctggttctcattgctgtggaacgacatcagctgataatcaaccctcgagggtggagaccaaataatagacatgcttatgtaggtattgctgtgatttgggtccttgctgtggcttcttctttgcctttcctgatctaccaagtaatgactgatgagccgttccaaaatgtaacacttgatgcgtacaaagacaaatacgtgtgctttgatcaatttccatcggactctcataggttgtcttataccactctcctcttggtgctgcagtattttggtccactttgttttatatttatttgctacttcaagatatatatacgcctaaaaaggagaaacaacatgatggacaagatgagagacaataagtacaggtccagtgaaaccaaaagaatcaatatcatgctgctctccattgtggtagcatttgcagtctgctggctccctcttaccatctttaacactgtgtttgattggaatcatcagatcattgctacctgcaaccacaatctgttattcctgctctgccacctcacagcaatgatatccacttgtgtcaaccccatattttatgggttcctgaacaaaaacttccagagagacttgcagttcttcttcaacttttgtgatttccggtctcgggatgatgattatgaaacaatagccatgtccacgatgcacacagatgtttccaaaacttctttgaagcaagcaagcccagtcgcatttaaaaaaatcaacaacaatgatgataatgaaaaaatctgaaactacttatagcctatggtcccggatgacatctgtttaaaaacaagcacaacctgcaacatactttgattacctgttctcccaaggaatggggttgaaatcatttgaaaatgactaagattttcttgtcttgctttttactgcttttgttgtagttgtcataattacatttggaacaaaaggtgtgggctttggggtcttctggaaatagttttgaccagacatctttgaagtgctttttgtgaatttatgcatataatataaagacttttatactgtacttattggaatgaaatttctttaaagtattactattaactgacttcagaagtacctgccatccaatacggtcattagattgggtcatcttgattagattagattagattagattgtcaacagattgggccatccttactttatgataggcatcattttagtgtgttacaatagtaacagtatgcaaaagcagcattcaggagccgaaagatagtctgaagtcattcagaagtggtttgaggtttctgttttttggtggtttttgtttgttttttttttttttcaccttaagggaggatttaatttgctcccaactgattgtcacttaaatgaaaatttaaaaatgaataaaaagacatacttctcagctgcaaatattatggagaattggggcacccacaggaatgaagagagaaagcagctccctaacttcaaaaccattttggtacctgacaacaagagcattttagagtaattaatttaataaagtaaattagtattgctgcaaatagttaaattatatttatttgaattgatggtcaagagattttccattttttttacagactgttcagtgtttgtcaagctttctggcataaatatgtactcaaaaggcatttccgcttacaatttgtagaaacacaaaatgcgttttccatacagcagtgcctatatagtgactgatttttaactttcaatgtccatctttcaaaggaagtaacaccaaggtacaatgttaaaggaatattcactttacctagcagggaaaaatacacaaaaactgcagatacttcatatagcccattttaacttgtataaactgtgtgacttgtggcgtcttataaataatgcactgtaaagattactgaatagttgtgtcatgttaatgtgcctaatttcatgtatcttgtaatcatgattgagcctcagaatcatttggagaaactatattttaaagaacaagacatacttcaatgtattatacagataaagtattacatgtgtttgattttaaaagggcggacattttattaaaatcaatattgtttttgctttttcaaaaaaaaaaaaaaaa

SEQ ID NO: 367 - DNA, 5586 bp, Homo sapiens
  source 1..5586: mol_type = other DNA
tcttgtgatcaccctcattacctcttctgggccccctgtggacctgcgttgacccagcatgggctacaatgggggagttgggtaatcgctcccggtaggttcctccccaacccccgaggcggggtcaggtggagcgtgggcgccagagcagcccctctgccttgggtgtcttggagatgacttggcgccccccggagccccgtcatggctgtggctgcgtccacaacactgcatcagcagagtcctggggaagccatttaggaatctgaagcccgggatttctgctttgtcccttcaggggttcctgagcctgttaaaaaaaaaaaaaaatctaggaagaggccctggaaggaaaaaggggagggagatagcctattttacaaggtctgaaatgatgtgtcttatccttctggcttaaaccaagctgggcttaaaacatcttttaaaaaaaatctgtattctatttttttctctctgtttatcgctctccttctgcctctgttaatctttgtttctgtgtagttgcatcctgtttctgtctgaaaacccttgtctttctgaatccatctttcagtctctcacattctgtctttcccctcctccttcaccttccctccgtccctctcccttgcgcacttgccgggtaccagctgagtgcatccttttcccaaacctgctctgcttccaaggggcccttttctgtctcctccacctgtccccatgttttggggactgcctggaaatccgggacaccctcctgaggagggagctggtgcccaacggcacctcttaggatgaacccagccagctgcgttcctggcctctgcagaatggcatacagaaggcattttaagcaagcgagctgagctcgccgcaggactccgactaccaggcaccggtggcagcacgcaaagggtgtccctgtccctcaaggggtcatggcctccatgttgctcgcccagcgcctggcctgcagcttccagcacagctaccgcctgctggtgcctggatccagacacattagtcaagctgcagccaaagtcgacgttgaatttgattatgatgggcctctgatgaagacggaagtcccagggcctagatctcaggagttaatgaaacagctgaatataattcagaatgcagaggctgtgcattttttctgcaattacgaagagagccgaggcaattacctggttgatgtggacggcaaccgaatgctggatctttattcccagatctcctctgtccccataggttacagccaccccgccctgctgaaactcatccaacagcctcaaaatgcgagcatgtttgtcaacagacccgccctcggaatcctgcctccggagaactttgtggagaagctccggcagtccttgctctcggtggctcccaaagggatgtcccagctcatcaccatggcctgcggctcctgctccaatgaaaacgccttaaagaccatcttcatgtggtaccggagcaaggaaagagggcagaggggcttctcccaggaggagctggagacgtgcatgattaaccaggcccctggctgccccgactacagcatcctctccttcatgggcgcgttccatgggaggaccatgggttgcttagcgaccacgcactctaaagccattcacaagatcgacatcccttcctttgactggcccatcgcaccgttcccacggctgaaataccctctggaagagtttgtgaaagagaaccaacaggaggaggcccgctgtctggaagaggtggaggatctgattgtgaaatatcggaaaaagaagaagacggtggccgggatcatcgtggagcccatccagtccgagggtggagacaaccacgcatccgatgacttctttcggaagctgagagacatcgccaggaagcatggctgcgccttcttggtggacgaggtccagaccggaggaggctgcacgggcaagttctgggcccatgagcactggggcctggatgacccagcagacgtgatgaccttcagcaagaagatgatgactgggggcttcttccacaaggaggagttcaggcctaatgctccctaccggatcttcaacacctggctgggggacccgtccaagaacctgttgctggctgaggtcatcaacatcatcaagcgggaggacctgctaaataatgcagcccatgccgggaaggccctgctcacaggactgctggacctccaggcccggtacccccagttcatcagcagggtgagaggacgaggcaccttttgctccttcgatactcccgatgattccatacggaataagctcattttaattgccagaaacaaaggtgtggtgttgggtggctgtggtgacaaatccattcgtttccgtcccacgctggtcttcagggatcaccacgctcacctgttcctcaatattttcagtgacatcttagcagacttcaagtaaagaagccatttccactacagtgagaaagcccggatcccaacagttgtcaaattgattagtttgcctaattcatgttttcacttaaaagtatcagaggtgaatgcacagtgaagggtgatttgtggggagggagcatttttggtggtcttgggggaggggaggggagggaagggctggtgttgattttcctccctgcagagccaatggtgcacattggtttaagcccagagatcctgcttgagccctggactcatcttgggaagggccatgggaggtcctggctagagttctgcccaaccttgaccaaccccagcaatttttccaaaagccagtcaagggcattacatttgttcctgggactggcagctgggcctcctgggtgccagtccatctcaagtgccatattctgtgatcacggatgttggctccccctcgccctatgcaagcaaacacactctcacctcctctcccagcctcccggagctctgagcacgccccacgcatggtgcaggagggactggacagatctgaggaaggccgtaaaatggggacaggaggattcagctcaggagtaagggaagaggtcctctggccccctgaagatgagaatcacccctctcagtgcacctggggatccccctaaactgtgaattctgccctggacagggctttgcccttggaggatccccaggtggccttacccgtgattcttactcggcatcctccattccccaaactccctcctccctgttaaatgtcaacctagacctggacctgggtgggaggaaactgtagcctgagtgtccacagggacacacgtgagccacagggctagaagcacaggcccgtcaccccaggaggaaaagcccagggtctggggcagcagagccatcgcatcatgtttcccagggcaacttgaggtctgacttttggctccctcgccctaagaggctcttcctctcttttgctcatccaccttcacaccacttgtgagctgacccccaggaacagcttggctgttccaccagagcagagggaatcattgctggactggatttcttctggagggaagcaccatgaattaaaggtgccagagctccatgcatggaaaagtggcctgttggttcaacgtggtttccttgtacttgatcctcctgaaatgagagattttcctgagatgactgagtatggagattaccaggcagatgataccgaaatgcttaaaggggctgtggctgaggctgtagcatctctgctggaggtgagacactctgggaactgatttgacctcgaatgctcctaaaagagaacttgatagcctgacagcagagaagtattatttggtggggagtctgagaaagcctggcttgtattatctgtccaaaaggaagcctcttcatctcccggtgccttggttgactattttggaaataggcatatcaaggttcctgattcagcactttgctcttttaataacaaatcgtccaaagatctcaaagtaagggtttttttttttagcttccactcttccgcagacttggtcatcgacctatttttgttgaagaatgaaaggcaatgacaagtttaacatatacaaattcgtgaattcttgttcattttgcattcctgcagtcagtgctaatccgtcgttcttaatcaggagttctgccaggtttaaggtcacctgcactcccagccaagtggtgatgtatgcctcccactctctgcaaggttttcctcatttttatattcatttattcatcattcattcattcacctaactttcattaaatagctgcaccatcccaggccctgtgcggagcatcgggaatacaaagatgagataaaacattgtccctgcccccgggggctcacatgccactggaaacaacagactcttaaactaacaattcaagcaggcgccaagtgctatgacagaggtgtgaataaaagcatcaggaaatcggatggagaaccaccattcacccaagggtcagaggaggtttcaaagaaaaggtgtcattggagcagtgttttgcagaatgagccagagttcaccaagcaggcaaggcaaggaagggtgttgcagagagggaacagcatatgcaaaggcgtatcagttcatggcatcacatctttcactggggaagccagtttccacaggcagcatcccaaggttcaataaggccttatttaacaagcaagcaaaatgcaaaccaaaccattattcatttattggcttaagtatgctttctcctgaaaactttagcattgggtgcaaatattcagtatggttctcggagtccaaagggttttaagccagggcacaaccagaaaagtggctctctttggtagggagaggggctccaatatttcgttctctccccatggggcactgacagagaaatgaaatagttttatctggaaaattccagagctattatttactccttaccaagggaagttacttcttgtaaaaacttttaagccatttatcaacaagttcttgttgactcagggcataatgagttcctgagacatgctcttttgggggctggggctttagctagaagaatttcaaggaaaagaattctcagcagagctcaagattgtagaaactcagcagaagctggtaaaaacatggggagcccggaggacaggctgctgtccagggcagaggccatgaagaagtgcttccgtggccgacagtctggaaatgaatccatcatacattagtgccatagagtttagtaaccgtccagcaagtgtcatcacttttacagaaaacaaggtccagtaatagcaagtcttagtacatcctcacttttattataaatgggtgttttttcataatttttactgacgctcagtaaccatgcaaaattgtgtatagcattaatgtatctacatacctacacctatctatatataagctcatggtagaaaaccatagctaagtagcatcgcagacttaagcgtacaaagtgatcttgttcacaagtaatctgttgacagtgccaataaatgataaaaaaaaaattaacatgtcacaatgtaacggatgaccatatgcacaattccatgaattaaatctgtttcctgtgttagtcagtattcttaaataaaatttataattgaaacatga

SEQ ID NO: 368 - DNA, 2185 bp, Homo sapiens
  source 1..2185: mol_type = other DNA
tggttttaccttttccgggagtctccagctggccctcatttgtgtccggagctcaggagttcccaaaccgactcagtcgcaccaagtttccgtcttttggaattggggaaggagtttctttctttcttttcttttttcttgagccagttttaatcgctttgaataaatactcccttaagtagttaaatataggaggagaaagaatacatcggttgttaaagcaggagaggaagagagacctgccctgtagcgtgactcctctagaaaaaaaaaaaaaaagccggagtattttactaagcccctaaaatgtcgagatttgtacaagatcttagcaaagcaatgtctcaagatggtgcttctcagttccaagaagtcattcggcaagagctagaattatctgtgaagaaggaactagaaaaaatactcaccacagcatcatcacatgaatttgagcacaccaaaaaagacctggatggatttcggaagctatttcatagatttttgcaagaaaaggggccttctgtggattggggaaaaatccagagaccccctgaagattcgattcaaccctatgaaaagataaaggccaggggcttgcctgataatatatcttccgtgttgaacaaactagtggtggtgaaactcaatggtggtttgggaaccagcatgggctgcaaaggccctaaaagtctgattggtgtgaggaatgagaatacctttctggatctgactgttcagcaaattgaacatttgaataaaacctacaatacagatgttcctcttgttttaatgaactcttttaacacggatgaagataccaaaaaaatactacagaagtacaatcattgtcgtgtgaaaatctacactttcaatcaaagcaggtacccgaggattaataaagaatctttacttcctgtagcaaaggacgtgtcttactcaggggaaaatacagaagcttggtaccctccaggtcatggtgatatttacgccagtttctacaactctggattgcttgatacctttataggagaaggcaaagagtatatttttgtgtctaacatagataatctgggtgccacagtggatctgtatattcttaatcatctaatgaacccacccaatggaaaacgctgtgaatttgtcatggaagtcacaaataaaacacgtgcagatgtaaagggcgggacactcactcaatatgaaggcaaactgagactggtggaaattgctcaagtgccaaaagcacatgtagacgagttcaagtctgtatcaaagttcaaaatatttaatacaaacaacctatggatttctcttgcagcagttaaaagactgcaggagcaaaatgccattgacatggaaatcattgtgaatgcaaagactttggatggaggcctgaatgtcattcaattagaaactgcagtaggggctgccatcaaaagttttgagaattctctaggtattaatgtgccaaggagccgttttctgcctgtcaaaaccacatcagatctcttgctggtgatgtcaaacctctatagtcttaatgcaggatctctgacaatgagtgaaaagcgggaatttcctacagtgcccttggttaaattaggcagttcttttacgaaggttcaagattatctaagaagatttgaaagtataccagatatgcttgaattggatcacctcacagtttcaggagatgtgacatttggaaaaaatgtttcattaaagggaacggttatcatcattgcaaatcatggtgacagaattgatatcccacctggagcagtattagagaacaagattgtgtctggaaaccttcgcatcttggaccactgaaatgaaaaatactgtggacacttaaataatgggctagtttcttacaatgaaatgttctctaggattctaaaataggcaggtactttactatgttactgtaccctgcagtgttgatttttaaaatagagttttctgcagtatgcttttagtctaagaaaagcacagatggagcaatactttccttctttgaagagaatcccaaaagttagttcatcttaaagtgcaatattgtttaatcttaaaactgggcaactttggaagaacttttaacagaagcctcaatgatgatcactttgaattgcttgtgatttcaaaaataaagcagtgaagcaataaaaaaaaaaaaaaaaaa

SEQ ID NO: 369 - DNA, 528 bp, Homo sapiens
  source 1..528: mol_type = other DNA
gtgcattccctacccagcagccctcgcgcggtccggcacagcggacaccaggactccaaaatggcgtcagttggtgagtgtccggccccagtaccagtgaaggacaagaaacttctggaggtcaaactgggggagctgccaagctggatcttgatgcgggacttcagtcctagtggcattttcggagcgtttcaaagaggttactaccggtactacaacaagtacatcaatgtgaagaaggggagcatctcggggattaccatggtgctggcatgctacgtgctctttagctactccttttcctacaagcatctcaagcacgagcggctccgcaaataccactgaagaggacacactctgcacccccccaccccacgaccttggcccgagcccctccgtgaggaacacaatctcaatcgttgctgaatcctttcatatcctaataggaattaacctccaaataaaacatgactggtacgtgtgctgattgcctgcctgtctgccgtagccaaaaaaaaaaaaaaaaaa

SEQ ID NO: 370 - DNA, 1660 bp, Homo sapiens
  source 1..1660: mol_type = other DNA
acagtccctccgggtggcggggcgccccgagcgtggcagcgcgctaggcggcagcagcgggcgcggagcggggcgcggccgccgcgcgttccctcttggcggggttggccggccggggcggggcgcggcgctccggctcgaggcattcggagctgcgggagccgggctggcaggagcaggatggcggcggcggcggctgcaggcgaggcgcgccgggtgctggtgtacggcggcaggggcgctctgggttctcgatgcgtgcaggcttttcgggcccgcaactggtgggttgccagcgttgatgtggtggagaatgaagaggccagcgctagcatcattgttaaaatgacagactcgttcactgagcaggctgaccaggtgactgctgaggttggaaagctcttgggtgaagagaaggtggatgcaattctttgcgttgctggaggatgggccgggggcaatgccaaatccaagtctctctttaagaactgtgacctgatgtggaagcagagcatatggacatcgaccatctccagccatctggctaccaagcatctcaaggaaggaggcctcctgaccttggctggcgcaaaggctgccctggatgggactcctggtatgatcgggtacggcatggccaagggtgctgttcaccagctctgccagagcctggctgggaagaacagcggcatgccgcccggggcagccgccatcgctgtgctcccggttaccctggataccccgatgaacaggaaatcaatgcctgaggctgacttcagctcctggacacccttagaattcctagttgaaactttccatgactggatcacagggaaaaaccgaccgagctcaggaagcctaatccaggtggtaaccacagaaggaaggacggaactcaccccagcatatttttaggcctcatctcagtgcctatgaggggcctgccagaaaagtcactaacctgtctcagtgtggccttgtccagccttgtgttttctgtaacccctgtttgtggtacgagataatgagtcctatttttctctcacataatatgcatttgctctcctaggacagtgtaatacatttatgtgaagtaaagacatgcgagactggtggcctgcaaatagcatccgttgatctgtgttaactgcatagggagggctctgcatagcacctgctatagcggtgtcatgttggatcgcttttgtgactgttcatctgtccttgacagtggctgtcatcttgactactttgttgatttgttggtattggggacattttaaaggctgagttatttttgaatgtcatgtttatgtcatagacgtagttttcgcatccttgaattaaactgccttaactccttttgtggtataagcaaaactacatggactctgtcctggtatccttttcctgtgtggttgccccgtgtcctctggcctagggttaagtgtgcaagataactactcgtgagtattcagaatgttgttcctaataaatgcacttgttgtctgtcttctttaatcaaatcacatcttatatacagcagtcagagatgagtatactagaatcatggattgctggaggtcttttaatctgatgttctcagaagggggtggatttaaatcctgaaataaatatttcaacacaagaacaaaaaaaa

SEQ ID NO: 371 - DNA, 1825 bp, Homo sapiens
  source 1..1825: mol_type = other DNA
ggcagtcccaaaaagcctctcaggcacacaacccagcacagcaaggcagtcacaccaaggcggtccccagcgcgctgcctccgccgttccccgccccctcgcctctgaggctgcccggatagctgcctgatccaaggcccagcagcttctgccttccggatccccaccgacggacggaggcagcccacctgcaggaccggggcgctgtcggtccacgtggccgctctggagcaggttggagttggcagcgggctgcgctgcagggacaggcgcggcggacagccccgggggcagcggcggggtcctgtctgggagaggacagggttgcggcgggcggaacggtgtctccttcacttcgccctccagctgctggagctgcagcccgaccgcgagcgtgccaagcggcttcagcagctagcggagcggtggcggcggcccccctcaggacaccaccagattcccctcttcccgcggcctcgccatggcgacccacggacagacttgcgcgcgtccaatgtgtattcctccatcatatgctgaccttggcaaagctgccagagatattttcaacaaaggatttggttttgggttggtgaaactggatgtgaaaacaaagtcttgcagtggcgtggaattttcaacgtccggttcatctaatacagacactggtaaagttactgggaccttggagaccaaatacaagtggtgtgagtatggtctgactttcacagaaaagtggaacactgataacactctgggaacagaaatcgcaattgaagaccagatttgtcaaggtttgaaactgacatttgatactaccttctcaccaaacacaggaaagaaaagtggtaaaatcaagtcttcttacaagagggagtgtataaaccttggttgtgatgttgactttgattttgctggacctgcaatccatggttcagctgtctttggttatgagggctggcttgctggctaccagatgacctttgacagtgccaaatcaaagctgacaaggaataactttgcagtgggctacaggactggggacttccagctacacactaatgtcaatgatgggacagaatttggaggatcaatttatcagaaagtttgtgaagatcttgacacttcagtaaaccttgcttggacatcaggtaccaactgcactcgttttggcattgcagctaaatatcagttggatcccactgcttccatttctgcaaaagtcaacaactctagcttaattggagtaggctatactcagactctgaggcctggtgtgaagcttacactctctgctctggtagatgggaagagcattaatgctggaggccacaaggttgggctcgccctggagttggaggcttaatccagctgaaagaaacctttgggaatggatatcagaagatttggccttaatatatttccattgtgaccagcagcaggcttttttcccccaagaagatgatcaaaacaaaggatgatctcaacaagagctgtattttaagtatttagacagttctttgttagctggtttctagttggttatctagttaccaatgctgcagtcctgcagtcacctatacattatttaaatgtatttaactgttaaatgcgctacccaccaataatgaaatagacctttatgaaaactgtgcaattgtgtgcatgtttgtttttatgttcctttagaaaacattgactgttaccattgaatgagatggatcagtggatattaagatgaggttacaaattttgttaagttcagccattattacttttggtatcccagaacatgacaaattatgaataaaacaagtatacataa

SEQ ID NO: 372 - DNA, 7096 bp, Homo sapiens
  source 1..7096: mol_type = other DNA
gagaccctgcgcgcggccgccagcgctccgggatctcgagatagccgcagctctcgcgatctttctggagccgcacctccacgcggagtccgagcgcgtgtgctgagaccccagggtcgggagggcggagactgggagggagggagaagcccctttggcctgccttacggaagcctgcgagggagggtggtgtccactgcccagttccgtgtcccgatgcccagcgccagcgccagccgcaagagtcaggagaagccgcgggagatcatggacgcggcggaagattatgctaaagagagatatggaatatcttcaatgatacaatcacaagaaaaaccagatcgagttttggttcgggttagagacttgacaatacaaaaagctgatgaagttgtttgggtacgtgcaagagttcatacaagcagagctaaagggaaacagtgcttcttagtcctacgtcagcagcagtttaatgtccaggctcttgtggcggtgggagaccatgcaagcaagcagatggttaaatttgctgccaacatcaacaaagagagcattgtggatgtagaaggtgttgtgagaaaagtgaatcagaaaattggaagctgtacacagcaagacgttgagttacatgttcagaagatttatgtgatcagtttggctgaaccccgtctgcccctgcagctggatgatgctgttcggcctgaggcagaaggagaagaggaaggaagagctactgttaaccaggatacaagattagacaacagagtcattgatcttaggacatcaactagtcaggcagtcttccgtctccagtctggcatctgccatctcttccgagaaactttaattaacaaaggttttgtggaaatccaaactcctaaaattatttcagctgccagtgaaggaggagccaatgtttttactgtgtcatattttaaaaataatgcatacctggctcagtccccacagctatataagcaaatgtgcatttgtgctgattttgagaaggttttctctattggaccagtattcagagcggaagactctaatacccatagacatctaactgagtttgttggtttggacattgaaatggcttttaattaccattaccacgaagttatggaagaaattgctgacaccatggtacaaatattcaaaggacttcaagaaaggtttcagactgaaattcaaacagtgaataaacagttcccatgtgagccattcaaatttttggagccaactctaagactagaatattgtgaagcattggctatgcttagggaagctggagtcgaaatgggagatgaagacgatctgagcacaccaaatgaaaagctgttgggtcatttggtaaaggaaaagtatgatacagatttttatattcttgataaatatccattggctgtaagacctttctataccatgcctgacccaagaaatcccaaacagtccaactcttacgatatgttcatgagaggagaagaaatattgtcaggagctcaaagaatacatgatcctcaactgctaacagagagagctttacatcatggaattgatttggagaaaattaaggcttacattgattccttccgctttggagcccctcctcatgctggtggaggcattggattggaacgagttactatgctgtttctgggattgcataatgttcgtcagacctccatgttccctcgtgatcccaaacgactcactccttaaattcacactttgccacttaactccagtgtggatgacagagcgagaccctgcctcaaaaaaaaaaaaaaaaaaagaaagccacacttattcttttcagtaacctgctagtgcacaggctgtactttaggtacttaaaatatgcactagaataaatttgcaaggccctaaaatatcactgttatttttggagtaattcagtataggttcgtttaaaagagatttttataacttcagacatgcatcagtaggaaataacttgagaaattcatatggttatgttacaaattcatattctgttactacagtaaacgttaagagttttaaacagttaagattgtacaatttttcttcttttctatattacaagggccccagtgttaatgtcttagattttcagtatttgaacttatttttttaaattctgtcattgagataagaataattcaggtagcatctgaaattttaatgaatgtataattggcatatcatggaaaattaaccagaaagtatcagttcttaaaagttatgcctagaaattatgtaaagctaaactactggttagaaagtattcagtgtaatattgtattaatttgttaaattctaaacttgaatttcaataaaattttaaagctaattgtagtttttgtgtaactttgtggccaactgattttgtaagtggtgctaggcattcataatttggtaaaagaaaaaatacacaactagtttatataaataaagactaatatgttgtcaacatgtattcatttcaggatattagtttagatgggcgtagggatagagctcttaaagttgacagcttgggtcggttgacagagttcatttcacatgggttaaaatatattgcagaattttgtttgatttttaactggaatggtactgatacttagtgaatgtcagtaaaatccagtcccatcttatccgtcaaaaagaaatttttgtgtaagaattagttagaaacactttccctctagattattacaaatgttgatgctaatgttaattttacatgaccacaaatattggggaggaacaaaacataacatatttttgtacaggaagaaaaaattacatgcagaaaaatatgagggtcaaaaagttaggaagttacccctggaccacgaacatctgcccattatgtttgaaagttaaagggggtgtttaccatctatattgtacagtctttgaaagtttagattcagttcttatttttttctttcatatcctaccagaataccttaaatatagaaggcaccccaaaagtatttcataatttaatccacagcaagaataaaatatccatcttctatagtgtgtaattgctagaccctaaaatagtacaataatattggccctagagctgttcttaatttcatgtaattatagcttgtttcaaaataaagtttgtaaagtcttaaaaaaaaaaaaaaaccttctcttcttctcagatggtcccttccattcccctgaaacctgcatgagagctcctaacatgtttctccaatgcaatcaagccctagactccaaatgtcctcccagctcacctccatctatgcatctcatctctggatttggtgatcagactctatattgacagtaggatctcaaaccctgcatccatccttcctccagcaagccctgctagccacatgaggaacaagtttccgtgtcttctgccttcctcttggggaaaggtgccttgttgtgatgaattaactcactgttagggcagggtggagaatggtactccttccttctcctgtccactgtgggggaagcttggcaggtatattatatttcatcatttaggaggctggcatgaccaggacttatgggtgggaggggagcatttttagtgaagcaagaaaggagtttgccaagaagtgatctgttttaaaggtcatatttggagaaagggcaaggaattgggtctgctttatttttgggggtattttgtttttgttctcacctgctgcccccccaccccaccaccccagggataaattggatataaacactaaatactaatcagttgaacttaacatttaataaaaagaaagggtgaaataaactgaagaccattttagaactagtcagttctctgcagcaaagggaacaggagccatttgaaccctctgggacccctcaccccactgcttcagggtgctaggctgagggatgtttttcctcccccttaccgcccatgcccttgaaagaaaagtcactttttgtggagggcatcattcattcctgattcacaaaccccaaaaacctctggtgggagataggaagatagggcgtgggcctgggccttaacctcaatcttgtgtctgcctcagtcttttctgactggccctgaagttgtcagtggctctttctgtccttcagcccctggaaggtgctccaggataacaaagaagggcaggttgaagcccctcatggaaggagctggctttgtggggctgcaaaggacttttaagtcctgcctgtactgaagttcacagcccacctgactgagcagactcttcctgttcctttctctaccacccttgccttcccaggactgcacggtttaacacagcagagtacagaagggtgaagaagtgagcagaggcttatgaagatattcagatactcttctatgccaggaagcacaaagactttgttgagatttgcctcagttcagtagatcttccttggcagccagccataggttgtttctttgtcttccgggtcctaaagagcacagagaaaatggaggtccccagtctaggtaggaagctgattggatgaggacttctttttttccgacagcaggatggggctcttgggctccacacaccagatgctttggttttctacaactgttgctatgtgtagagggtgctcagagcgtggcatgagagcaaggagaccatggctactctttgaaatggatggggaaaattagcttaaaaatttaatcacgagattgcgccactgcactccagcctgggcgacagagccagactccgtctcaaaaaaaaaaaaaaaaaaaaaatttaatcacacacatcacaggcttaaactgtcttagagacctttccagttgattggatcacaggctagggatctgtgcatggtgccaggtgaaacccaagtctccccagtgctaggtgagggagggctcaacgggggtaatgtgtatggggtctgctgtcctttgccatcccctggaccatattaactctgggacagaccatgattaggtctgtcaccaaacttttcctaatcattttttatgtatcattcttctttctaatctgttagcagttatctcccagaagtatccctagcatcctgaagcaaacagaactgccgggcgctgtggcttatgcctgtaatctcagcaatttgggaggccgaggcgggcggatcacctgaggtcagaagttcgagaccagcctgtccaacatggtgaaaccccgtctccactaaaaacacaaaaattatctgggcgtggtggcatgtgcctgtagtcccagctactggggaggctgaggcaggagaatcgcttgaatccaggaggtggaggttgcagtgagtggagatggcgccactgcgctccagcccgggtgacagagtgaaactccatctcagaaaacaaacaaaccaaacaactgagagagggcatgctagtggagggaaaaggagagagtacaagagttgatactgcatcagagttccagggcagatactgacaagtgactgaactttgactgtgaagtcttcccatttatttttgaaatgggagtcgatgccttttgtacgttatcaaagtgtttttttttttttcctcctttactcagacatagagcatcaaaatatcacgagtaaagccaaaccccagcttttcactggggctgatatcttcctccccatggcccactgccccctcatattccccagcacttggactgtgtcacatgggtattgattgtatacttgttgtcttggcctcatgcaaaaaggcctgggtctagatctagtcagatgagttttttttagagcatgttgatgatacaacacctgtttaaatgtctgccttatatgttatttggtcccactgttaataatagatttattactatgtatatttactattgaagatgggaatgtgatctgcatagttatcagtctattttgtatgttgtaaaaagcttctaatttaggtttaaggtgggctggctacaagagcactaaaactcctcaccttttaaactgctctttttatctgcttgtgggaatgtcgtctctttcgtggaagattgggtggtctcatgttgaggctgttgcccagtcccattaactcccttgtccccccacagaaggaagagacattgcccagctaagcatcaggaagctgtgttaaaagcccttctatgggtttggttttgtgatgtttttccctaatgggaaaaacgttatagttgtttcttactgccctgtctgggaagcagggcaaacctccaggtttttaaatgagctagatgcccctcttcctcttctctggtcactgaacctggaccaaagcactttgatattccaggtgtgattttctctgtcatggggatttgctccactgcagagccccatcattttcacagcgtaggccaacagagtgagaacctagggctaccctagctgatggatgtgaggctgctgtctacaggagctcatcccagccctgttaactggcagtggcaaggatactcgtcatcggccattgcactggggaactccctcaccccatggcttcccaacttgaaacccagatttacctccagggagaggtgagaaaaaaattgtaaatagacttgctaaagagcaactcagggttggggtgtgttttaattctcctgatcacttgaaataatctgtaggctgagtgcttatgggggtgggggagaagggtgactccagggtccttccattttgtgaagctctgggggtggagtgtgggcatctgaggccctatgatggcactacattgagctgtctgcccttccggaaacccaacctgcaatcaactgcaaatcaaattcttcacattccagctacagtctttctttccccattgaatctcagtccctggccatgtggtcaaggtggctttctgttaagctaccctaatttcgggaatgggaggggagagaggagggccattacaactctgccttcaagactcatctcttaaaaacaaaacgaaacaaaactacaaccaccatcaaaaccacacgcaaaaaaaaaaaaaaaaaaaaaaaggataactttaaccgaaggaagggtttggttccattcaactccacattcattg

SEQ ID NO: 373 - DNA, 176 bp, Homo sapiens
  source 1..176: mol_type = other DNA
gcagttgtccaagtatgaatctcaactttcaacaaatgaggagaaagtagatacagatgatcgaactgaaggctatttacgagcagactcacaagagccctcccactttgattctcagcagcctgcagtcttggaagaagaagaggtcatgatagctcatgctcatccacaggaag

SEQ ID NO: 374 - DNA, 194 bp, Homo sapiens
  source 1..194: mol_type = other DNA
agttcatgaccctggctttggtgttggccacctttgacccggcgcgggggaccgacgccaccaacccacccgagggtccccaagacaggagctcccagcagaaaggccgcctgtccctgcagaatacagcggagatccagcactgtttggtcaacgctggcgatgtggggtgtggcgtgtttgaatgtttcgag

SEQ ID NO: 375 - DNA, 194 bp, Homo sapiens
  source 1..194: mol_type = other DNA
agtgaacggttccaagtggacttattttggtcctgatggggagaatagctggtccaagaagtacccgtcgtgtgggggcctgctgcagtcccccatagacctgcacagtgacatcctccagtatgacgccagcctcacgcccctcgagttccaaggctacaatctgtctgccaacaagcagtttctcctgacca

SEQ ID NO: 376 - DNA, 197 bp, Homo sapiens
  source 1..197: mol_type = other DNA
gccttcaagatggagacagagttcttaggatcaatggtgtctttgtggacaaagaagaacatatgcaggttgtggatctggtcagaaagagtgggaattcagtgactttactagttctggatggggattcctatgagaaagcagtgaaaacacgggtggacttgaaagagttgggtcaaagtcagaaggagcaaggt

SEQ ID NO: 377 - DNA, 216 bp, Homo sapiens
  source 1..216: mol_type = other DNA
gaaacaatagccatgtccacgatgcacacagatgtttccaaaacttctttgaagcaagcaagcccagtcgcatttaaaaaaatcaacaacaatgatgataatgaaaaaatctgaaactacttatagcctatggtcccggatgacatctgtttaaaaacaagcacaacctgcaacatactttgattacctgttctcccaaggaatggggttgaaatc

SEQ ID NO: 378 - DNA, 168 bp, Homo sapiens
  source 1..168: mol_type = other DNA
tgatggtctttgtggcactgctcgttttctatatcaccaaaaggaaaaaacagaggagtcggagaaatgatgaggagctggagacaagagcccacagagtagctactgaagaaaggggccggaagccccaccaaattccagcttcaacccctcagaatccagcaactt

SEQ ID NO: 379 - DNA, 199 bp, Homo sapiens
  source 1..199: mol_type = other DNA
ataccccgatgaacaggaaatcaatgcctgaggctgacttcagctcctggacacccttagaattcctagttgaaactttccatgactggatcacagggaaaaaccgaccgagctcaggaagcctaatccaggtggtaaccacagaaggaaggacggaactcaccccagcatatttttaggcctcatctcagtgcctatg

SEQ ID NO: 380 - DNA, 203 bp, Homo sapiens
  source 1..203: mol_type = other DNA
tgcgactctgcggaaatacctgaaatacagcaaaaatatcctggaccggcaagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttc

SEQ ID NO: 381 - DNA, 197 bp, Homo sapiens
  source 1..197: mol_type = other DNA
catcaatgttggccctgtggtggctggagtgattggcgctcgcaggccccagtacgacatctggggaaacacagtcaacgtggccagtcggatggatagcacaggggtccagggcagaatccaggtgactgaggaagtccaccggctgctgagaaggtgcccctaccactttgtgtgccgaggcaaagtcagtgtca

SEQ ID NO: 382 - DNA, 194 bp, Homo sapiens
  source 1..194: mol_type = other DNA
tttgctggacatgagactggaaggctgtctggggctgccgacacacaagctctgttgaggaatgaccaggtctatcagcccctccgagatcgagatgatgctcagtacagccaccttggaggaaactgggctcggaacaagtgaacctgagactggtggcttctagaagcagccattaccaactgtaccttccc

SEQ ID NO: 383 - DNA, 198 bp, Homo sapiens
  source 1..198: mol_type = other DNA
ttcaacaccagatccgagctgttccctttgagaaccttaacatccattgtggggatgccatggacttaggcttagaggccatttttgatcaagttgtgagaagaaatcggggtggatggtgtctccaggtcaatcatcttctgtactgggctctgaccactattggttttgagaccacgatgttgggagggtatgttt

SEQ ID NO: 384 - DNA, 197 bp, Homo sapiens
  source 1..197: mol_type = other DNA
ttattcctcgtggtcatcgaagaggtcgaattgatgacttgcgataccagatagatgataaaccaaacaaccagattcgaatatccaagcaactcgcagagtttgtgccattggattattctgttcctatagaaatccccactataaaatgtaaaccagacaaacttccattattcaaacggcagtatgaaaaccac

SEQ ID NO: 385 - DNA, 189 bp, Homo sapiens
  source 1..189: mol_type = other DNA
catcctgaaaaccccaaagctgtggagacttttcagaaactgcagaaggcaaaggagattctgaccaatgaagagagtcgagcccgctatgaccactggcgaaggagccagatgtcgatgccattccagcagtgggaagctttgaatgactcagtgaagacgtcaatgcactgggttgtcagaggtaaa

SEQ ID NO: 386 - DNA, 187 bp, Homo sapiens
  source 1..187: mol_type = other DNA
gacatagttcgagatggcaggctctatgcatctgagaaccatcaggaaatacttaaggataagaaacttatcaaggctctgtttgatgtcctggcccatccccagaactatttcaagtacacagcccaggagtcccgagagatgtttccaagatcgttcatccgattgctacgttccaaagtgtcca

SEQ ID NO: 387 - DNA, 194 bp, Homo sapiens
  source 1..194: mol_type = other DNA
aaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggt

SEQ ID NO: 388 - DNA, 152 bp, Homo sapiens
  source 1..152: mol_type = other DNA
cagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattg

SEQ ID NO: 389 - DNA, 199 bp, Homo sapiens
  source 1..199: mol_type = other DNA
catcttcatgtggtaccggagcaaggaaagagggcagaggggcttctcccaggaggagctggagacgtgcatgattaaccaggcccctggctgccccgactacagcatcctctccttcatgggcgcgttccatgggaggaccatgggttgcttagcgaccacgcactctaaagccattcacaagatcgacatcccttcc

SEQ ID NO: 390 - DNA, 163 bp, Homo sapiens
  source 1..163: mol_type = other DNA
gatggcatgacgttctttggatcagaagagcttccccagaatctaacctgcactcccgatggtggttcaggagactcttcctgatctttctagaaggggtaaagtggggttgaacaaggccaagccgttagctctgctgctgctcagtttcccagcctagttt

SEQ ID NO: 391 - DNA, 154 bp, Homo sapiens
  source 1..154: mol_type = other DNA
ttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcg

SEQ ID NO: 392 - DNA, 191 bp, Homo sapiens
  source 1..191: mol_type = other DNA
tgccctgtcttactgtcattcgaagagagttattcatagagacattaagccagagaacttacttcttggatcagctggagagcttaaaattgcagattttgggtggtcagtacatgctccatcttccaggaggaccactctctgtggcaccctggactacctgccccctgaaatgattgaaggtcggatgc

SEQ ID NO: 393 - DNA, 191 bp, Homo sapiens
  source 1..191: mol_type = other DNA
tggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacag

SEQ ID NO: 394 - DNA, 200 bp, Homo sapiens
  source 1..200: mol_type = other DNA
cgtcagttggtgagtgtccggccccagtaccagtgaaggacaagaaacttctggaggtcaaactgggggagctgccaagctggatcttgatgcgggacttcagtcctagtggcattttcggagcgtttcaaagaggttactaccggtactacaacaagtacatcaatgtgaagaaggggagcatctcggggattaccatg

SEQ ID NO: 395 - DNA, 172 bp, Homo sapiens
  source 1..172: mol_type = other DNA
tcccactgcttccatttctgcaaaagtcaacaactctagcttaattggagtaggctatactcagactctgaggcctggtgtgaagcttacactctctgctctggtagatgggaagagcattaatgctggaggccacaaggttgggctcgccctggagttggaggcttaatcc

SEQ ID NO: 396 - DNA, 191 bp, Homo sapiens
  source 1..191: mol_type = other DNA
gctcaaagaatacatgatcctcaactgctaacagagagagctttacatcatggaattgatttggagaaaattaaggcttacattgattccttccgctttggagcccctcctcatgctggtggaggcattggattggaacgagttactatgctgtttctgggattgcataatgttcgtcagacctccatgtt

SEQ ID NO: 397 - DNA, 157 bp, Homo sapiens
  source 1..157: mol_type = other DNA
tggatcacctcacagtttcaggagatgtgacatttggaaaaaatgtttcattaaagggaacggttatcatcattgcaaatcatggtgacagaattgatatcccacctggagcagtattagagaacaagattgtgtctggaaaccttcgcatcttgga

SEQ ID NO: 398 - DNA, 186 bp, Homo sapiens
  source 1..186: mol_type = other DNA
aattcagcatcccagactctctgctgatccgaagaatcacaggaaggctgattcaccccaagagtggccgttcctaccacgaggagttcaaccctccaaaagagcccatgaaagatgacatcaccggggaacccttgatccgtcgatcagatgataatgaaaaggccttgaaaatccgcctgcaag

SEQ ID NO: 399 - DNA, 200 bp, Homo sapiens
  source 1..200: mol_type = other DNA
ctcagtgctcatctctatccagtccctgatgactgagaacccctatcacaatgagcccggctttgaacaggagagacatccaggagacagcaaaaactataatgaatgtatccggcacgagaccatcagagttgcagtctgtgacatgatggaaggaaagtgtccctgtcctgaacccctacgaggggtgatggagaagt

SEQ ID NO: 400 - DNA, 153 bp, Homo sapiens
  source 1..153: mol_type = other DNA
gcctccccatctttactgagtaataggccacctcccccagcccgagaccctcccagtcggggagcagctcctccacccccaccacctgtgatccgaaatggtgccagggatgctccccctcccccaccaccataccgaatgcatgggtcagaa

SEQ ID NO: 401 - DNA, 165 bp, Homo sapiens
  source 1..165: mol_type = other DNA
cctttcagtgggacatctggcttctttatataattatgacatgaatcagtatgaaaatgctgagaaactttatttgcgatctatagcaattgggaagaaactttttggtgagggctacagtggactagaatatgattatcgaggtctcattaaactttacaactc

SEQ ID NO: 402 - DNA, 160 bp, Homo sapiens
  source 1..160: mol_type = other DNA
gtcaactatggcctcaaacacaaagtcctccagtcgcagctggatactctgctccaggggcaggagagcattaagagctgcagcaacttcacagcgcaggccctcaaccatggcacggagaccgaggtcctactggtgaagaagcagatgagcgagaagc

SEQ ID NO: 403 - DNA, 192 bp, Homo sapiens
  source 1..192: mol_type = other DNA
tgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggt

SEQ ID NO: 404 - DNA, 100 bp, synthetic construct
  source 1..100: mol_type = other DNA
catcatgtttcccagggcaacttgaggtctgacttttggctccctcgccctaagaggctcttcctctcttttgctcatccaccttcacaccacttgtgag

SEQ ID NO: 405 - DNA, 100 bp, synthetic construct
  source 1..100: mol_type = other DNA
ccagggaaggttcatatcacaaagacgaccctagcgtgcttgaatggggactacgaggtagaaccgggttacggacatgagaggaacagtttcttgaaaa

SEQ ID NO: 406 - DNA, 100 bp, synthetic construct
  source 1..100: mol_type = other DNA
caagagtggccgttcctaccacgaggagttcaaccctccaaaagagcccatgaaagatgacatcaccggggaacccttgatccgtcgatcagatgataat

SEQ ID NO: 407 - DNA, 100 bp, synthetic construct
  source 1..100: mol_type = other DNA
gtggtgcagtccttcctgatttctcagaatgtcgagggaccgagctgctgagggaggacctcagttaaccaattaccttttcccggattccagggaattc

SEQ ID NO: 408 - DNA, 100 bp, synthetic construct
  source 1..100: mol_type = other DNA
ccctccgtgaggaacacaatctcaatcgttgctgaatcctttcatatcctaataggaattaacctccaaataaaacatgactggtacgtgtgctgattgc

SEQ ID NO: 409 - DNA, 100 bp, synthetic construct
  source 1..100: mol_type = other DNA
agctccagttggaggtccaaaacgtgttctcgtgactcagcaatttccttgtcagaatccattacctgtaaatagtggccaggctcagcgggtcttgtgt

SEQ ID NO: 410 - DNA, 100 bp, synthetic construct
  source 1..100: mol_type = other DNA
gttactctctgacctatatctacactgggctgtccaagcatgttgaagacgtccccgcgtttcaggcccttggctcactcaatgacctccagttctttag

SEQ ID NO: 411 - DNA, 100 bp, synthetic construct
  source 1..100: mol_type = other DNA
ccatattcttccccgaatcacacagacagtttctgacaggcgcaactcctccattttcctcccgcaggtgagaaccctgtggagatgagtcagtgccatg

SEQ ID NO: 412 - DNA, 100 bp, synthetic construct
  source 1..100: mol_type = other DNA
tgggtctcactacaagcagcctatctgcttaagagactctggagtttcttatgtgccctggtggacacttgcccaccatcctgtgagtaaaagtgaaata

SEQ ID NO: 413 - DNA, 100 bp, synthetic construct
  source 1..100: mol_type = other DNA
tatctactggatgagttccgctgggagatggaacatagcacgtttctctctggcctggtactggctacccttctctcgcaagtgagccccttcaagatac

SEQ ID NO: 414 - DNA, 100 bp, synthetic construct
  source 1..100: mol_type = other DNA
tcctcatgctggtggaggcattggattggaacgagttactatgctgtttctgggattgcataatgttcgtcagacctccatgttccctcgtgatcccaaa

SEQ ID NO: 415 - DNA, 100 bp, synthetic construct
  source 1..100: mol_type = other DNA
agaaggcaaaggagattctgaccaatgaagagagtcgagcccgctatgaccactggcgaaggagccagatgtcgatgccattccagcagtgggaagcttt

SEQ ID NO: 416 - DNA, 100 bp, synthetic construct
  source 1..100: mol_type = other DNA
acagacacgtttgagtccatgcccaatcccgagggccggtatacattcggcgccagctgtgtgactgcctgtccctacaactacctttctacggacgtgg

SEQ ID NO: 417 - DNA, 100 bp, synthetic construct
  source 1..100: mol_type = other DNA
aggaaccagggaaaatgtgtagagggcatggtggagatcttcgacatgctgctggctacatcatctcggttccgcatgatgaatctgcagggagaggagt

SEQ ID NO: 418 - DNA, 100 bp, synthetic construct
  source 1..100: mol_type = other DNA
tctccatccccagggggttgggaggatgtctttagattttaggactctgtgataaatattccacagcctggtgtgaggaagcttggaccaaatgcttccc

SEQ ID NO: 419 - DNA, 100 bp, synthetic construct
  source 1..100: mol_type = other DNA
aacttcctggctccttcactgaacatgcctagtccaacattttttcccagtgagtcacatcctgggatccagtgtataaatccaatatcatgtcttgtgc

SEQ ID NO: 420 - DNA, 100 bp, synthetic construct
  source 1..100: mol_type = other DNA
attgggtccctggacaatatcacccacgtccctggcggaggaaataaaaagattgaaacccacaagctgaccttccgcgagaacgccaaagccaagacag

SEQ ID NO: 421 - DNA, 100 bp, synthetic construct
  source 1..100: mol_type = other DNA
taatgctattgcaagcctttttgcttggactggagcacaagctatgtatcaaggattctggagtgaagcagatgttactcgaccttttgtctcccaggct

SEQ ID NO: 422 - DNA, 100 bp, synthetic construct
  source 1..100: mol_type = other DNA
tgaaatcctgtggacactgggcgaattactttttagatctgtagctctgactcctcaggcataaaatgggaataatgcttttacagtttagtggcggaac

SEQ ID NO: 423 - DNA, 100 bp, synthetic construct
  source 1..100: mol_type = other DNA
acatacttctcagctgcaaatattatggagaattggggcacccacaggaatgaagagagaaagcagctccctaacttcaaaaccattttggtacctgaca

SEQ ID NO: 424 - DNA, 100 bp, synthetic construct
  source 1..100: mol_type = other DNA
accgagggccacctggtccgggtggttgagaagtgtagcccagcagagaaggctggccttcaagatggagacagagttcttaggatcaatggtgtctttg

SEQ ID NO: 425 - DNA, 100 bp, synthetic construct
  source 1..100: mol_type = other DNA
agcatcaggctgtcattatggtgtccttacctgtgggagctgtaaggtcttctttaagagggcaatggaagggcagcacaactacttatgtgctggaaga

SEQ ID NO: 426 - DNA, 100 bp, synthetic construct
  source 1..100: mol_type = other DNA
acagtgtaatacatttatgtgaagtaaagacatgcgagactggtggcctgcaaatagcatccgttgatctgtgttaactgcatagggagggctctgcata

SEQ ID NO: 427 - DNA, 100 bp, synthetic construct
  source 1..100: mol_type = other DNA
cgtaaagccatccgcacgctcagaaaggccgtccacagggagcagtttcacctccagctctcaggcatgaacctcgacgtggctaaaaagcctcccagaa

SEQ ID NO: 428 - DNA, 100 bp, synthetic construct
  source 1..100: mol_type = other DNA
gatcgaactgaaggctatttacgagcagactcacaagagccctcccactttgattctcagcagcctgcagtcttggaagaagaagaggtcatgatagctc

SEQ ID NO: 429 - DNA, 100 bp, synthetic construct
  source 1..100: mol_type = other DNA
atttctatgtgtaatttctgagccattgtactgtctgggctgggggggacactgtccaagggagtggcccctatgagtttatattttaaccactgcttca

SEQ ID NO: 430 - DNA, 100 bp, synthetic construct
  source 1..100: mol_type = other DNA
acctttgatgagctccagaagactttaaatgtgcgcaagagtgtgctgcttatggaattggaggtcaactatggcctcaaacacaaagtcctccagtcgc

SEQ ID NO: 431 - DNA, 100 bp, synthetic construct
  source 1..100: mol_type = other DNA
attcatgcattgatcacaggcccatttgacactccttatgaagggggtttcttcctgttcgtgtttcggtgtccgcccgactatcccatccacccacctc

SEQ ID NO: 432 - DNA, 100 bp, synthetic construct
  source 1..100: mol_type = other DNA
tgctggtgatgtcaaacctctatagtcttaatgcaggatctctgacaatgagtgaaaagcgggaatttcctacagtgcccttggttaaattaggcagttc

SEQ ID NO: 433 - DNA, 100 bp, synthetic construct
  source 1..100: mol_type = other DNA
ttcaacgtccggttcatctaatacagacactggtaaagttactgggaccttggagaccaaatacaagtggtgtgagtatggtctgactttcacagaaaag

SEQ ID NO: 434 - DNA, 100 bp, synthetic construct
  source 1..100: mol_type = other DNA
gcaagccctgctagccacatgaggaacaagtttccgtgtcttctgccttcctcttggggaaaggtgccttgttgtgatgaattaactcactgttagggca